SEQUENCE LISTING (17 sequences)

SEQ ID NO: 1 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPTGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 2 - protein, 1366 aa, Homo sapiens
MLSFVDTRTLLLLAVTLCLATCQSLQEETVRKGPAGDRGPRGERGPPGPPGRDGEDGPTGPPGPPGPPGPPGLGGNFAAQYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPAGPAGPRGEVGLPGLSGPVGPPGNPGANGLTGAKGAAGLPGVAGAPGLPGPRGIPGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPPGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAPGAVGAPGPAGATGDRGEAGAAGPAGPAGPRGSPGERGEVGPAGPNGFAGPAGAAGQPGAKGERGAKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGLKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMDAIKVYCDFSTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADQEFFVDIGPVCFK

SEQ ID NO: 3 - protein, 1487 aa, Homo sapiens
MIRLGAPQTLVLLTLLVAAVLRCQGQDVQEAGSCVQDGQRYNDKDVWKPEPCRICVCDTGTVLCDDIICEDVKDCLSPEIPFGECCPICPTDLATASGQPGPKGQKGEPGDIKDIVGPKGPPGPQGPAGEQGPRGDRGDKGEKGAPGPRGRDGEPGTPGNPGPPGPPGPPGPPGLGGNFAAQMAGGFDEKAGGAQLGVMQGPMGPMGPRGPPGPAGAPGPQGFQGNPGEPGEPGVSGPMGPRGPPGPPGKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPGPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRADQAAGGLRQHDAEVDATLKSLNNQIESIRSPEGSRKNPARTCRDLKLCHPEWKSGDYWIDPNQGCTLDAMKVFCNMETGETCVYPNPANVPKKNWWSSKSKEKKHIWFGETINGGFHFSYGDDNLAPNTANVQMTFLRLLSTEGSQNITYHCKNSIAYLDEAAGNLKKALLIQGSNDVEIRAEGNSRFTYTALKDGCTKHTGKWGKTVIEYRSQKTSRLPIIDIAPMDIGGPEQEFGVDIGPVCFL

SEQ ID NO: 4 - protein, 1466 aa, Homo sapiens
MMSFVQKGSWLLLALLHPTIILAQQEAVEGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGIPGQPGSPGSPGPPGICESCPTGPQNYSPQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYGDEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVHLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 5 - protein, 1669 aa, Homo sapiens
MGPRLSVWLLLLPAALLLHEEHSRAAAKGGCAGSGCGKCDCHGVKGQKGERGLPGLQGVIGFPGMQGPEGPQGPPGQKGDTGEPGLPGTKGTRGPPGASGYPGNPGLPGIPGQDGPPGPPGIPGCNGTKGERGPLGPPGLPGFAGNPGPPGLPGMKGDPGEILGHVPGMLLKGERGFPGIPGTPGPPGLPGLQGPVGPPGFTGPPGPPGPPGPPGEKGQMGLSFQGPKGDKGDQGVSGPPGVPGQAQVQEKGDFATKGEKGQKGEPGFQGMPGVGEKGEPGKPGPRGKPGKDGDKGEKGSPGFPGEPGYPGLIGRQGPQGEKGEAGPPGPPGIVIGTGPLGEKGERGYPGTPGPRGEPGPKGFPGLPGQPGPPGLPVPGQAGAPGFPGERGEKGDRGFPGTSLPGPSGRDGLPGPPGSPGPPGQPGYTNGIVECQPGPPGDQGPPGIPGQPGFIGEIGEKGQKGESCLICDIDGYRGPPGPQGPPGEIGFPGQPGAKGDRGLPGRDGVAGVPGPQGTPGLIGQPGAKGEPGEFYFDLRLKGDKGDPGFPGQPGMTGRAGSPGRDGHPGLPGPKGSPGSVGLKGERGPPGGVGFPGSRGDTGPPGPPGYGPAGPIGDKGQAGFPGGPGSPGLPGPKGEPGKIVPLPGPPGAEGLPGSPGFPGPQGDRGFPGTPGRPGLPGEKGAVGQPGIGFPGPPGPKGVDGLPGDMGPPGTPGRPGFNGLPGNPGVQGQKGEPGVGLPGLKGLPGLPGIPGTPGEKGSIGVPGVPGEHGAIGPPGLQGIRGEPGPPGLPGSVGSPGVPGIGPPGARGPPGGQGPPGLSGPPGIKGEKGFPGFPGLDMPGPKGDKGAQGLPGITGQSGLPGLPGQQGAPGIPGFPGSKGEMGVMGTPGQPGSPGPVGAPGLPGEKGDHGFPGSSGPRGDPGLKGDKGDVGLPGKPGSMDKVDMGSMKGQKGDQGEKGQIGPIGEKGSRGDPGTPGVPGKDGQAGQPGQPGPKGDPGISGTPGAPGLPGPKGSVGGMGLPGTPGEKGVPGIPGPQGSPGLPGDKGAKGEKGQAGPPGIGIPGLRGEKGDQGIAGFPGSPGEKGEKGSIGIPGMPGSPGLKGSPGSVGYPGSPGLPGEKGDKGLPGLDGIPGVKGEAGLPGTPGPTGPAGQKGEPGSDGIPGSAGEKGEPGLPGRGFPGFPGAKGDKGSKGEVGFPGLAGSPGIPGSKGEQGFMGPPGPQGQPGLPGSPGHATEGPKGDRGPQGQPGLPGLPGPMGPPGLPGIDGVKGDKGNPGWPGAPGVPGPKGDPGFQGMPGIGGSPGITGSKGDMGPPGVPGFQGPKGLPGLQGIKGDQGDQGVPGAKGLPGPPGPPGPYDIIKGEPGLPGPEGPPGLKGLQGLPGPKGQQGVTGLVGIPGPPGIPGFDGAPGQKGEMGPAGPTGPRGFPGPPGPDGLPGSMGPPGTPSVDHGFLVTRHSQTIDDPQCPSGTKILYHGYSLLYVQGNERAHGQDLGTAGSCLRKFSTMPFLFCNINNVCNFASRNDYSYWLSTPEPMPMSMAPITGENIRPFISRCAVCEAPAMVMAVHSQTIQIPPCPSGWSSLWIGYSFVMHTSAGAEGSGQALASPGSCLEEFRSAPFIECHGRGTCNYYANAYSFWLATIERSEMFKKPTPSTLKAGELRTHVSRCQVCMRRT

SEQ ID NO: 6 - protein, 1712 aa, Homo sapiens
MGRDQRAVAGPALRRWLLLGTVTVGFLAQSVLAGVKKFDVPCGGRDCSGGCQCYPEKGGRGQPGPVGPQGYNGPPGLQGFPGLQGRKGDKGERGAPGVTGPKGDVGARGVSGFPGADGIPGHPGQGGPRGRPGYDGCNGTQGDSGPQGPPGSEGFTGPPGPQGPKGQKGEPYALPKEERDRYRGEPGEPGLVGFQGPPGRPGHVGQMGPVGAPGRPGPPGPPGPKGQQGNRGLGFYGVKGEKGDVGQPGPNGIPSDTLHPIIAPTGVTFHPDQYKGEKGSEGEPGIRGISLKGEEGIMGFPGLRGYPGLSGEKGSPGQKGSRGLDGYQGPDGPRGPKGEAGDPGPPGLPAYSPHPSLAKGARGDPGFPGAQGEPGSQGEPGDPGLPGPPGLSIGDGDQRRGLPGEMGPKGFIGDPGIPALYGGPPGPDGKRGPPGPPGLPGPPGPDGFLFGLKGAKGRAGFPGLPGSPGARGPKGWKGDAGECRCTEGDEAIKGLPGLPGPKGFAGINGEPGRKGDRGDPGQHGLPGFPGLKGVPGNIGAPGPKGAKGDSRTITTKGERGQPGVPGVPGMKGDDGSPGRDGLDGFPGLPGPPGDGIKGPPGDPGYPGIPGTKGTPGEMGPPGLGLPGLKGQRGFPGDAGLPGPPGFLGPPGPAGTPGQIDCDTDVKRAVGGDRQEAIQPGCIGGPKGLPGLPGPPGPTGAKGLRGIPGFAGADGGPGPRGLPGDAGREGFPGPPGFIGPRGSKGAVGLPGPDGSPGPIGLPGPDGPPGERGLPGEVLGAQPGPRGDAGVPGQPGLKGLPGDRGPPGFRGSQGMPGMPGLKGQPGLPGPSGQPGLYGPPGLHGFPGAPGQEGPLGLPGIPGREGLPGDRGDPGDTGAPGPVGMKGLSGDRGDAGFTGEQGHPGSPGFKGIDGMPGTPGLKGDRGSPGMDGFQGMPGLKGRPGFPGSKGEAGFFGIPGLKGLAGEPGFKGSRGDPGPPGPPPVILPGMKDIKGEKGDEGPMGLKGYLGAKGIQGMPGIPGLSGIPGLPGRPGHIKGVKGDIGVPGIPGLPGFPGVAGPPGITGFPGFIGSRGDKGAPGRAGLYGEIGATGDFGDIGDTINLPGRPGLKGERGTTGIPGLKGFFGEKGTEGDIGFPGITGVTGVQGPPGLKGQTGFPGLTGPPGSQGELGRIGLPGGKGDDGWPGAPGLPGFPGLRGIRGLHGLPGTKGFPGSPGSDIHGDPGFPGPPGERGDPGEANTLPGPVGVPGQKGDQGAPGERGPPGSPGLQGFPGITPPSNISGAPGDKGAPGIFGLKGYRGPPGPPGSAALPGSKGDTGNPGAPGTPGTKGWAGDSGPQGRPGVFGLPGEKGPRGEQGFMGNTGPTGAVGDRGPKGPKGDPGFPGAPGTVGAPGIAGIPQKIAVQPGTVGPQGRRGPPGAPGEMGPQGPPGEPGFRGAPGKAGPQGRGGVSAVPGFRGDEGPIGHQGPIGQEGAPGRPGSPGLPGMPGRSVSIGYLLVKHSQTDQEPMCPVGMNKLWSGYSLLYFEGQEKAHNQDLGLAGSCLARFSTMPFLYCNPGDVCYYASRNDKSYWLSTTAPLPMMPVAEDEIKPYISRCSVCEAPAIAIAVHSQDVSIPHCPAGWRSLWIGYSFLMHTAAGDEGGGQSLVSPGSCLEDFRATPFIECNGGRGTCHYYANKYSFWLTTIPEQSFQGSPSADTLKAGLIRTHISRCQVCMKNL

SEQ ID NO: 7 - protein, 1670 aa, Homo sapiens
MSARTAPRPQVLLLPLLLVLLAAAPAASKGCVCKDKGQCFCDGAKGEKGEKGFPGPPGSPGQKGFTGPEGLPGPQGPKGFPGLPGLTGSKGVRGISGLPGFSGSPGLPGTPGNTGPYGLVGVPGCSGSKGEQGFPGLPGTLGYPGIPGAAGLKGQKGAPAKEEDIELDAKGDPGLPGAPGPQGLPGPPGFPGPVGPPGPPGFFGFPGAMGPRGPKGHMGERVIGHKGERGVKGLTGPPGPPGTVIVTLTGPDNRTDLKGEKGDKGAMGEPGPPGPSGLPGESYGSEKGAPGDPGLQGKPGKDGVPGFPGSEGVKGNRGFPGLMGEDGIKGQKGDIGPPGFRGPTEYYDTYQEKGDEGTPGPPGPRGARGPQGPSGPPGVPGSPGSSRPGLRGAPGWPGLKGSKGERGRPGKDAMGTPGSPGCAGSPGLPGSPGPPGPPGDIVFRKGPPGDHGLPGYLGSPGIPGVDGPKGEPGLLCTQCPYIPGPPGLPGLPGLHGVKGIPGRQGAAGLKGSPGSPGNTGLPGFPGFPGAQGDPGLKGEKGETLQPEGQVGVPGDPGLRGQPGRKGLDGIPGTPGVKGLPGPKGELALSGEKGDQGPPGDPGSPGSPGPAGPAGPPGYGPQGEPGLQGTQGVPGAPGPPGEAGPRGELSVSTPVPGPPGPPGPPGHPGPQGPPGIPGSLGKCGDPGLPGPDGEPGIPGIGFPGPPGPKGDQGFPGTKGSLGCPGKMGEPGLPGKPGLPGAKGEPAVAMPGGPGTPGFPGERGNSGEHGEIGLPGLPGLPGTPGNEGLDGPRGDPGQPGPPGEQGPPGRCIEGPRGAQGLPGLNGLKGQQGRRGKTGPKGDPGIPGLDRSGFPGETGSPGIPGHQGEMGPLGQRGYPGNPGILGPPGEDGVIGMMGFPGAIGPPGPPGNPGTPGQRGSPGIPGVKGQRGTPGAKGEQGDKGNPGPSEISHVIGDKGEPGLKGFAGNPGEKGNRGVPGMPGLKGLKGLPGPAGPPGPRGDLGSTGNPGEPGLRGIPGSMGNMGMPGSKGKRGTLGFPGRAGRPGLPGIHGLQGDKGEPGYSEGTRPGPPGPTGDPGLPGDMGKKGEMGQPGPPGHLGPAGPEGAPGSPGSPGLPGKPGPHGDLGFKGIKGLLGPPGIRGPPGLPGFPGSPGPMGIRGDQGRDGIPGPAGEKGETGLLRAPPGPRGNPGAQGAKGDRGAPGFPGLPGRKGAMGDAGPRGPTGIEGFPGPPGLPGAIIPGQTGNRGPPGSRGSPGAPGPPGPPGSHVIGIKGDKGSMGHPGPKGPPGTAGDMGPPGRLGAPGTPGLPGPRGDPGFQGFPGVKGEKGNPGFLGSIGPPGPIGPKGPPGVRGDPGTLKIISLPGSPGPPGTPGEPGMQGEPGPPGPPGNLGPCGPRGKPGKDGKPGTPGPAGEKGNKGSKGEPGPAGSDGLPGLKGKRGDSGSPATWTTRGFVFTRHSQTTAIPSCPEGTVPLYSGFSFLFVQGNQRAHGQDLGTLGSCLQRFTTMPFLFCNVNDVCNFASRNDYSYWLSTPALMPMNMAPITGRALEPYISRCTVCEGPAIAIAVHSQTTDIPPCPHGWISLWKGFSFIMFTSAGSEGTGQALASPGSCLEEFRASPFLECHGRGTCNYYSNSYSFWLASLNPERMFRKPIPSTVKAGELEKIISRCQVCMKKRH

SEQ ID NO: 8 - protein, 1690 aa, Homo sapiens
MWSLHIVLMRCSFRLTKSLATGPWSLILILFSVQYVYGSGKKYIGPCGGRDCSVCHCVPEKGSRGPPGPPGPQGPIGPLGAPGPIGLSGEKGMRGDRGPPGAAGDKGDKGPTGVPGFPGLDGIPGHPGPPGPRGKPGMSGHNGSRGDPGFPGGRGALGPGGPLGHPGEKGEKGNSVFILGAVKGIQGDRGDPGLPGLPGSWGAGGPAGPTGYPGEPGLVGPPGQPGRPGLKGNPGVGVKGQMGDPGEVGQQGSPGPTLLVEPPDFCLYKGEKGIKGIPGMVGLPGPPGRKGESGIGAKGEKGIPGFPGPRGDPGSYGSPGFPGLKGELGLVGDPGLFGLIGPKGDPGNRGHPGPPGVLVTPPLPLKGPPGDPGFPGRYGETGDVGPPGPPGLLGRPGEACAGMIGPPGPQGFPGLPGLPGEAGIPGRPDSAPGKPGKPGSPGLPGAPGLQGLPGSSVIYCSVGNPGPQGIKGKVGPPGGRGPKGEKGNEGLCACEPGPMGPPGPPGLPGRQGSKGDLGLPGWLGTKGDPGPPGAEGPPGLPGKHGASGPPGNKGAKGDMVVSRVKGHKGERGPDGPPGFPGQPGSHGRDGHAGEKGDPGPPGDHEDATPGGKGFPGPLGPPGKAGPVGPPGLGFPGPPGERGHPGVPGHPGVRGPDGLKGQKGDTISCNVTYPGRHGPPGFDGPPGPKGFPGPQGAPGLSGSDGHKGRPGTPGTAEIPGPPGFRGDMGDPGFGGEKGSSPVGPPGPPGSPGVNGQKGIPGDPAFGHLGPPGKRGLSGVPGIKGPRGDPGCPGAEGPAGIPGFLGLKGPKGREGHAGFPGVPGPPGHSCERGAPGIPGQPGLPGYPGSPGAPGGKGQPGDVGPPGPAGMKGLPGLPGRPGAHGPPGLPGIPGPFGDDGLPGPPGPKGPRGLPGFPGFPGERGKPGAEGCPGAKGEPGEKGMSGLPGDRGLRGAKGAIGPPGDEGEMAIISQKGTPGEPGPPGDDGFPGERGDKGTPGMQGRRGEPGRYGPPGFHRGEPGEKGQPGPPGPPGPPGSTGLRGFIGFPGLPGDQGEPGSPGPPGFSGIDGARGPKGNKGDPASHFGPPGPKGEPGSPGCPGHFGASGEQGLPGIQGPRGSPGRPGPPGSSGPPGCPGDHGMPGLRGQPGEMGDPGPRGLQGDPGIPGPPGIKGPSGSPGLNGLHGLKGQKGTKGASGLHDVGPPGPVGIPGLKGERGDPGSPGISPPGPRGKKGPPGPPGSSGPPGPAGATGRAPKDIPDPGPPGDQGPPGPDGPRGAPGPPGLPGSVDLLRGEPGDCGLPGPPGPPGPPGPPGYKGFPGCDGKDGQKGPVGFPGPQGPHGFPGPPGEKGLPGPPGRKGPTGLPGPRGEPGPPADVDDCPRIPGLPGAPGMRGPEGAMGLPGMRGPSGPGCKGEPGLDGRRGVDGVPGSPGPPGRKGDTGEDGYPGGPGPPGPIGDPGPKGFGPGYLGGFLLVLHSQTDQEPTCPLGMPRLWTGYSLLYLEGQEKAHNQDLGLAGSCLPVFSTLPFAYCNIHQVCHYAQRNDRSYWLASAAPLPMMPLSEEAIRPYVSRCAVCEAPAQAVAVHSQDQSIPPCPQTWRSLWIGYSFLMHTGAGDQGGGQALMSPGSCLEDFRAAPFLECQGRQGTCHFFANKYSFWLTTVKADLQFSSAPAPDTLKESQAQRQKISRCQVCVKYS

SEQ ID NO: 9 - protein, 1685 aa, Homo sapiens
MKLRGVSLAAGLFLLALSLWGQPAEAAACYGCSPGSKCDCSGIKGEKGERGFPGLEGHPGLPGFPGPEGPPGPRGQKGDDGIPGPPGPKGIRGPPGLPGFPGTPGLPGMPGHDGAPGPQGIPGCNGTKGERGFPGSPGFPGLQGPPGPPGIPGMKGEPGSIIMSSLPGPKGNPGYPGPPGIQGLPGPTGIPGPIGPPGPPGLMGPPGPPGLPGPKGNMGLNFQGPKGEKGEQGLQGPPGPPGQISEQKRPIDVEFQKGDQGLPGDRGPPGPPGIRGPPGPPGGEKGEKGEQGEPGKRGKPGKDGENGQPGIPGLPGDPGYPGEPGRDGEKGQKGDTGPPGPPGLVIPRPGTGITIGEKGNIGLPGLPGEKGERGFPGIQGPPGLPGPPGAAVMGPPGPPGFPGERGQKGDEGPPGISIPGPPGLDGQPGAPGLPGPPGPAGPHIPPSDEICEPGPPGPPGSPGDKGLQGEQGVKGDKGDTCFNCIGTGISGPPGQPGLPGLPGPPGSLGFPGQKGEKGQAGATGPKGLPGIPGAPGAPGFPGSKGEPGDILTFPGMKGDKGELGSPGAPGLPGLPGTPGQDGLPGLPGPKGEPGGITFKGERGPPGNPGLPGLPGNIGPMGPPGFGPPGPVGEKGIQGVAGNPGQPGIPGPKGDPGQTITQPGKPGLPGNPGRDGDVGLPGDPGLPGQPGLPGIPGSKGEPGIPGIGLPGPPGPKGFPGIPGPPGAPGTPGRIGLEGPPGPPGFPGPKGEPGFALPGPPGPPGLPGFKGALGPKGDRGFPGPPGPPGRTGLDGLPGPKGDVGPNGQPGPMGPPGLPGIGVQGPPGPPGIPGPIGQPGLHGIPGEKGDPGPPGLDVPGPPGERGSPGIPGAPGPIGPPGSPGLPGKAGASGFPGTKGEMGMMGPPGPPGPLGIPGRSGVPGLKGDDGLQGQPGLPGPTGEKGSKGEPGLPGPPGPMDPNLLGSKGEKGEPGLPGIPGVSGPKGYQGLPGDPGQPGLSGQPGLPGPPGPKGNPGLPGQPGLIGPPGLKGTIGDMGFPGPQGVEGPPGPSGVPGQPGSPGLPGQKGDKGDPGISSIGLPGLPGPKGEPGLPGYPGNPGIKGSVGDPGLPGLPGTPGAKGQPGLPGFPGTPGPPGPKGISGPPGNPGLPGEPGPVGGGGHPGQPGPPGEKGKPGQDGIPGPAGQKGEPGQPGFGNPGPPGLPGLSGQKGDGGLPGIPGNPGLPGPKGEPGFHGFPGVQGPPGPPGSPGPALEGPKGNPGPQGPPGRPGLPGPEGPPGLPGNGGIKGEKGNPGQPGLPGLPGLKGDQGPPGLQGNPGRPGLNGMKGDPGLPGVPGFPGMKGPSGVPGSAGPEGEPGLIGPPGPPGLPGPSGQSIIIKGDAGPPGIPGQPGLKGLPGPQGPQGLPGPTGPPGDPGRNGLPGFDGAGGRKGDPGLPGQPGTRGLDGPPGPDGLQGPPGPPGTSSVAHGFLITRHSQTTDAPQCPQGTLQVYEGFSLLYVQGNKRAHGQDLGTAGSCLRRFSTMPFMFCNINNVCNFASRNDYSYWLSTPEPMPMSMQPLKGQSIQPFISRCAVCEAPAVVIAVHSQTIQIPHCPQGWDSLWIGYSFMMHTSAGAEGSGQALASPGSCLEEFRSAPFIECHGRGTCNYYANSYSFWLATVDVSDMFSKPQSETLKAGDLRTRISRCQVCMKRT

SEQ ID NO: 10 - protein, 1691 aa, Homo sapiens
MLINKLWLLLVTLCLTEELAAAGEKSYGKPCGGQDCSGSCQCFPEKGARGRPGPIGIQGPTGPQGFTGSTGLSGLKGERGFPGLLGPYGPKGDKGPMGVPGFLGINGIPGHPGQPGPRGPPGLDGCNGTQGAVGFPGPDGYPGLLGPPGLPGQKGSKGDPVLAPGSFKGMKGDPGLPGLDGITGPQGAPGFPGAVGPAGPPGLQGPPGPPGPLGPDGNMGLGFQGEKGVKGDVGLPGPAGPPPSTGELEFMGFPKGKKGSKGEPGPKGFPGISGPPGFPGLGTTGEKGEKGEKGIPGLPGPRGPMGSEGVQGPPGQQGKKGTLGFPGLNGFQGIEGQKGDIGLPGPDVFIDIDGAVISGNPGDPGVPGLPGLKGDEGIQGLRGPSGVPGLPALSGVPGALGPQGFPGLKGDQGNPGRTTIGAAGLPGRDGLPGPPGPPGPPSPEFETETLHNKESGFPGLRGEQGPKGNLGLKGIKGDSGFCACDGGVPNTGPPGEPGPPGPWGLIGLPGLKGARGDRGSGGAQGPAGAPGLVGPLGPSGPKGKKGEPILSTIQGMPGDRGDSGSQGFRGVIGEPGKDGVPGLPGLPGLPGDGGQGFPGEKGLPGLPGEKGHPGPPGLPGNGLPGLPGPRGLPGDKGKDGLPGQQGLPGSKGITLPCIIPGSYGPSGFPGTPGFPGPKGSRGLPGTPGQPGSSGSKGEPGSPGLVHLPELPGFPGPRGEKGLPGFPGLPGKDGLPGMIGSPGLPGSKGATGDIFGAENGAPGEQGLQGLTGHKGFLGDSGLPGLKGVHGKPGLLGPKGERGSPGTPGQVGQPGTPGSSGPYGIKGKSGLPGAPGFPGISGHPGKKGTRGKKGPPGSIVKKGLPGLKGLPGNPGLVGLKGSPGSPGVAGLPALSGPKGEKGSVGFVGFPGIPGLPGIPGTRGLKGIPGSTGKMGPSGRAGTPGEKGDRGNPGPVGIPSPRRPMSNLWLKGDKGSQGSAGSNGFPGPRGDKGEAGRPGPPGLPGAPGLPGIIKGVSGKPGPPGFMGIRGLPGLKGSSGITGFPGMPGESGSQGIRGSPGLPGASGLPGLKGDNGQTVEISGSPGPKGQPGESGFKGTKGRDGLIGNIGFPGNKGEDGKVGVSGDVGLPGAPGFPGVAGMRGEPGLPGSSGHQGAIGPLGSPGLIGPKGFPGFPGLHGLNGLPGTKGTHGTPGPSITGVPGPAGLPGPKGEKGYPGIGIGAPGKPGLRGQKGDRGFPGLQGPAGLPGAPGISLPSLIAGQPGDPGRPGLDGERGRPGPAGPPGPPGPSSNQGDTGDPGFPGIPGPKGPKGDQGIPGFSGLPGELGLKGMRGEPGFMGTPGKVGPPGDPGFPGMKGKAGPRGSSGLQGDPGQTPTAEAVQVPPGPLGLPGIDGIPGLTGDPGAQGPVGLQGSKGLPGIPGKDGPSGLPGPPGALGDPGLPGLQGPPGFEGAPGQQGPFGMPGMPGQSMRVGYTLVKHSQSEQVPPCPIGMSQLWVGYSLLFVEGQEKAHNQDLGFAGSCLPRFSTMPFIYCNINEVCHYARRNDKSYWLSTTAPIPMMPVSQTQIPQYISRCSVCEAPSQAIAVHSQDITIPQCPLGWRSLWIGYSFLMHTAAGAEGGGQSLVSPGSCLEDFRATPFIECSGARGTCHYFANKYSFWLTTVEERQQFGELPVSETLKAGQLHTRVSRCQVCMKSL

SEQ ID NO: 11 - protein, 1838 aa, Homo sapiens
MDVHTRWKARSALRPGAPLLPPLLLLLLWAPPPSRAAQPADLLKVLDFHNLPDGITKTTGFCATRRSSKGPDVAYRVTKDAQLSAPTKQLYPASAFPEDFSILTTVKAKKGSQAFLVSIYNEQGIQQIGLELGRSPVFLYEDHTGKPGPEDYPLFRGINLSDGKWHRIALSVHKKNVTLILDCKKKTTKFLDRSDHPMIDINGIIVFGTRILDEEVFEGDIQQLLFVSDHRAAYDYCEHYSPDCDTAVPDTPQSQDPNPDEYYTEGDGEGETYYYEYPYYEDPEDLGKEPTPSKKPVEAAKETTEVPEELTPTPTEAAPMPETSEGAGKEEDVGIGDYDYVPSEDYYTPSPYDDLTYGEGEENPDQPTDPGAGAEIPTSTADTSNSSNPAPPPGEGADDLEGEFTEETIRNLDENYYDPYYDPTSSPSEIGPGMPANQDTIYEGIGGPRGEKGQKGEPAIIEPGMLIEGPPGPEGPAGLPGPPGTMGPTGQVGDPGERGPPGRPGLPGADGLPGPPGTMLMLPFRFGGGGDAGSKGPMVSAQESQAQAILQQARLALRGPAGPMGLTGRPGPVGPPGSGGLKGEPGDVGPQGPRGVQGPPGPAGKPGRRGRAGSDGARGMPGQTGPKGDRGFDGLAGLPGEKGHRGDPGPSGPPGPPGDDGERGDDGEVGPRGLPGEPGPRGLLGPKGPPGPPGPPGVTGMDGQPGPKGNVGPQGEPGPPGQQGNPGAQGLPGPQGAIGPPGEKGPLGKPGLPGMPGADGPPGHPGKEGPPGEKGGQGPPGPQGPIGYPGPRGVKGADGIRGLKGTKGEKGEDGFPGFKGDMGIKGDRGEIGPPGPRGEDGPEGPKGRGGPNGDPGPLGPPGEKGKLGVPGLPGYPGRQGPKGSIGFPGFPGANGEKGGRGTPGKPGPRGQRGPTGPRGERGPRGITGKPGPKGNSGGDGPAGPPGERGPNGPQGPTGFPGPKGPPGPPGKDGLPGHPGQRGETGFQGKTGPPGPPGVVGPQGPTGETGPMGERGHPGPPGPPGEQGLPGLAGKEGTKGDPGPAGLPGKDGPPGLRGFPGDRGLPGPVGALGLKGNEGPPGPPGPAGSPGERGPAGAAGPIGIPGRPGPQGPPGPAGEKGAPGEKGPQGPAGRDGLQGPVGLPGPAGPVGPPGEDGDKGEIGEPGQKGSKGDKGEQGPPGPTGPQGPIGQPGPSGADGEPGPRGQQGLFGQKGDEGPRGFPGPPGPVGLQGLPGPPGEKGETGDVGQMGPPGPPGPRGPSGAPGADGPQGPPGGIGNPGAVGEKGEPGEAGEPGLPGEGGPPGPKGERGEKGESGPSGAAGPPGPKGPPGDDGPKGSPGPVGFPGDPGPPGEPGPAGQDGPPGDKGDDGEPGQTGSPGPTGEPGPSGPPGKRGPPGPAGPEGRQGEKGAKGEAGLEGPPGKTGPIGPQGAPGKPGPDGLRGIPGPVGEQGLPGSPGPDGPPGPMGPPGLPGLKGDSGPKGEKGHPGLIGLIGPPGEQGEKGDRGLPGPQGSSGPKGEQGITGPSGPIGPPGPPGLPGPPGPKGAKGSSGPTGPKGEAGHPGPPGPPGPPGEVIQPLPIQASRTRRNIDASQLLDDGNGENYVDYADGMEEIFGSLNSLKLEIEQMKRPLGTQQNPARTCKDLQLCHPDFPDGEYWVDPNQGCSRDSFKVYCNFTAGGSTCVFPDKKSEGARITSWPKENPGSWFSEFKRGKLLSYVDAEGNPVGVVQMTFLRLLSASAHQNVTYHCYQSVAWQDAATGSYDKALRFLGSNDEEMSYDNNPYIRALVDGCATKKGYQKTVLEIDTPKVEQVPIVDIMFNDFGEASQKFGFEVGPACFMG

SEQ ID NO: 12 - protein, 1499 aa, Homo sapiens
MMANWAEARPLLILIVLLGQFVSIKAQEEDEDEGYGEEIACTQNGQMYLNRDIWKPAPCQICVCDNGAILCDKIECQDVLDCADPVTPPGECCPVCSQTPGGGNTNFGRGRKGQKGEPGLVPVVTGIRGRPGPAGPPGSQGPRGERGPKGRPGPRGPQGIDGEPGVPGQPGAPGPPGHPSHPGPDGLSRPFSAQMAGLDEKSGLGSQVGLMPGSVGPVGPRGPQGLQGQQGGAGPTGPPGEPGDPGPMGPIGSRGPEGPPGKPGEDGEPGRNGNPGEVGFAGSPGARGFPGAPGLPGLKGHRGHKGLEGPKGEVGAPGSKGEAGPTGPMGAMGPLGPRGMPGERGRLGPQGAPGQRGAHGMPGKPGPMGPLGIPGSSGFPGNPGMKGEAGPTGARGPEGPQGQRGETGPPGPVGSPGLPGAIGTDGTPGAKGPTGSPGTSGPPGSAGPPGSPGPQGSTGPQGIRGQPGDPGVPGFKGEAGPKGEPGPHGIQGPIGPPGEEGKRGPRGDPGTVGPPGPVGERGAPGNRGFPGSDGLPGPKGAQGERGPVGSSGPKGSQGDPGRPGEPGLPGARGLTGNPGVQGPEGKLGPLGAPGEDGRPGPPGSIGIRGQPGSMGLPGPKGSSGDPGKPGEAGNAGVPGQRGAPGKDGEVGPSGPVGPPGLAGERGEQGPPGPTGFQGLPGPPGPPGEGGKPGDQGVPGDPGAVGPLGPRGERGNPGERGEPGITGLPGEKGMAGGHGPDGPKGSPGPSGTPGDTGPPGLQGMPGERGIAGTPGPKGDRGGIGEKGAEGTAGNDGARGLPGPLGPPGPAGPTGEKGEPGPRGLVGPPGSRGNPGSRGENGPTGAVGFAGPQGPDGQPGVKGEPGEPGQKGDAGSPGPQGLAGSPGPHGPNGVPGLKGGRGTQGPPGATGFPGSAGRVGPPGPAGAPGPAGPLGEPGKEGPPGLRGDPGSHGRVGDRGPAGPPGGPGDKGDPGEDGQPGPDGPPGPAGTTGQRGIVGMPGQRGERGMPGLPGPAGTPGKVGPTGATGDKGPPGPVGPPGSNGPVGEPGPEGPAGNDGTPGRDGAVGERGDRGDPGPAGLPGSQGAPGTPGPVGAPGDAGQRGDPGSRGPIGPPGRAGKRGLPGPQGPRGDKGDHGDRGDRGQKGHRGFTGLQGLPGPPGPNGEQGSAGIPGPFGPRGPPGPVGPSGKEGNPGPLGPIGPPGVRGSVGEAGPEGPPGEPGPPGPPGPPGHLTAALGDIMGHYDESMPDPLPEFTEDQAAPDDKNKTDPGVHATLKSLSSQIETMRSPDGSKKHPARTCDDLKLCHSAKQSGEYWIDPNQGSVEDAIKVYCNMETGETCISANPSSVPRKTWWASKSPDNKPVWYGLDMNRGSQFAYGDHQSPNTAITQMTFLRLLSKEASQNITYICKNSVGYMDDQAKNLKKAVVLKGANDLDIKAEGNIRFRYIVLQDTCSKRNGNVGKTVFEYRTQNVARLPIIDLAPVDVGGTDQEFGVEIGPVCFV

SEQ ID NO: 13 - protein, 1745 aa, Homo sapiens
MGNRRDLGQPRAGLCLLLAALQLLPGTQADPVDVLKALGVQGGQAGVPEGPGFCPQRTPEGDRAFRIGQASTLGIPTWELFPEGHFPENFSLLITLRGQPANQSVLLSIYDERGARQLGLALGPALGLLGDPFRPLPQQVNLTDGRWHRVAVSIDGEMVTLVADCEAQPPVLGHGPRFISIAGLTVLGTQDLGEKTFEGDIQELLISPDPQAAFQACERYLPDCDNLAPAATVAPQGEPETPRPRRKGKGKGRKKGRGRKGKGRKKNKEIWTSSPPPDSAENQTSTDIPKTETPAPNLPPTPTPLVVTSTVTTGLNATILERSLDPDSGTELGTLETKAAREDEEGDDSTMGPDFRAAEYPSRTQFQIFPGAGEKGAKGEPAVIEKGQQFEGPPGAPGPQGVVGPSGPPGPPGFPGDPGPPGPAGLPGIPGIDGIRGPPGTVIMMPFQFAGGSFKGPPVSFQQAQAQAVLQQTQLSMKGPPGPVGLTGRPGPVGLPGHPGLKGEEGAEGPQGPRGLQGPHGPPGRVGKMGRPGADGARGLPGDTGPKGDRGFDGLPGLPGEKGQRGDFGHVGQPGPPGEDGERGAEGPPGPTGQAGEPGPRGLLGPRGSPGPTGRPGVTGIDGAPGAKGNVGPPGEPGPPGQQGNHGSQGLPGPQGLIGTPGEKGPPGNPGIPGLPGSDGPLGHPGHEGPTGEKGAQGPPGSAGPPGYPGPRGVKGTSGNRGLQGEKGEKGEDGFPGFKGDVGLKGDQGKPGAPGPRGEDGPEGPKGQAGQAGEEGPPGSAGEKGKLGVPGLPGYPGRPGPKGSIGFPGPLGPIGEKGKSGKTGQPGLEGERGPPGSRGERGQPGATGQPGPKGDVGQDGAPGIPGEKGLPGLQGPPGFPGPKGPPGHQGKDGRPGHPGQRGELGFQGQTGPPGPAGVLGPQGKTGEVGPLGERGPPGPPGPPGEQGLPGLEGREGAKGELGPPGPLGKEGPAGLRGFPGPKGGPGDPGPTGLKGDKGPPGPVGANGSPGERGPLGPAGGIGLPGQSGSEGPVGPAGKKGSRGERGPPGPTGKDGIPGPLGPLGPPGAAGPSGEEGDKGDVGAPGHKGSKGDKGDAGPPGQPGIRGPAGHPGPPGADGAQGRRGPPGLFGQKGDDGVRGFVGVIGPPGLQGLPGPPGEKGEVGDVGSMGPHGAPGPRGPQGPTGSEGTPGLPGGVGQPGAVGEKGERGDAGDPGPPGAPGIPGPKGDIGEKGDSGPSGAAGPPGKKGPPGEDGAKGSVGPTGLPGDLGPPGDPGVSGIDGSPGEKGDPGDVGGPGPPGASGEPGAPGPPGKRGPSGHMGREGREGEKGAKGEPGPDGPPGRTGPMGARGPPGRVGPEGLRGIPGPVGEPGLLGAPGQMGPPGPLGPSGLPGLKGDTGPKGEKGHIGLIGLIGPPGEAGEKGDQGLPGVQGPPGPKGDPGPPGPIGSLGHPGPPGVAGPLGQKGSKGSPGSMGPRGDTGPAGPPGPPGAPAELHGLRRRRRFVPVPLPVVEGGLEEVLASLTSLSLELEQLRRPPGTAERPGLVCHELHRNHPHLPDGEYWIDPNQGCARDSFRVFCNFTAGGETCLYPDKKFEIVKLASWSKEKPGGWYSTFRRGKKFSYVDADGSPVNVVQLNFLKLLSATARQNFTYSCQNAAAWLDEATGDYSHSARFLGTNGEELSFNQTTAATVSVPQDGCRLRKGQTKTLFEFSSSRAGFLPLWDVAATDFGQTNQKFGFELGPVCFSS

SEQ ID NO: 14 - protein, 1019 aa, Homo sapiens
MLQGTCSVLLLWGILGAIQAQQQEVISPDTTERNNNCPEKTDCPIHVYFVLDTSESVTMQSPTDILLFHMKQFVPQFISQLQNEFYLDQVALSWRYGGLHFSDQVEVFSPPGSDRASFIKNLQGISSFRRGTFTDCALANMTEQIRQDRSKGTVHFAVVITDGHVTGSPCGGIKLQAERAREEGIRLFAVAPNQNLKEQGLRDIASTPHELYRNDYATMLPDSTEIDQDTINRIIKVMKHEAYGECYKVSCLEIPGPSGPKGYRGQKGAKGNMGEPGEPGQKGRQGDPGIEGPIGFPGPKGVPGFKGEKGEFGADGRKGAPGLAGKNGTDGQKGKLGRIGPPGCKGDPGNRGPDGYPGEAGSPGERGDQGGKGDPGRPGRRGPPGEIGAKGSKGYQGNSGAPGSPGVKGAKGGPGPRGPKGEPGRRGDPGTKGSPGSDGPKGEKGDPGPEGPRGLAGEVGNKGAKGDRGLPGPRGPQGALGEPGKQGSRGDPGDAGPRGDSGQPGPKGDPGRPGFSYPGPRGAPGEKGEPGPRGPEGGRGDFGLKGEPGRKGEKGEPADPGPPGEPGPRGPRGVPGPEGEPGPPGDPGLTECDVMTYVRETCGCCDCEKRCGALDVVFVIDSSESIGYTNFTLEKNFVINVVNRLGAIAKDPKSETGTRVGVVQYSHEGTFEAIQLDDERIDSLSSFKEAVKNLEWIAGGTWTPSALKFAYDRLIKESRRQKTRVFAVVITDGRHDPRDDDLNLRALCDRDVTVTAIGIGDMFHEKHESENLYSIACDKPQQVRNMTLFSDLVAEKFIDDMEDVLCPDPQIVCPDLPCQTELSVAQCTQRPVDIVFLLDGSERLGEQNFHKARRFVEQVARRLTLARRDDDPLNARVALLQFGGPGEQQVAFPLSHNLTAIHEALETTQYLNSFSHVGAGVVHAINAIVRSPRGGARRHAELSFVFLTDGVTGNDSLHESAHSMRKQNVVPTVLALGSDVDMDVLTTLSLGDRAAVFHEKDYDSLAQPGFFDRFIRWIC

SEQ ID NO: 15 - protein, 3177 aa, Homo sapiens
MRKHRHLPLVAVFCLFLSGFPTTHAQQQQADVKNGAAADIIFLVDSSWTIGEEHFQLVREFLYDVVKSLAVGENDFHFALVQFNGNPHTEFLLNTYRTKQEVLSHISNMSYIGGTNQTGKGLEYIMQSHLTKAAGSRAGDGVPQVIVVLTDGHSKDGLALPSAELKSADVNVFAIGVEDADEGALKEIASEPLNMHMFNLENFTSLHDIVGNLVSCVHSSVSPERAGDTETLKDITAQDSADIIFLIDGSNNTGSVNFAVILDFLVNLLEKLPIGTQQIRVGVVQFSDEPRTMFSLDTYSTKAQVLGAVKALGFAGGELANIGLALDFVVENHFTRAGGSRVEEGVPQVLVLISAGPSSDEIRYGVVALKQASVFSFGLGAQAASRAELQHIATDDNLVFTVPEFRSFGDLQEKLLPYIVGVAQRHIVLKPPTIVTQVIEVNKRDIVFLVDGSSALGLANFNAIRDFIAKVIQRLEIGQDLIQVAVAQYADTVRPEFYFNTHPTKREVITAVRKMKPLDGSALYTGSALDFVRNNLFTSSAGYRAAEGIPKLLVLITGGKSLDEISQPAQELKRSSIMAFAIGNKGADQAELEEIAFDSSLVFIPAEFRAAPLQGMLPGLLAPLRTLSGTPEVHSNKRDIIFLLDGSANVGKTNFPYVRDFVMNLVNSLDIGNDNIRVGLVQFSDTPVTEFSLNTYQTKSDILGHLRQLQLQGGSGLNTGSALSYVYANHFTEAGGSRIREHVPQLLLLLTAGQSEDSYLQAANALTRAGILTFCVGASQANKAELEQIAFNPSLVYLMDDFSSLPALPQQLIQPLTTYVSGGVEEVPLAQPESKRDILFLFDGSANLVGQFPVVRDFLYKIIDELNVKPEGTRIAVAQYSDDVKVESRFDEHQSKPEILNLVKRMKIKTGKALNLGYALDYAQRYIFVKSAGSRIEDGVLQFLVLLVAGRSSDRVDGPASNLKQSGVVPFIFQAKNADPAELEQIVLSPAFILAAESLPKIGDLHPQIVNLLKSVHNGAPAPVSGEKDVVFLLDGSEGVRSGFPLLKEFVQRVVESLDVGQDRVRVAVVQYSDRTRPEFYLNSYMNKQDVVNAVRQLTLLGGPTPNTGAALEFVLRNILVSSAGSRITEGVPQLLIVLTADRSGDDVRNPSVVVKRGGAVPIGIGIGNADITEMQTISFIPDFAVAIPTFRQLGTVQQVISERVTQLTREELSRLQPVLQPLPSPGVGGKRDVVFLIDGSQSAGPEFQYVRTLIERLVDYLDVGFDTTRVAVIQFSDDPKVEFLLNAHSSKDEVQNAVQRLRPKGGRQINVGNALEYVSRNIFKRPLGSRIEEGVPQFLVLISSGKSDDEVDDPAVELKQFGVAPFTIARNADQEELVKISLSPEYVFSVSTFRELPSLEQKLLTPITTLTSEQIQKLLASTRYPPPAVESDAADIVFLIDSSEGVRPDGFAHIRDFVSRIVRRLNIGPSKVRVGVVQFSNDVFPEFYLKTYRSQAPVLDAIRRLRLRGGSPLNTGKALEFVARNLFVKSAGSRIEDGVPQHLVLVLGGKSQDDVSRFAQVIRSSGIVSLGVGDRNIDRTELQTITNDPRLVFTVREFRELPNIEERIMNSFGPSAATPAPPGVDTPPPSRPEKKKADIVFLLDGSINFRRDSFQEVLRFVSEIVDTVYEDGDSIQVGLVQYNSDPTDEFFLKDFSTKRQIIDAINKVVYKGGRHANTKVGLEHLRVNHFVPEAGSRLDQRVPQIAFVITGGKSVEDAQDVSLALTQRGVKVFAVGVRNIDSEEVGKIASNSATAFRVGNVQELSELSEQVLETLHDAMHETLCPGVTDAAKACNLDVILGFDGSRDQNVFVAQKGFESKVDAILNRISQMHRVSCSGGRSPTVRVSVVANTPSGPVEAFDFDEYQPEMLEKFRNMRSQHPYVLTEDTLKVYLNKFRQSSPDSVKVVIHFTDGADGDLADLHRASENLRQEGVRALILVGLERVVNLERLMHLEFGRGFMYDRPLRLNLLDLDYELAEQLDNIAEKACCGVPCKCSGQRGDRGPIGSIGPKGIPGEDGYRGYPGDEGGPGERGPPGVNGTQGFQGCPGQRGVKGSRGFPGEKGEVGEIGLDGLDGEDGDKGLPGSSGEKGNPGRRGDKGPRGEKGERGDVGIRGDPGNPGQDSQERGPKGETGDLGPMGVPGRDGVPGGPGETGKNGGFGRRGPPGAKGNKGGPGQPGFEGEQGTRGAQGPAGPAGPPGLIGEQGISGPRGSGGAAGAPGERGRTGPLGRKGEPGEPGPKGGIGNRGPRGETGDDGRDGVGSEGRRGKKGERGFPGYPGPKGNPGEPGLNGTTGPKGIRGRRGNSGPPGIVGQKGDPGYPGPAGPKGNRGDSIDQCALIQSIKDKCPCCYGPLECPVFPTELAFALDTSEGVNQDTFGRMRDVVLSIVNDLTIAESNCPRGARVAVVTYNNEVTTEIRFADSKRKSVLLDKIKNLQVALTSKQQSLETAMSFVARNTFKRVRNGFLMRKVAVFFSNTPTRASPQLREAVLKLSDAGITPLFLTRQEDRQLINALQINNTAVGHALVLPAGRDLTDFLENVLTCHVCLDICNIDPSCGFGSWRPSFRDRRAAGSDVDIDMAFILDSAETTTLFQFNEMKKYIAYLVRQLDMSPDPKASQHFARVAVVQHAPSESVDNASMPPVKVEFSLTDYGSKEKLVDFLSRGMTQLQGTRALGSAIEYTIENVFESAPNPRDLKIVVLMLTGEVPEQQLEEAQRVILQAKCKGYFFVVLGIGRKVNIKEVYTFASEPNDVFFKLVDKSTELNEEPLMRFGRLLPSFVSSENAFYLSPDIRKQCDWFQGDQPTKNLVKFGHKQVNVPNNVTSSPTSNPVTTTKPVTTTKPVTTTTKPVTTTTKPVTIINQPSVKPAAAKPAPAKPVAAKPVATKMATVRPPVAVKPATAAKPVAAKPAAVRPPAAAAAKPVATKPEVPRPQAAKPAATKPATTKPMVKMSREVQVFEITENSAKLHWERAEPPGPYFYDLTVTSAHDQSLVLKQNLTVTDRVIGGLLAGQTYHVAVVCYLRSQVRATYHGSFSTKKSQPPPPQPARSASSSTINLMVSTEPLALTETDICKLPKDEGTCRDFILKWYYDPNTKSCARFWYGGCGGNENKFGSQKECEKVCAPVLAKPGVISVMGT

SEQ ID NO: 16 - protein, 2615 aa, Homo sapiens
MKILLIIFVLIIWTETLADQSPGPGPVYADVVFLVDSSDHLGPKSFPFVKTFINKMINSLPIEANKYRVALAQYSDEFHSEFHLSTFKGRSPMLNHLKKNFQFIGGSLQIGKALQEAHRTYFSAPINGRDRKQFPPILVVLASAESEDEVEEASKALQKDGVKIISVGVQKASEENLKAMATSHFHFNLRTIRDLSTFSQNMTQIIKDVTKYKEGAVDADMQVHFPISCQKDSLADLVFLVDESLGTGGNLRHLQTFLENITSSMDVKENCMRLGLMSYSNSAKTISFLKSSTTQSEFQQQIKNLSIQVGKSNTGAAIDQMRRDGFSESYGSRRAQGVPQIAVLVTHRPSDDEVHDAALNLRLEDVNVFALSIQGANNTQLEEIVSYPPEQTISTLKSYADLETYSTKFLKKLQNEIWSQISTYAEQRNLDKTGCVDTKEADIHFLIDGSSSIQEKQFEQIKRFMLEVTEMFSIGPDKVRVGVVQYSDDTEVEFYITDYSNDIDLRKAIFNIKQLTGGTYTGKALDYILQIIKNGMKDRMSKVPCYLIVLTDGMSTDRVVEPAKRLRAEQITVHAVGIGAANKIELQEIAGKEERVSFGQNFDALKSIKNEVVREICAEKGCEDMKADIMFLVDSSWSIGNENFRKMKIFMKNLLTKIQIGADKTQIGVVQFSDKTKEEFQLNRYFTQQEISDAIDRMSLINEGTLTGKALNFVGQYFTHSKGARLGAKKFLILITDGVAQDDVRDPARILRGKDVTIFSVGVYNANRSQLEEISGDSSLVFHVENFDHLKALERKLIFRVCALHDCKRITLLDVVFVLDHSGSIKKQYQDHMINLTIHLVKKADVGRDRVQFGALKYSDQPNILFYLNTYSNRSAIIENLRKRRDTGGNTYTAKALKHANALFTEEHGSRIKQNVKQMLIVITDGESHDHDQLNDTALELRNKGITIFAVGVGKANQKELEGMAGNKNNTIYVDNFDKLKDVFTLVQERMCTEAPEVCHLQEADVIFLCDGSDRVSNSDFVTMTTFLSDLIDNFDIQSQRMKIGMAQFGSNYQSIIELKNSLTKTQWKTQIQNVSKSGGFPRIDFALKKVSNMFNLHAGGRRNAGVPQTLVVITSGDPRYDVADAVKTLKDLGICVLVLGIGDVYKEHLLPITGNSEKIITFQDFDKLKNVDVKKRIIREICQSCGKTNCFMDIVVGFDISTHVQGQPLFQGHPQLESYLPGILEDISSIKGVSCGAGTEAQVSLAFKVNSDQGFPAKFQIYQKAVFDSLLQVNVSGPTHLNAQFLRSLWDTFKDKSASRGQVLLIFSDGLQSESNIMLENQSDRLREAGLDALLVVSLNTTAHHEFSSFEFGKRFDYRTHLTIGMRELGKKLSQYLGNIAERTCCCTFCKCPGIPGPHGTRGLQAMKGSQGLKGSRGHRGEDGNPGVRGDTGPQGDKGIAGCPGAWGQKGLKGFSGPKGGHGDDGIDGLDGEEGCHGFPGIKGEKGDPGSQGSPGSRGAPGQYGEKGFPGDPGNPGQNNNIKGQKGSKGEQGRQGRSGQKGVQGSPSSRGSRGREGQRGLRGVSGEPGNPGPTGTLGAEGLQGPQGSQGNPGRKGEKGSQGQKGPQGSPGLMGAKGSTGRPGLLGKKGEPGLPGDLGPVGQTGQRGRQGDSGIPGYGQMGRKGVKGPRGFPGDAGQKGDIGNPGIPGGPGPKGFRGLALTVGLKGEEGSRGLPGPPGQRGIKGMAGQPVYSQCDLIRFLREHSPCWKEKCPAYPTELVFALDNSYDVTEESFNKTRDIITSIVNDLNIRENNCPVGARVAMVSYNSGTSYLIRWSDYNRKKQLLQQLSQIKYQDTTEPRDVGNAMRFVTRNVFKRTYAGANVRRVAVFFSNGQTASRSSIITATMEFSALDISPTVFAFDERVFLEAFGFDNTGTFQVIPVPPNGENQTLERLRRCALCYDKCFPNACIREAFLPEDSYMDVVFLIDNSRNIAKDEFKAVKALVSSVIDNFNIASDPLISDSGDRIALLSYSPWESSRRKMGTVKTEFDFITYDNQLLMKNHIQTSFQQLNGEATIGRALLWTTENLFPETPYLRKHKVIFVVSAGENYERKEFVKMMALRAKCQGYVIFVISLGSTRKDDMEELASYPLDQHLIQLGRIHKPDLNYIAKFLKPFLYSVRRGFNQYPPPMLEDACRLINLGGENIQNDGFQFVTELQEDFLGGNGFIGQELNSGRESPFVKTEDNGSDYLVYLPSQMFEPQKLMINYEKDQKSAEIASLTSGHENYGRKEEPDHTYEPGDVSLQEYYMDVAFLIDASQRVGSDEFKEVKAFITSVLDYFHIAPTPLTSTLGDRVAVLSYSPPGYMPNTEECPVYLEFDLVTYNSIHQMKHHLQDSQQLNGDVFIGHALQWTIDNVFVGTPNLRKNKVIFVISAGETNSLDKDVLRNVSLRAKCQGYSIFVFSFGPKHNDKELEELASHPLDHHLVQLGRTHKPDWNYIIKFVKPFVHLIRRAINKYPTEDMKATCVNMTSPNPENGGTENTVLLLPGIYEIKTENGDLFDEFDSQAQHLLVLGNNHSSGSETATDLMQKLYLLFSTEKLAMKDKEKAHLEEISALVVDKQQEKEDKEMEATDI

SEQ ID NO: 17 - protein, 2263 aa, Homo sapiens
MMLLILFLVIICSHISVNQDSGPEYADVVFLVDSSDRLGSKSFPFVKMFITKMISSLPIEADKYRVALAQYSDKLHSEFHLSTFKGRSPMLNHLRKNFGFIGGSLQIGKALQEAHRTYFSAPANGRDKKQFPPILVVLASSESEDNVEEASKALRKDGVKIISVGVQKASEENLKAMATSQFHFNLRTVRDLSMFSQNMTHIIKDVIKYKEGAVDDIFVEACQGPSMADVVFLLDMSINGSEENFDYLKGFLEESVSALDIKENCMRVGLVAYSNETKVINSLSMGINKSEVLQHIQNLSPRTGKAYTGAAIKKLRKEVFSARNGSRKNQGVPQIAVLVTHRDSEDNVTKAAVNLRREGVTIFTLGIEGASDTQLEKIASHPAEQYVSKLKTFADLAAHNQTFLKKLRNQITHTVSVFSERTETLKSGCVDTEEADIYLLIDGSGSTQATDFHEMKTFLSEVVGMFNIAPHKVRVGAVQYADSWDLEFEINKYSNKQDLGKAIENIRQMGGNTNTGAALNFTLSLLQKAKKQRGNKVPCHLVVLTNGMSKDSILEPANRLREEHIRVYAIGIKEANQTQLREIAGEEKRVYYVHDFDALKDIRNQVVQEICTEEACKEMKADIMFLVDSSGSIGPENFSKMKTFMKNLVSKSQIGPDRVQIGVVQFSDINKEEFQLNRFMSQSDISNAIDQMAHIGQTTLTGSALSFVSQYFSPTKGARPNIRKFLILITDGEAQDIVKEPAVVLRQEGVIIYSVGVFGSNVTQLEEISGRPEMVFYVENFDILQRIEDDLVFGICSPREECKRIEVLDVVFVIDSSGSIDYDEYNIMKDFMIGLVKKADVGKNQVRFGALKYADDPEVLFYLDDFGTKLEVISVLQNDQAMGGSTYTAEALGFSDHMFTEARGSRLNKGVPQVLIVITDGESHDADKLNATAKALRDKGILVLAVGIDGANPVELLAMAGSSDKYFFVETFGGLKGIFSDVTASVCNSSKVDCEIDKVDLVFLMDGSTSIQPNDFKKMKEFLASVVQDFDVSLNRVRIGAAQFSDTYHPEFPLGTFIGEKEISFQIENIKQIFGNTHIGAALREVEHYFRPDMGSRINTGTPQVLLVLTDGQSQDEVAQAAEALRHRGIDIYSVGIGDVDDQQLIQITGTAEKKLTVHNFDELKKVNKRIVRNICTTAGESNCFVDVVVGFDVSTQEKGQTLLEGQPWMETYLQDILRAISSLNGVSCEVGTETQVSVAFQVTNAMEKYSPKFEIYSENILNSLKDITVKGPSLLNANLLDSLWDTFQNKSAARGKVVLLFSDGLDDDVEKLEQKSDELRKEGLNALITVALDGPADSSDLADLPYIEFGKGFEYRTQLSIGMRELGSRLSKQLVNVAERTCCCLFCKCIGGDGTMGDPGPPGKRGPPGFKGSEGYLGEEGIAGERGAPGPVGEQGTKGCYGTKGPKGNRGLNGQEGEVGENGIDGLNGEQGDNGLPGRKGEKGDEGSQGSPGKRGTPGDRGAKGLRGDPGAPGVDSSIEGPTGLKGERGRQGRRGWPGPPGTPGSRRKTAAHGRRGHTGPQGTAGIPGPDGLEGSLGLKGPQGPRGEAGVKGEKGGVGSKGPQGPPGPGGEAGNQGRLGSQGNKGEPGDLGEKGAVGFPGPRGLQGNDGSPGYGSVGRKGAKGQEGFPGESGPKGEIGDPGGPGETGLKGARGKMISAGLPGEMGSPGEPGPPGRKGVKGAKGLASFSTCELIQYVRDRSPGRHGKPECPVHPTELVFALDHSRDVTEQEFERMKEMMAFLVRDIKVRENSCPVGAHIAILSYNSHARHLVRFSDAYKKSQLLREIETIPYERSSASREIGRAMRFISRNVFKRTLPGAHTRKIATFFSSGQSADAHSITTAAMEFGALEIIPVVITFSNVPSVRRAFAIDDTGTFQVIVVPSGADYIPALERLQRCTFCYDVCKPDASCDQARPPPVQSYMDAAFLLDASRNMGSAEFEDIRAFLGALLDHFEITPEPETSVTGDRVALLSHAPPDFLPNTQKSPVRAEFNLTTYRSKRLMKRHVHESVKQLNGDAFIGHALQWTLDNVFLSTPNLRRNKVIFVISAGETSHLDGEILKKESLRAKCQGYALFVFSLGPIWDDKELEDLASHPLDHHLVQLGRIHKPDHSYGVKFVKSFINSIRRAINKYPPINLKIKCNRLNSIDPKQPPRPFRSFVPGPLKATLKEDVLQKAKFFQDKKYLSRVARSGRDDAIQNFMRSTSHTFKNGRMIESAPKQHD

(19) United States
(12) Patent Application Publication
(10) Pub. No.: US 2024/0150717 A1
(43) Pub. Date: May 9, 2024

(54) METHODS AND COMPOSITIONS FOR MANUFACTURING EXTRACELLULAR MATRIX

(71) Applicant: Breakthrough Tech LLC, Aliso Viejo, CA (US)

(72) Inventor: Francis Christopher ZEIGLER, Aliso Viejo, CA (US)

(21) Appl. No.: 18/335,001

(22) Filed: Jun. 14, 2023

Related U.S. Application Data
(63) Continuation of application No. 15/662,901, filed on Jul. 28, 2017, now Pat. No. 11,718,829.

(51) Int. Cl. C12N 5/077; C12N 5/074

(52) U.S. Cl. CPC C12N 5/0656; C12N 5/0696; C12N 2501/10; C12N 2506/1307; C12N 2506/45

(57) ABSTRACT

Embodiments herein include methods, kits, and compositions for manufacturing extracellular matrix (ECM). In some embodiments, the methods comprise differentiating fibroblasts into induced pluripotent stem cells, expanding the induced pluripotent stem cells, and differentiating the induced pluripotent stem cells into fibroblasts. The fibroblasts can produced mature ECM, which can be isolated and used for medical and/or cosmetic products and procedures.

### REFERENCE TO SEQUENCE LISTING, TABLE, OR COMPUTER PROGRAM LISTING

The present application is being filed along with a Sequence Listing in electronic format. The Sequence Listing is provided as a file entitled SEQUENCEWYLD008A.TXT, created and last modified Jul. 18, 2017, which is 247,512 bytes in size. The information in the electronic format of the Sequence Listing is incorporated herein by reference in its entirety.

### BACKGROUND

The extracellular matrix (ECM) is a versatile biomaterial with many cosmetic and therapeutic uses. The majority of connective tissues comprise collagens, and to a much lesser extent (based on relative abundance by weight) other glycoproteins such as laminins, fibronectin, and glycosaminoglycans (GAGs) including hyaluronic acid, and other sulfated GAGs such as aggrecan and perlecan. However, collagens are cross-linked and require enzymatic or chemical degradation to isolate for cosmetic or therapeutic uses and manufacture into useful products for human use. Examples including the use of bovine and porcine corium after pepsin digestion or chemical modification, porcine intestinal submucosa, and human cadaver-derived tissues such as skin and bone, are all widely known in the art.

Animal sources for ECM such as porcine and bovine tissues carry the risk of unwanted immune reactions, including known allergies to bovine and porcine antigens or allergens, mostly proteins, while human cells that use animal-derived components (including most often bovine serum, bovine albumin, or porcine trypsin), or non-human plant-derived proteins (including soybean trypsin inhibitor or recombinant human albumin produced in plants which can contain residual plant proteins or polypeptides) suffer from the same risks. Accordingly, the commercial usefulness of ECM from animal sources can be limited by safety concerns and/or regulatory hurdles for validating the animal components' removal to safer residual levels needed for approval to use commercially. The human immune system is sensitive enough to react to extremely low abundance antigens, and animal and plant proteins have been demonstrated to cause unwanted immune reactions, including potentially life-threatening allergic reactions which can cause anaphylactic shock and even death in some cases. Animal-derived and plant-derived protein components, and human cells grown in animal-derived or plant-derived protein components are collectively termed “xenogeneic.” On the other hand, products manufactured without contact to these animal-derived and plant-derived protein components are known as “xeno-free.” Conventional approaches for manufacturing ECM in xenogenic media can require removal of animal-derived and/or plant-derived protein components, limiting the commercial usefulness of these methods.

Human ECM can be derived from allogeneic tissues from cadavers. However, such cadaver-derived ECM presents risk of disease transmission, as well as limited commercial scalability, since each donor provides limited amounts of obtainable human ECM (e.g., a 70 kg human contains less than 20% by weight human collagens or no greater than a few kgs raw ECM materials). Additionally, cadaver-derived tissues involve expensive safety testing to mitigate some risk of disease transmission, and must be extensively tested for a number of disease-causing pathogens including viruses and bacteria for each single cadaverous donor.

Additionally, ECM can be used for animal feed. However, as a practical matter, conventional approaches for producing ECM using cell culture or harvest from cadavers can be cost-prohibitive on a commercial scale. Described in accordance with some embodiments herein are methods, compositions, and kits for efficiently producing ECM at a commercial scale, while minimizing risks of disease-transmission and immunogenicity.

### FIELD

Some embodiments herein relate to methods and compositions for manufacturing extracellular matrix (ECM). In some embodiments, methods and compositions for cell culture are described.

### SUMMARY

Some embodiments include a method of manufacturing extracellular matrix. The method can comprise differentiating induced Pluripotent Stem Cells (iPSCs) into production fibroblast. The method can comprise culturing the production fibroblasts, and by way of the culturing, the production fibroblasts can produce extracellular matrix (ECM). The method can comprise isolating the ECM from the production fibroblasts, thus manufacturing the ECM. In some embodiments, the method further comprises de-differentiating a precursor fibroblast to form the iPSCs prior to differentiating the iPSCs into the production fibroblast. In some embodiments, the method further comprises expanding the iPSC's prior to the differentiating, for example at least about 2, 3, 4, 5, 6, 7, 8, 9, 10, 11, 12, 13, 14, 15, 16, 17, 18, 19, 20, 25, or 30 doublings, including ranges between any two of the listed values. In some embodiments, the method further comprises constructing a bank of the IBC's prior to the differentiating. In some embodiments, only iPSCS are differentiated are from a single donor. In some embodiments, the culturing of the production fibroblasts is in normoxia. In some embodiments, culturing the production fibroblasts does not comprise culturing mesenchymal stem cells (MSCs). In some embodiments, the precursor fibroblast comprises an adult dermal (biopsy) fibroblast In some embodiments, isolating the ECM comprises purifying the ECM, thereby manufacturing a composition that is at least about 80% w/w ECM. In some embodiments, purifying the ECM comprises washing the ECM in an acidic buffer, and contacting a solution comprising the production fibroblast and ECM with dextranase. In some embodiments, the ECM comprises collagen. In some embodiments, about 90% (w/w) of the ECM is COL1, and about 10% is selected from the group consisting of: COL3, COL4, COL5, COL6, and a combination of any of these. In some embodiments, the method further comprises contacting the precursor fibroblast with de-differentiation factors, thereby de-differentiating the precursor fibroblast into the iPSC. In some embodiments, the iPSCs are free of viral insertions encoding an Oct family member, a Sox family member, a Klf family member. In some embodiments, the iPSCs are footprint-free. In some embodiments, the method does not comprise any of: an embryonic stem (ES) cell, a bone marrow multipotent stem cell, an ES-derived MSC, or a non-multipotent neonatal foreskin fibroblast cell line. In some embodiments, the ECM comprises a c-terminal propeptide of COL1, or a triple-helical or non-reducible gamma-form fibrillar collagen, or both. In some embodiments, the method further comprises contacting the production fibroblasts with serum, until the production fibroblasts produce mature collagens, and then gradually reducing the amount of serum until there is a at least 95% reduction in the concentration of serum. In some embodiments, the gradual reducing is over a period of at least about 5 days.

Some embodiments include a kit for manufacturing ECM. The kit can comprise a composition comprising human fibroblasts. The kit can comprise de-differentiation factors. The kit can comprise fibroblast differentiation factors. In some embodiments, all of the fibroblasts of the composition are from a single donor. In some embodiments, the kit further comprises a substrate, such as dextran microcarriers. In some embodiments, the kit further comprises dextranase and DNAase.

Some embodiments include a composition comprising at least 80% (w w) extracellular matrix, in which the extracellular matrix is manufactured according to any of the above methods.

Some embodiments include a cell culture comprising iPSC-derived fibroblasts, in which the iPSC-derived fibroblasts are producing mature extracellular matrix. The cell culture can further include de-differentiation factors. In some embodiments, at least 50% of the composition (w/w) comprises ECM.

Some embodiments include a method of manufacturing extracellular matrix (ECM). The method can comprise culturing fibroblasts and/or mesenchymal stem cells (MSCs) on a substrate. The substrate can comprise at least two surfaces. The culturing can be serum-free and xeno-free. The culturing can be performed until the fibroblasts and/or MSCs define a three-dimensional shape over the at least two-surfaces and at least 80% of fibroblasts and/or MSCs arrest their cell cycle. The method can then include contacting the fibroblasts and/or MSCs with serum for at least about two weeks, through which the fibroblasts and/or MSCs produce soluble mature ECM. Thus, a solution comprising soluble mature ECM and the fibroblasts or MSCs can be produced, in which the solution is xeno-free. The method can further include isolating the soluble mature ECM from the production fibroblast, thus manufacturing the ECM, wherein the ECM is mature xeno-free ECM. In some embodiments, the method further comprises expanding a human pluripotent cell culture, the expanding being serum-free and xeno-free, thereby producing human pluripotent cells; and contacting the human pluripotent cells with differentiation factors, though which the human pluripotent cells differentiate into the fibroblasts or MSCs. In some embodiments, the contacting of the fibroblasts and/or MSCs with serum is for about 2 weeks to about 8 weeks. In some embodiments, the contacting of the fibroblasts and/or MSCs with serum is for at least about 8 weeks. In some embodiments, the human pluripotent cells comprise induced pluripotent stem cells (iPSCSs). In some embodiments, the iPSCSs are footprint-free. In some embodiments, the iPSCSs are from a single donor. In some embodiments, the method further comprises manufacturing a cosmetic composition comprising the mature xeno-free ECM. In some embodiments, the method further comprises contacting the fibroblasts or MSCs with ascorbic acid during the at least two weeks of the contacting with the serum. In some embodiments, the fibroblasts or MSCs are not contacted with serum prior to said fibroblasts or MSCs over the at least two-surfaces defining a three-dimensional shape and at least 70% of the fibroblasts or MSCs arresting their cell cycle. In some embodiments, the amount of serum is about 0.1% to 10% (v/v). In some embodiments, the amount of serum is about 1-2% (v/v). In some embodiments, the serum comprises clinical-grade bovine calf serum, pooled human serum, or a combination thereof. In some embodiments, pluripotent cells are of a cell line that was previously grown using animal components. In some embodiments, the mature xeno-free ECM comprises fibrillar collagen. In some embodiments, the mature xeno-free ECM comprises a c-terminal propeptide of COL1, or a triple-helical or non-reducible gamma-form fibrillar collagen, or both. In some embodiments, the solution comprises at least 250 μg of collagen per cm² of the substrate. In some embodiments, the manufactured mature xeno-free ECM comprises at least 250 μg of collagen per cm² of the substrate. In some embodiments, the pluripotent cells are from a single donor. In some embodiments, the method further comprises detecting an amount of mature ECM in the solution. In some embodiments, the method further comprises collecting a quantity of spent culture medium from the solution and isolating soluble mature ECM from the spent culture medium.

Some embodiments include a solution comprising fibroblasts or MSCs and the soluble mature ECM produced according to any of the methods of the above paragraph. The solution can be xeno-free, and the soluble mature ECM can comprise cross-linked collagen.

Some embodiments include a method of manufacturing extracellular matrix (ECM). The method can comprise providing fibroblasts in a medium comprising a concentration of serum. The method can comprise gradually reducing the amount of serum in the medium comprising fibroblasts until the medium contains no more than 5% of the concentration of serum. The method can comprise, following the gradually reduction of serum, culturing the fibroblasts for at least about 2 weeks, through which the fibroblasts produce soluble ECM, thereby producing a solution comprising the fibroblasts and soluble ECM. The method can comprise isolating the soluble ECM from the fibroblasts, thereby manufacturing the ECM. In some embodiments, a quantity of fibroblasts in the medium at the start of gradually reducing the amount of serum is at least 0.7× of a quantity of fibroblasts in the medium when the medium contains no more than 5% of the concentration in serum. In some embodiments, the quantity of fibroblasts in the medium at the start of gradually reducing the amount of serum is at least 0.9× of the quantity of fibroblasts in the medium when the medium contains no more than 5% of the concentration in serum. In some embodiments, gradually reducing the amount of serum is done without cell expansion or cell subculture. In some embodiments, the serum is gradually reduced for at least about 5 days. In some embodiments, at least about 90% of the fibroblasts in the solution are in a G₀ cell cycle phase. In some embodiments, fewer than 1% of the fibroblasts in the solution are undergoing apoptosis. In some embodiments, the solution comprises nanostructures comprising the soluble ECM, said nanostructures having a greatest diameter of 200 nm to 10,000 nm. In some embodiments, manufacturing the ECM is performed without sterile-filtering. (so as not to exclude the nanostructures). In some embodiments, isolating the soluble ECM from the fibroblasts is performed without sterile-filtering.

Some embodiments include a solution comprising fibroblasts and soluble ECM. At least about 90% of the fibroblasts in the solution can be in a G₀ cell cycle phase. Fewer than 1% of the fibroblasts in the solution can be undergoing apoptosis. The solution can comprise nanostructures comprising the soluble ECM, said nanostructures having a greatest diameter of 200 nm to 10,000 nm. In some embodiments, the soluble ECM is manufactured according to a method of any one of claims 48-57.

### BRIEF DESCRIPTION OF THE DRAWINGS

FIG. 1 is a flow diagram illustrating methods of manufacturing ECM using fibroblasts differentiated from iPSCs in accordance with some embodiments herein.

FIG. 2 is a flow diagram illustrating methods of manufacturing ECM comprising culturing fibroblasts or MSCs for at least two weeks in accordance with some embodiments herein.

FIG. 3 is a flow diagram illustrating methods of manufacturing ECM comprising serum weaning in accordance with some embodiments herein. The ECM can be xeno-free.

### DETAILED DESCRIPTION

ECM can be useful for a number of cosmetic and therapeutic applications, but conventional methods of producing ECM can suffer from challenges related to scalability, ease of use, contamination (including immunogenicity in human hosts), disease transmission, and cost efficiency. Described herein are methods, compositions, and kits useful for efficiently manufacturing ECM, including at commercial scales. The ECM in accordance with methods, compositions, and kits of some embodiments herein can comprise a relatively high level of commercially-useful mature collagens (such as triple-helical or non-reducible gamma-form fibrillar collagen, or both), and can be relatively free of contaminants such as potentially harmful xeno substances.

Some embodiments include methods, compositions, and kits for making non-embryonic human fibroblasts for use as a bioreactor for manufacturing human extracellular matrix (ECM). In accordance with these embodiments, mature fibroblasts can be de-differentiated into induced pluripotent stem cells (iPSCs), expanded, and re-differentiated into mature non-embryonic production fibroblasts. These production fibroblasts can be used to manufacture collagen-rich ECM.

Some embodiments include methods, compositions, and/or kits for manufacturing mature xeno-free ECM. In accordance with these embodiments, cells can be cultured for an extended period of time (e.g., up to 8 weeks, such as over 2-3 weeks or more), which can yield ECM with desirable characteristics such as increased cross-linking of collagen and superior ECM solubility. In contrast, many conventional approaches for manufacturing ECM may perform cell culture for a much shorter time, for example 12-17 days. Surprisingly, the 2-3 week or more cell cultures in accordance with some embodiments herein produce ECM with increased cross-linking and solubility compared to cultures of shorter periods of time.

Some embodiments include methods of culturing fibroblasts to produce ECM in which the fibroblasts are gradually weaned off of serum. In accordance with these embodiments, fibroblasts can first be cultured in a serum-containing formulation, and the amount of serum can then gradually be reduced (for example, by removing and replacing culture medium so as to lower serum content and/or by moving the cells to different culture medium with lower serum content). The cells can produce mature soluble ECM for at least 2 weeks following the reduction of the serum, and the soluble ECM can be isolated from the cells. “Soluble” (for example in the context of soluble ECM), is used herein in accordance with its ordinary meaning in the field, and includes a type or fraction of ECM which is dissolved or can be dissolved in an aqueous phase. As such, soluble ECM can recovered in an aqueous phase and maintained in an aqueous phase. In some embodiments, the soluble ECM does not precipitate in an aqueous phase. In some embodiments, the soluble ECM can be maintained stably in an aqueous phase under the same conditions for at least 24 hours, with minimal precipitation, for example so that about or less than about 10%, 9%, 8%, 7%, 6%, 5%, 4%, 3%, 2%, or 1%, of the soluble ECM precipitates. This method can advantageously yield highly-soluble ECM, which can be useful for cosmetic and medical products. In some embodiments, ECM is produced efficiently without any cell expansion or subculturing. It is noted that in some embodiments, both soluble and non-soluble ECM can be recovered from the same culture, advantageously recovering ECM in both phases and obtaining higher yield than a method that only obtains ECM from one of the phases.

### Extracellular Matrix (ECM)

“Extracellular Matrix” (ECM) is used herein in accordance with its ordinary meaning in the field, and includes molecules secreted by cells such as proteins and carbohydrates, which provide a structure to support the cells. The ECM can comprise fibrillar proteins such as collagen. Human ECM can include a number of collagen proteins, including, for example COL1, COL3, COL4, COL5, and COL6, as well as combinations of these proteins. Example polypeptide sequences of Homo sapiens COL1, COL3, COL4, COL5, and COL6 are shown in Table 1, below. In methods, compositions, and/or kits of some embodiments, cell cultures produce ECM, which can be isolated from the cell cultures. In some embodiments, the ECM comprises, consists essentially of, or consists of human ECM. In some embodiments, isolating the ECM from cell cultures comprises purifying the ECM. Human ECM products comprising, consisting essentially of, or consisting of the isolated ECM can thus be produced.

TABLE 1
Example Homo sapiens collagen polypeptide sequences

| Gene Name | | | SEQ |
|---|---|---|---|
| (Protein Name) | Uniprot ID | Polypeptide Sequence | ID NO: |
| COL1A1 | P02452 | MFSFVDLRLLLLLAATALLTHGQEEGQVEGQDEDIPPITCV | 1 |
| (Collagen | | QNGLRYHDRDVWKPEPCRICVCDNGKVLCDDVICDETKNCP | |
| alpha-1(I) | | GAEVPEGECCPVCPDGSESPTDQETTGVEGPKGDTGPRGPR | |
| chain) | | GPAGPPGRDGIPGQPGLPGPPGPPGPPGPPGLGGNFAPQLS | |
| | | YGYDEKSTGGISVPGPMGPSGPRGLPGPPGAPGPQGFQGPP | |
| | | GEPGEPGASGPMGPRGPPGPPGKNGDDGEAGKPGRPGERGP | |
| | | PGPQGARGLPGTAGLPGMKGHRGFSGLDGAKGDAGPAGPKG | |
| | | EPGSPGENGAPGQMGPRGLPGERGRPGAPGPAGARGNDGAT | |
| | | GAAGPPGPTGPAGPPGFPGAVGAKGEAGPQGPRGSEGPQGV | |
| | | RGEPGPPGPAGAAGPAGNPGADGQPGAKGANGAPGIAGAPG | |
| | | FPGARGPSGPQGPGGPPGPKGNSGEPGAPGSKGDTGAKGEP | |
| | | GPVGVQGPPGPAGEEGKRGARGEPGPTGLPGPPGERGGPGS | |
| | | RGFPGADGVAGPKGPAGERGSPGPAGPKGSPGEAGRPGEAG | |
| | | LPGAKGLTGSPGSPGPDGKTGPPGPAGQDGRPGPPGPPGAR | |
| | | GQAGVMGFPGPKGAAGEPGKAGERGVPGPPGAVGPAGKDGE | |
| | | AGAQGPPGPAGPAGERGEQGPAGSPGFQGLPGPAGPPGEAG | |
| | | KPGEQGVPGDLGAPGPSGARGERGFPGERGVQGPPGPAGPR | |
| | | GANGAPGNDGAKGDAGAPGAPGSQGAPGLQGMPGERGAAGL | |
| | | PGPKGDRGDAGPKGADGSPGKDGVRGLTGPIGPPGPAGAPG | |
| | | DKGESGPSGPAGPTGARGAPGDRGEPGPPGPAGFAGPPGAD | |
| | | GQPGAKGEPGDAGAKGDAGPPGPAGPAGPPGPIGNVGAPGA | |
| | | KGARGSAGPPGATGFPGAAGRVGPPGPSGNAGPPGPPGPAG | |
| | | KEGGKGPRGETGPAGRPGEVGPPGPPGPAGEKGSPGADGPA | |
| | | GAPGTPGPQGIAGQRGVVGLPGQRGERGFPGLPGPSGEPGK | |
| | | QGPSGASGERGPPGPMGPPGLAGPPGESGREGAPGAEGSPG | |
| | | RDGSPGAKGDRGETGPAGPPGAPGAPGAPGPVGPAGKSGDR | |
| | | GETGPAGPTGPVGPVGARGPAGPQGPRGDKGETGEQGDRGI | |
| | | KGHRGFSGLQGPPGPPGSPGEQGPSGASGPAGPRGPPGSAG | |
| | | APGKDGLNGLPGPIGPPGPRGRTGDAGPVGPPGPPGPPGPP | |
| | | GPPSAGFDFSFLPQPPQEKAHDGGRYYRADDANVVRDRDLE | |
| | | VDTTLKSLSQQIENIRSPEGSRKNPARTCRDLKMCHSDWKS | |
| | | GEYWIDPNQGCNLDAIKVFCNMETGETCVYPTQPSVAQKNW | |
| | | YISKNPKDKRHVWFGESMTDGFQFEYGGQGSDPADVAIQLT | |
| | | FLRLMSTEASQNITYHCKNSVAYMDQQTGNLKKALLLQGSN | |
| | | EIEIRAEGNSRFTYSVTVDGCTSHTGAWGKTVIEYKTTKTS | |
| | | RLPIIDVAPLDVGAPDQEFGFDVGPVCFL | |
| COL1A2 | P08123 | MLSFVDTRTLLLLAVTLCLATCQSLQEETVRKGPAGDRGPR | 2 |
| Collagen | | GERGPPGPPGRDGEDGPTGPPGPPGPPGPPGLGGNFAAQYD | |
| alpha-2(I) | | GKGVGLGPGPMGLMGPRGPPGAAGAPGPQGFQGPAGEPGEP | |
| chain | | GQTGPAGARGPAGPPGKAGEDGHPGKPGRPGERGVVGPQGA | |
| | | RGFPGTPGLPGFKGIRGHNGLDGLKGQPGAPGVKGEPGAPG | |
| | | ENGTPGQTGARGLPGERGRVGAPGPAGARGSDGSVGPVGPA | |
| | | GPIGSAGPPGFPGAPGPKGEIGAVGNAGPAGPAGPRGEVGL | |
| | | PGLSGPVGPPGNPGANGLTGAKGAAGLPGVAGAPGLPGPRG | |
| | | IPGPVGAAGATGARGLVGEPGPAGSKGESGNKGEPGSAGPQ | |
| | | GPPGPSGEEGKRGPNGEAGSAGPPGPPGLRGSPGSRGLPGA | |
| | | DGRAGVMGPPGSRGASGPAGVRGPNGDAGRPGEPGLMGPRG | |
| | | LPGSPGNIGPAGKEGPVGLPGIDGRPGPIGPAGARGEPGNI | |
| | | GFPGPKGPTGDPGKNGDKGHAGLAGARGAPGPDGNNGAQGP | |
| | | PGPQGVQGGKGEQGPPGPPGFQGLPGPSGPAGEVGKPGERG | |
| | | LHGEFGLPGPAGPRGERGPPGESGAAGPTGPIGSRGPSGPP | |
| | | GPDGNKGEPGVVGAVGTAGPSGPSGLPGERGAAGIPGGKGE | |
| | | KGEPGLRGEIGNPGRDGARGAPGAVGAPGPAGATGDRGEAG | |
| | | AAGPAGPAGPRGSPGERGEVGPAGPNGFAGPAGAAGQPGAK | |
| | | GERGAKGPKGENGVVGPTGPVGAAGPAGPNGPPGPAGSRGD | |
| | | GGPPGMTGFPGAAGRTGPPGPSGISGPPGPPGPAGKEGLRG | |
| | | PRGDQGPVGRTGEVGAVGPPGFAGEKGPSGEAGTAGPPGTP | |
| | | GPQGLLGAPGILGLPGSRGERGLPGVAGAVGEPGPLGIAGP | |
| | | PGARGPPGAVGSPGVNGAPGEAGRDGNPGNDGPPGRDGQPG | |
| | | HKGERGYPGNIGPVGAAGAPGPHGPVGPAGKHGNRGETGPS | |
| | | GPVGPAGAVGPRGPSGPQGIRGDKGEPGEKGPRGLPGLKGH | |
| | | NGLQGLPGIAGHHGDQGAPGSVGPAGPRGPAGPSGPAGKDG | |
| | | RTGHPGTVGPAGIRGPQGHQGPAGPPGPPGPPGPPGVSGGG | |
| | | YDFGYDGDFYRADQPRSAPSLRPKDYEVDATLKSLNNQIET | |
| | | LLTPEGSRKNPARTCRDLRLSHPEWSSGYYWIDPNQGCTMD | |
| | | AIKVYCDFSTGETCIRAQPENIPAKNWYRSSKDKKHVWLGE | |
| | | TINAGSQFEYNVEGVTSKEMATQLAFMRLLANYASQNITYH | |
| | | CKNSIAYMDEETGNLKKAVILQGSNDVELVAEGNSRFTYTV | |
| | | LVDGCSKKTNEWGKTIIEYKTNKPSRLPFLDIAPLDIGGAD | |
| | | QEFFVDIGPVCFK | |
| COL2A1 | P02458 | MIRLGAPQTLVLLTLLVAAVLRCQGQDVQEAGSCVQDGQRY | 3 |
| Collagen | | NDKDVWKPEPCRICVCDTGTVLCDDIICEDVKDCLSPEIPF | |
| alpha-1(II) | | GECCPICPTDLATASGQPGPKGQKGEPGDIKDIVGPKGPPG | |
| chain | | PQGPAGEQGPRGDRGDKGEKGAPGPRGRDGEPGTPGNPGPP | |
| | | GPPGPPGPPGLGGNFAAQMAGGFDEKAGGAQLGVMQGPMGP | |
| | | MGPRGPPGPAGAPGPQGFQGNPGEPGEPGVSGPMGPRGPPG | |
| | | PPGKPGDDGEAGKPGKAGERGPPGPQGARGFPGTPGLPGVK | |
| | | GHRGYPGLDGAKGEAGAPGVKGESGSPGENGSPGPMGPRGL | |
| | | PGERGRTGPAGAAGARGNDGQPGPAGPPGPVGPAGGPGFPG | |
| | | APGAKGEAGPTGARGPEGAQGPRGEPGTPGSPGPAGASGNP | |
| | | GTDGIPGAKGSAGAPGIAGAPGFPGPRGPPGPQGATGPLGP | |
| | | KGQTGEPGIAGFKGEQGPKGEPGPAGPQGAPGPAGEEGKRG | |
| | | ARGEPGGVGPIGPPGERGAPGNRGFPGQDGLAGPKGAPGER | |
| | | GPSGLAGPKGANGDPGRPGEPGLPGARGLTGRPGDAGPQGK | |
| | | VGPSGAPGEDGRPGPPGPQGARGQPGVMGFPGPKGANGEPG | |
| | | KAGEKGLPGAPGLRGLPGKDGETGAAGPPGPAGPAGERGEQ | |
| | | GAPGPSGFQGLPGPPGPPGEGGKPGDQGVPGEAGAPGLVGP | |
| | | RGERGFPGERGSPGAQGLQGPRGLPGTPGTDGPKGASGPAG | |
| | | PPGAQGPPGLQGMPGERGAAGIAGPKGDRGDVGEKGPEGAP | |
| | | GKDGGRGLTGPIGPPGPAGANGEKGEVGPPGPAGSAGARGA | |
| | | PGERGETGPPGPAGFAGPPGADGQPGAKGEQGEAGQKGDAG | |
| | | APGPQGPSGAPGPQGPTGVTGPKGARGAQGPPGATGFPGAA | |
| | | GRVGPPGSNGNPGPPGPPGPSGKDGPKGARGDSGPPGRAGE | |
| | | PGLQGPAGPPGEKGEPGDDGPSGAEGPPGPQGLAGQRGIVG | |
| | | LPGQRGERGFPGLPGPSGEPGKQGAPGASGDRGPPGPVGPP | |
| | | GLTGPAGEPGREGSPGADGPPGRDGAAGVKGDRGETGAVGA | |
| | | PGAPGPPGSPGPAGPTGKQGDRGEAGAQGPMGPSGPAGARG | |
| | | IQGPQGPRGDKGEAGEPGERGLKGHRGFTGLQGLPGPPGPS | |
| | | GDQGASGPAGPSGPRGPPGPVGPSGKDGANGIPGPIGPPGP | |
| | | RGRSGETGPAGPPGNPGPPGPPGPPGPGIDMSAFAGLGPRE | |
| | | KGPDPLQYMRADQAAGGLRQHDAEVDATLKSLNNQIESIRS | |
| | | PEGSRKNPARTCRDLKLCHPEWKSGDYWIDPNQGCTLDAMK | |
| | | VFCNMETGETCVYPNPANVPKKNWWSSKSKEKKHIWFGETI | |
| | | NGGFHFSYGDDNLAPNTANVQMTFLRLLSTEGSQNITYHCK | |
| | | NSIAYLDEAAGNLKKALLIQGSNDVEIRAEGNSRFTYTALK | |
| | | DGCTKHTGKWGKTVIEYRSQKTSRLPIIDIAPMDIGGPEQE | |
| | | FGVDIGPVCFL | |
| COL3A1 | P02461 | MMSFVQKGSWLLLALLHPTIILAQQEAVEGGCSHLGQSYAD | 4 |
| Collagen | | RDVWKPEPCQICVCDSGSVLCDDIICDDQELDCPNPEIPFG | |
| alpha-1(III) | | ECCAVCPQPPTAPTRPPNGQGPQGPKGDPGPPGIPGRNGDP | |
| chain | | GIPGQPGSPGSPGPPGICESCPTGPQNYSPQYDSYDVKSGV | |
| | | AVGGLAGYPGPAGPPGPPGPPGTSGHPGSPGSPGYQGPPGE | |
| | | PGQAGPSGPPGPPGAIGPSGPAGKDGESGRPGRPGERGLPG | |
| | | PPGIKGPAGIPGFPGMKGHRGFDGRNGEKGETGAPGLKGEN | |
| | | GLPGENGAPGPMGPRGAPGERGRPGLPGAAGARGNDGARGS | |
| | | DGQPGPPGPPGTAGFPGSPGAKGEVGPAGSPGSNGAPGQRG | |
| | | EPGPQGHAGAQGPPGPPGINGSPGGKGEMGPAGIPGAPGLM | |
| | | GARGPPGPAGANGAPGLRGGAGEPGKNGAKGEPGPRGERGE | |
| | | AGIPGVPGAKGEDGKDGSPGEPGANGLPGAAGERGAPGFRG | |
| | | PAGPNGIPGEKGPAGERGAPGPAGPRGAAGEPGRDGVPGGP | |
| | | GMRGMPGSPGGPGSDGKPGPPGSQGESGRPGPPGPSGPRGQ | |
| | | PGVMGFPGPKGNDGAPGKNGERGGPGGPGPQGPPGKNGETG | |
| | | PQGPPGPTGPGGDKGDTGPPGPQGLQGLPGTGGPPGENGKP | |
| | | GEPGPKGDAGAPGAPGGKGDAGAPGERGPPGLAGAPGLRGG | |
| | | AGPPGPEGGKGAAGPPGPPGAAGTPGLQGMPGERGGLGSPG | |
| | | PKGDKGEPGGPGADGVPGKDGPRGPTGPIGPPGPAGQPGDK | |
| | | GEGGAPGLPGIAGPRGSPGERGETGPPGPAGFPGAPGQNGE | |
| | | PGGKGERGAPGEKGEGGPPGVAGPPGGSGPAGPPGPQGVKG | |
| | | ERGSPGGPGAAGFPGARGLPGPPGSNGNPGPPGPSGSPGKD | |
| | | GPPGPAGNTGAPGSPGVSGPKGDAGQPGEKGSPGAQGPPGA | |
| | | PGPLGIAGITGARGLAGPPGMPGPRGSPGPQGVKGESGKPG | |
| | | ANGLSGERGPPGPQGLPGLAGTAGEPGRDGNPGSDGLPGRD | |
| | | GSPGGKGDRGENGSPGAPGAPGHPGPPGPVGPAGKSGDRGE | |
| | | SGPAGPAGAPGPAGSRGAPGPQGPRGDKGETGERGAAGIKG | |
| | | HRGFPGNPGAPGSPGPAGQQGAIGSPGPAGPRGPVGPSGPP | |
| | | GKDGTSGHPGPIGPPGPRGNRGERGSEGSPGHPGQPGPPGP | |
| | | PGAPGPCCGGVGAAAIAGIGGEKAGGFAPYYGDEPMDFKIN | |
| | | TDEIMTSLKSVNGQIESLISPDGSRKNPARNCRDLKFCHPE | |
| | | LKSGEYWVDPNQGCKLDAIKVFCNMETGETCISANPLNVPR | |
| | | KHWWTDSSAEKKHVWFGESMDGGFQFSYGNPELPEDVLDVH | |
| | | LAFLRLLSSRASQNITYHCKNSIAYMDQASGNVKKALKLMG | |
| | | SNEGEFKAEGNSKFTYTVLEDGCTKHTGEWSKTVFEYRTRK | |
| | | AVRLPIVDIAPYDIGGPDQEFGVDVGPVCFL | |
| COL4A1 | P02462 | MGPRLSVWLLLLPAALLLHEEHSRAAAKGGCAGSGCGKCDC | 5 |
| Collagen | | HGVKGQKGERGLPGLQGVIGFPGMQGPEGPQGPPGQKGDTG | |
| alpha-1(IV) | | EPGLPGTKGTRGPPGASGYPGNPGLPGIPGQDGPPGPPGIP | |
| chain | | GCNGTKGERGPLGPPGLPGFAGNPGPPGLPGMKGDPGEILG | |
| | | HVPGMLLKGERGFPGIPGTPGPPGLPGLQGPVGPPGFTGPP | |
| | | GPPGPPGPPGEKGQMGLSFQGPKGDKGDQGVSGPPGVPGQA | |
| | | QVQEKGDFATKGEKGQKGEPGFQGMPGVGEKGEPGKPGPRG | |
| | | KPGKDGDKGEKGSPGFPGEPGYPGLIGRQGPQGEKGEAGPP | |
| | | GPPGIVIGTGPLGEKGERGYPGTPGPRGEPGPKGFPGLPGQ | |
| | | PGPPGLPVPGQAGAPGFPGERGEKGDRGFPGTSLPGPSGRD | |
| | | GLPGPPGSPGPPGQPGYTNGIVECQPGPPGDQGPPGIPGQP | |
| | | GFIGEIGEKGQKGESCLICDIDGYRGPPGPQGPPGEIGFPG | |
| | | QPGAKGDRGLPGRDGVAGVPGPQGTPGLIGQPGAKGEPGEF | |
| | | YFDLRLKGDKGDPGFPGQPGMTGRAGSPGRDGHPGLPGPKG | |
| | | SPGSVGLKGERGPPGGVGFPGSRGDTGPPGPPGYGPAGPIG | |
| | | DKGQAGFPGGPGSPGLPGPKGEPGKIVPLPGPPGAEGLPGS | |
| | | PGFPGPQGDRGFPGTPGRPGLPGEKGAVGQPGIGFPGPPGP | |
| | | KGVDGLPGDMGPPGTPGRPGFNGLPGNPGVQGQKGEPGVGL | |
| | | PGLKGLPGLPGIPGTPGEKGSIGVPGVPGEHGAIGPPGLQG | |
| | | IRGEPGPPGLPGSVGSPGVPGIGPPGARGPPGGQGPPGLSG | |
| | | PPGIKGEKGFPGFPGLDMPGPKGDKGAQGLPGITGQSGLPG | |
| | | LPGQQGAPGIPGFPGSKGEMGVMGTPGQPGSPGPVGAPGLP | |
| | | GEKGDHGFPGSSGPRGDPGLKGDKGDVGLPGKPGSMDKVDM | |
| | | GSMKGQKGDQGEKGQIGPIGEKGSRGDPGTPGVPGKDGQAG | |
| | | QPGQPGPKGDPGISGTPGAPGLPGPKGSVGGMGLPGTPGEK | |
| | | GVPGIPGPQGSPGLPGDKGAKGEKGQAGPPGIGIPGLRGEK | |
| | | GDQGIAGFPGSPGEKGEKGSIGIPGMPGSPGLKGSPGSVGY | |
| | | PGSPGLPGEKGDKGLPGLDGIPGVKGEAGLPGTPGPTGPAG | |
| | | QKGEPGSDGIPGSAGEKGEPGLPGRGFPGFPGAKGDKGSKG | |
| | | EVGFPGLAGSPGIPGSKGEQGFMGPPGPQGQPGLPGSPGHA | |
| | | TEGPKGDRGPQGQPGLPGLPGPMGPPGLPGIDGVKGDKGNP | |
| | | GWPGAPGVPGPKGDPGFQGMPGIGGSPGITGSKGDMGPPGV | |
| | | PGFQGPKGLPGLQGIKGDQGDQGVPGAKGLPGPPGPPGPYD | |
| | | IIKGEPGLPGPEGPPGLKGLQGLPGPKGQQGVTGLVGIPGP | |
| | | PGIPGFDGAPGQKGEMGPAGPTGPRGFPGPPGPDGLPGSMG | |
| | | PPGTPSVDHGFLVTRHSQTIDDPQCPSGTKILYHGYSLLYV | |
| | | QGNERAHGQDLGTAGSCLRKFSTMPFLFCNINNVCNFASRN | |
| | | DYSYWLSTPEPMPMSMAPITGENIRPFISRCAVCEAPAMVM | |
| | | AVHSQTIQIPPCPSGWSSLWIGYSFVMHTSAGAEGSGQALA | |
| | | SPGSCLEEFRSAPFIECHGRGTCNYYANAYSFWLATIERSE | |
| | | MFKKPTPSTLKAGELRTHVSRCQVCMRRT | |
| COL4A2 | P08572 | MGRDQRAVAGPALRRWLLLGTVTVGFLAQSVLAGVKKFDVP | 6 |
| Collagen | | CGGRDCSGGCQCYPEKGGRGQPGPVGPQGYNGPPGLQGFPG | |
| alpha-2(IV) | | LQGRKGDKGERGAPGVTGPKGDVGARGVSGFPGADGIPGHP | |
| chain | | GQGGPRGRPGYDGCNGTQGDSGPQGPPGSEGFTGPPGPQGP | |
| | | KGQKGEPYALPKEERDRYRGEPGEPGLVGFQGPPGRPGHVG | |
| | | QMGPVGAPGRPGPPGPPGPKGQQGNRGLGFYGVKGEKGDVG | |
| | | QPGPNGIPSDTLHPIIAPTGVTFHPDQYKGEKGSEGEPGIR | |
| | | GISLKGEEGIMGFPGLRGYPGLSGEKGSPGQKGSRGLDGYQ | |
| | | GPDGPRGPKGEAGDPGPPGLPAYSPHPSLAKGARGDPGFPG | |
| | | AQGEPGSQGEPGDPGLPGPPGLSIGDGDQRRGLPGEMGPKG | |
| | | FIGDPGIPALYGGPPGPDGKRGPPGPPGLPGPPGPDGFLFG | |
| | | LKGAKGRAGFPGLPGSPGARGPKGWKGDAGECRCTEGDEAI | |
| | | KGLPGLPGPKGFAGINGEPGRKGDRGDPGQHGLPGFPGLKG | |
| | | VPGNIGAPGPKGAKGDSRTITTKGERGQPGVPGVPGMKGDD | |
| | | GSPGRDGLDGFPGLPGPPGDGIKGPPGDPGYPGIPGTKGTP | |
| | | GEMGPPGLGLPGLKGQRGFPGDAGLPGPPGFLGPPGPAGTP | |
| | | GQIDCDTDVKRAVGGDRQEAIQPGCIGGPKGLPGLPGPPGP | |
| | | TGAKGLRGIPGFAGADGGPGPRGLPGDAGREGFPGPPGFIG | |
| | | PRGSKGAVGLPGPDGSPGPIGLPGPDGPPGERGLPGEVLGA | |
| | | QPGPRGDAGVPGQPGLKGLPGDRGPPGFRGSQGMPGMPGLK | |
| | | GQPGLPGPSGQPGLYGPPGLHGFPGAPGQEGPLGLPGIPGR | |
| | | EGLPGDRGDPGDTGAPGPVGMKGLSGDRGDAGFTGEQGHPG | |
| | | SPGFKGIDGMPGTPGLKGDRGSPGMDGFQGMPGLKGRPGFP | |
| | | GSKGEAGFFGIPGLKGLAGEPGFKGSRGDPGPPGPPPVILP | |
| | | GMKDIKGEKGDEGPMGLKGYLGAKGIQGMPGIPGLSGIPGL | |
| | | PGRPGHIKGVKGDIGVPGIPGLPGFPGVAGPPGITGFPGFI | |
| | | GSRGDKGAPGRAGLYGEIGATGDFGDIGDTINLPGRPGLKG | |
| | | ERGTTGIPGLKGFFGEKGTEGDIGFPGITGVTGVQGPPGLK | |
| | | GQTGFPGLTGPPGSQGELGRIGLPGGKGDDGWPGAPGLPGF | |
| | | PGLRGIRGLHGLPGTKGFPGSPGSDIHGDPGFPGPPGERGD | |
| | | PGEANTLPGPVGVPGQKGDQGAPGERGPPGSPGLQGFPGIT | |
| | | PPSNISGAPGDKGAPGIFGLKGYRGPPGPPGSAALPGSKGD | |
| | | TGNPGAPGTPGTKGWAGDSGPQGRPGVFGLPGEKGPRGEQG | |
| | | FMGNTGPTGAVGDRGPKGPKGDPGFPGAPGTVGAPGIAGIP | |
| | | QKIAVQPGTVGPQGRRGPPGAPGEMGPQGPPGEPGFRGAPG | |
| | | KAGPQGRGGVSAVPGFRGDEGPIGHQGPIGQEGAPGRPGSP | |
| | | GLPGMPGRSVSIGYLLVKHSQTDQEPMCPVGMNKLWSGYSL | |
| | | LYFEGQEKAHNQDLGLAGSCLARFSTMPFLYCNPGDVCYYA | |
| | | SRNDKSYWLSTTAPLPMMPVAEDEIKPYISRCSVCEAPAIA | |
| | | IAVHSQDVSIPHCPAGWRSLWIGYSFLMHTAAGDEGGGQSL | |
| | | VSPGSCLEDFRATPFIECNGGRGTCHYYANKYSFWLTTIPE | |
| | | QSFQGSPSADTLKAGLIRTHISRCQVCMKNL | |
| COL4A3 | Q01955 | MSARTAPRPQVLLLPLLLVLLAAAPAASKGCVCKDKGQCFC | 7 |
| Collagen | | DGAKGEKGEKGFPGPPGSPGQKGFTGPEGLPGPQGPKGFPG | |
| alpha-3(IV) | | LPGLTGSKGVRGISGLPGFSGSPGLPGTPGNTGPYGLVGVP | |
| chain | | GCSGSKGEQGFPGLPGTLGYPGIPGAAGLKGQKGAPAKEED | |
| | | IELDAKGDPGLPGAPGPQGLPGPPGFPGPVGPPGPPGFFGF | |
| | | PGAMGPRGPKGHMGERVIGHKGERGVKGLTGPPGPPGTVIV | |
| | | TLTGPDNRTDLKGEKGDKGAMGEPGPPGPSGLPGESYGSEK | |
| | | GAPGDPGLQGKPGKDGVPGFPGSEGVKGNRGFPGLMGEDGI | |
| | | KGQKGDIGPPGFRGPTEYYDTYQEKGDEGTPGPPGPRGARG | |
| | | PQGPSGPPGVPGSPGSSRPGLRGAPGWPGLKGSKGERGRPG | |
| | | KDAMGTPGSPGCAGSPGLPGSPGPPGPPGDIVFRKGPPGDH | |
| | | GLPGYLGSPGIPGVDGPKGEPGLLCTQCPYIPGPPGLPGLP | |
| | | GLHGVKGIPGRQGAAGLKGSPGSPGNTGLPGFPGFPGAQGD | |
| | | PGLKGEKGETLQPEGQVGVPGDPGLRGQPGRKGLDGIPGTP | |
| | | GVKGLPGPKGELALSGEKGDQGPPGDPGSPGSPGPAGPAGP | |
| | | PGYGPQGEPGLQGTQGVPGAPGPPGEAGPRGELSVSIPVPG | |
| | | PPGPPGPPGHPGPQGPPGIPGSLGKCGDPGLPGPDGEPGIP | |
| | | GIGFPGPPGPKGDQGFPGTKGSLGCPGKMGEPGLPGKPGLP | |
| | | GAKGEPAVAMPGGPGTPGFPGERGNSGEHGEIGLPGLPGLP | |
| | | GTPGNEGLDGPRGDPGQPGPPGEQGPPGRCIEGPRGAQGLP | |
| | | GLNGLKGQQGRRGKTGPKGDPGIPGLDRSGFPGETGSPGIP | |
| | | GHQGEMGPLGQRGYPGNPGILGPPGEDGVIGMMGFPGAIGP | |
| | | PGPPGNPGTPGQRGSPGIPGVKGQRGTPGAKGEQGDKGNPG | |
| | | PSEISHVIGDKGEPGLKGFAGNPGEKGNRGVPGMPGLKGLK | |
| | | GLPGPAGPPGPRGDLGSTGNPGEPGLRGIPGSMGNMGMPGS | |
| | | KGKRGTLGFPGRAGRPGLPGIHGLQGDKGEPGYSEGTRPGP | |
| | | PGPTGDPGLPGDMGKKGEMGQPGPPGHLGPAGPEGAPGSPG | |
| | | SPGLPGKPGPHGDLGFKGIKGLLGPPGIRGPPGLPGFPGSP | |
| | | GPMGIRGDQGRDGIPGPAGEKGETGLLRAPPGPRGNPGAQG | |
| | | AKGDRGAPGFPGLPGRKGAMGDAGPRGPTGIEGFPGPPGLP | |
| | | GAIIPGQTGNRGPPGSRGSPGAPGPPGPPGSHVIGIKGDKG | |
| | | SMGHPGPKGPPGTAGDMGPPGRLGAPGTPGLPGPRGDPGFQ | |
| | | GFPGVKGEKGNPGFLGSIGPPGPIGPKGPPGVRGDPGTLKI | |
| | | ISLPGSPGPPGTPGEPGMQGEPGPPGPPGNLGPCGPRGKPG | |
| | | KDGKPGTPGPAGEKGNKGSKGEPGPAGSDGLPGLKGKRGDS | |
| | | GSPATWTTRGFVFTRHSQTTAIPSCPEGTVPLYSGFSFLFV | |
| | | QGNQRAHGQDLGTLGSCLQRFTTMPFLFCNVNDVCNFASRN | |
| | | DYSYWLSTPALMPMNMAPITGRALEPYISRCTVCEGPAIAI | |
| | | AVHSQTTDIPPCPHGWISLWKGFSFIMFTSAGSEGTGQALA | |
| | | SPGSCLEEFRASPFLECHGRGTCNYYSNSYSFWLASLNPER | |
| | | MFRKPIPSTVKAGELEKIISRCQVCMKKRH | |
| COL4A4 | P54320 | MWSLHIVLMRCSFRLTKSLATGPWSLILILFSVQYVYGSGK | 8 |
| Collagen | | KYIGPCGGRDCSVCHCVPEKGSRGPPGPPGPQGPIGPLGAP | |
| alpha-4(IV) | | GPIGLSGEKGMRGDRGPPGAAGDKGDKGPTGVPGFPGLDGI | |
| chain | | PGHPGPPGPRGKPGMSGHNGSRGDPGFPGGRGALGPGGPLG | |
| | | HPGEKGEKGNSVFILGAVKGIQGDRGDPGLPGLPGSWGAGG | |
| | | PAGPTGYPGEPGLVGPPGQPGRPGLKGNPGVGVKGQMGDPG | |
| | | EVGQQGSPGPTLLVEPPDFCLYKGEKGIKGIPGMVGLPGPP | |
| | | GRKGESGIGAKGEKGIPGFPGPRGDPGSYGSPGFPGLKGEL | |
| | | GLVGDPGLFGLIGPKGDPGNRGHPGPPGVLVTPPLPLKGPP | |
| | | GDPGFPGRYGETGDVGPPGPPGLLGRPGEACAGMIGPPGPQ | |
| | | GFPGLPGLPGEAGIPGRPDSAPGKPGKPGSPGLPGAPGLQG | |
| | | LPGSSVIYCSVGNPGPQGIKGKVGPPGGRGPKGEKGNEGLC | |
| | | ACEPGPMGPPGPPGLPGRQGSKGDLGLPGWLGTKGDPGPPG | |
| | | AEGPPGLPGKHGASGPPGNKGAKGDMVVSRVKGHKGERGPD | |
| | | GPPGFPGQPGSHGRDGHAGEKGDPGPPGDHEDATPGGKGFP | |
| | | GPLGPPGKAGPVGPPGLGFPGPPGERGHPGVPGHPGVRGPD | |
| | | GLKGQKGDTISCNVTYPGRHGPPGFDGPPGPKGFPGPQGAP | |
| | | GLSGSDGHKGRPGTPGTAEIPGPPGFRGDMGDPGFGGEKGS | |
| | | SPVGPPGPPGSPGVNGQKGIPGDPAFGHLGPPGKRGLSGVP | |
| | | GIKGPRGDPGCPGAEGPAGIPGFLGLKGPKGREGHAGFPGV | |
| | | PGPPGHSCERGAPGIPGQPGLPGYPGSPGAPGGKGQPGDVG | |
| | | PPGPAGMKGLPGLPGRPGAHGPPGLPGIPGPFGDDGLPGPP | |
| | | GPKGPRGLPGFPGFPGERGKPGAEGCPGAKGEPGEKGMSGL | |
| | | PGDRGLRGAKGAIGPPGDEGEMAIISQKGTPGEPGPPGDDG | |
| | | FPGERGDKGTPGMQGRRGEPGRYGPPGFHRGEPGEKGQPGP | |
| | | PGPPGPPGSTGLRGFIGFPGLPGDQGEPGSPGPPGFSGIDG | |
| | | ARGPKGNKGDPASHFGPPGPKGEPGSPGCPGHFGASGEQGL | |
| | | PGIQGPRGSPGRPGPPGSSGPPGCPGDHGMPGLRGQPGEMG | |
| | | DPGPRGLQGDPGIPGPPGIKGPSGSPGLNGLHGLKGQKGTK | |
| | | GASGLHDVGPPGPVGIPGLKGERGDPGSPGISPPGPRGKKG | |
| | | PPGPPGSSGPPGPAGATGRAPKDIPDPGPPGDQGPPGPDGP | |
| | | RGAPGPPGLPGSVDLLRGEPGDCGLPGPPGPPGPPGPPGYK | |
| | | GFPGCDGKDGQKGPVGFPGPQGPHGFPGPPGEKGLPGPPGR | |
| | | KGPTGLPGPRGEPGPPADVDDCPRIPGLPGAPGMRGPEGAM | |
| | | GLPGMRGPSGPGCKGEPGLDGRRGVDGVPGSPGPPGRKGDT | |
| | | GEDGYPGGPGPPGPIGDPGPKGFGPGYLGGFLLVLHSQTDQ | |
| | | EPTCPLGMPRLWTGYSLLYLEGQEKAHNQDLGLAGSCLPVF | |
| | | STLPFAYCNIHQVCHYAQRNDRSYWLASAAPLPMMPLSEEA | |
| | | IRPYVSRCAVCEAPAQAVAVHSQDQSIPPCPQTWRSLWIGY | |
| | | SFLMHTGAGDQGGGQALMSPGSCLEDFRAAPFLECQGRQGT | |
| | | CHFFANKYSFWLTTVKADLQFSSAPAPDTLKESQAQRQKIS | |
| | | RCQVCVKYS | |
| COL4A5 | P29400 | MKLRGVSLAAGLFLLALSLWGQPAEAAACYGCSPGSKCDCS | 9 |
| Collagen | | GIKGEKGERGFPGLEGHPGLPGFPGPEGPPGPRGQKGDDGI | |
| alpha-5(IV) | | PGPPGPKGIRGPPGLPGFPGTPGLPGMPGHDGAPGPQGIPG | |
| chain | | CNGTKGERGFPGSPGFPGLQGPPGPPGIPGMKGEPGSIIMS | |
| | | SLPGPKGNPGYPGPPGIQGLPGPTGIPGPIGPPGPPGLMGP | |
| | | PGPPGLPGPKGNMGLNFQGPKGEKGEQGLQGPPGPPGQISE | |
| | | QKRPIDVEFQKGDQGLPGDRGPPGPPGIRGPPGPPGGEKGE | |
| | | KGEQGEPGKRGKPGKDGENGQPGIPGLPGDPGYPGEPGRDG | |
| | | EKGQKGDTGPPGPPGLVIPRPGTGITIGEKGNIGLPGLPGE | |
| | | KGERGFPGIQGPPGLPGPPGAAVMGPPGPPGFPGERGQKGD | |
| | | EGPPGISIPGPPGLDGQPGAPGLPGPPGPAGPHIPPSDEIC | |
| | | EPGPPGPPGSPGDKGLQGEQGVKGDKGDTCFNCIGTGISGP | |
| | | PGQPGLPGLPGPPGSLGFPGQKGEKGQAGATGPKGLPGIPG | |
| | | APGAPGFPGSKGEPGDILTFPGMKGDKGELGSPGAPGLPGL | |
| | | PGTPGQDGLPGLPGPKGEPGGITFKGERGPPGNPGLPGLPG | |
| | | NIGPMGPPGFGPPGPVGEKGIQGVAGNPGQPGIPGPKGDPG | |
| | | QTITQPGKPGLPGNPGRDGDVGLPGDPGLPGQPGLPGIPGS | |
| | | KGEPGIPGIGLPGPPGPKGFPGIPGPPGAPGTPGRIGLEGP | |
| | | PGPPGFPGPKGEPGFALPGPPGPPGLPGFKGALGPKGDRGF | |
| | | PGPPGPPGRTGLDGLPGPKGDVGPNGQPGPMGPPGLPGIGV | |
| | | QGPPGPPGIPGPIGQPGLHGIPGEKGDPGPPGLDVPGPPGE | |
| | | RGSPGIPGAPGPIGPPGSPGLPGKAGASGFPGTKGEMGMMG | |
| | | PPGPPGPLGIPGRSGVPGLKGDDGLQGQPGLPGPTGEKGSK | |
| | | GEPGLPGPPGPMDPNLLGSKGEKGEPGLPGIPGVSGPKGYQ | |
| | | GLPGDPGQPGLSGQPGLPGPPGPKGNPGLPGQPGLIGPPGL | |
| | | KGTIGDMGFPGPQGVEGPPGPSGVPGQPGSPGLPGQKGDKG | |
| | | DPGISSIGLPGLPGPKGEPGLPGYPGNPGIKGSVGDPGLPG | |
| | | LPGTPGAKGQPGLPGFPGTPGPPGPKGISGPPGNPGLPGEP | |
| | | GPVGGGGHPGQPGPPGEKGKPGQDGIPGPAGQKGEPGQPGF | |
| | | GNPGPPGLPGLSGQKGDGGLPGIPGNPGLPGPKGEPGFHGF | |
| | | PGVQGPPGPPGSPGPALEGPKGNPGPQGPPGRPGLPGPEGP | |
| | | PGLPGNGGIKGEKGNPGQPGLPGLPGLKGDQGPPGLQGNPG | |
| | | RPGLNGMKGDPGLPGVPGFPGMKGPSGVPGSAGPEGEPGLI | |
| | | GPPGPPGLPGPSGQSIIIKGDAGPPGIPGQPGLKGLPGPQG | |
| | | PQGLPGPTGPPGDPGRNGLPGFDGAGGRKGDPGLPGQPGTR | |
| | | GLDGPPGPDGLQGPPGPPGTSSVAHGFLITRHSQTTDAPQC | |
| | | PQGTLQVYEGFSLLYVQGNKRAHGQDLGTAGSCLRRFSTMP | |
| | | FMFCNINNVCNFASRNDYSYWLSTPEPMPMSMQPLKGQSIQ | |
| | | PFISRCAVCEAPAVVIAVHSQTIQIPHCPQGWDSLWIGYSF | |
| | | MMHTSAGAEGSGQALASPGSCLEEFRSAPFIECHGRGTCNY | |
| | | YANSYSFWLATVDVSDMFSKPQSETLKAGDLRTRISRCQVC | |
| | | MKRT | |
| COL4A6 | Q14031 | MLINKLWLLLVTLCLTEELAAAGEKSYGKPCGGQDCSGSCQ | 10 |
| Collagen | | CFPEKGARGRPGPIGIQGPTGPQGFTGSTGLSGLKGERGFP | |
| alpha-6(IV) | | GLLGPYGPKGDKGPMGVPGFLGINGIPGHPGQPGPRGPPGL | |
| chain | | DGCNGTQGAVGFPGPDGYPGLLGPPGLPGQKGSKGDPVLAP | |
| | | GSFKGMKGDPGLPGLDGITGPQGAPGFPGAVGPAGPPGLQG | |
| | | PPGPPGPLGPDGNMGLGFQGEKGVKGDVGLPGPAGPPPSIG | |
| | | ELEFMGFPKGKKGSKGEPGPKGFPGISGPPGFPGLGTTGEK | |
| | | GEKGEKGIPGLPGPRGPMGSEGVQGPPGQQGKKGTLGFPGL | |
| | | NGFQGIEGQKGDIGLPGPDVFIDIDGAVISGNPGDPGVPGL | |
| | | PGLKGDEGIQGLRGPSGVPGLPALSGVPGALGPQGFPGLKG | |
| | | DQGNPGRTTIGAAGLPGRDGLPGPPGPPGPPSPEFETETLH | |
| | | NKESGFPGLRGEQGPKGNLGLKGIKGDSGFCACDGGVPNTG | |
| | | PPGEPGPPGPWGLIGLPGLKGARGDRGSGGAQGPAGAPGLV | |
| | | GPLGPSGPKGKKGEPILSTIQGMPGDRGDSGSQGFRGVIGE | |
| | | PGKDGVPGLPGLPGLPGDGGQGFPGEKGLPGLPGEKGHPGP | |
| | | PGLPGNGLPGLPGPRGLPGDKGKDGLPGQQGLPGSKGITLP | |
| | | CIIPGSYGPSGFPGTPGFPGPKGSRGLPGTPGQPGSSGSKG | |
| | | EPGSPGLVHLPELPGFPGPRGEKGLPGFPGLPGKDGLPGMI | |
| | | GSPGLPGSKGATGDIFGAENGAPGEQGLQGLTGHKGFLGDS | |
| | | GLPGLKGVHGKPGLLGPKGERGSPGTPGQVGQPGTPGSSGP | |
| | | YGIKGKSGLPGAPGFPGISGHPGKKGTRGKKGPPGSIVKKG | |
| | | LPGLKGLPGNPGLVGLKGSPGSPGVAGLPALSGPKGEKGSV | |
| | | GFVGFPGIPGLPGIPGTRGLKGIPGSTGKMGPSGRAGTPGE | |
| | | KGDRGNPGPVGIPSPRRPMSNLWLKGDKGSQGSAGSNGFPG | |
| | | PRGDKGEAGRPGPPGLPGAPGLPGIIKGVSGKPGPPGFMGI | |
| | | RGLPGLKGSSGITGFPGMPGESGSQGIRGSPGLPGASGLPG | |
| | | LKGDNGQTVEISGSPGPKGQPGESGFKGTKGRDGLIGNIGF | |
| | | PGNKGEDGKVGVSGDVGLPGAPGFPGVAGMRGEPGLPGSSG | |
| | | HQGAIGPLGSPGLIGPKGFPGFPGLHGLNGLPGTKGTHGTP | |
| | | GPSITGVPGPAGLPGPKGEKGYPGIGIGAPGKPGLRGQKGD | |
| | | RGFPGLQGPAGLPGAPGISLPSLIAGQPGDPGRPGLDGERG | |
| | | RPGPAGPPGPPGPSSNQGDTGDPGFPGIPGPKGPKGDQGIP | |
| | | GFSGLPGELGLKGMRGEPGFMGTPGKVGPPGDPGFPGMKGK | |
| | | AGPRGSSGLQGDPGQTPTAEAVQVPPGPLGLPGIDGIPGLT | |
| | | GDPGAQGPVGLQGSKGLPGIPGKDGPSGLPGPPGALGDPGL | |
| | | PGLQGPPGFEGAPGQQGPFGMPGMPGQSMRVGYTLVKHSQS | |
| | | EQVPPCPIGMSQLWVGYSLLFVEGQEKAHNQDLGFAGSCLP | |
| | | RFSTMPFIYCNINEVCHYARRNDKSYWLSTTAPIPMMPVSQ | |
| | | TQIPQYISRCSVCEAPSQAIAVHSQDITIPQCPLGWRSLWI | |
| | | GYSFLMHTAAGAEGGGQSLVSPGSCLEDFRATPFIECSGAR | |
| | | GTCHYFANKYSFWLTTVEERQQFGELPVSETLKAGQLHTRV | |
| | | SRCQVCMKSL | |
| COL5A1 | P20908 | MDVHTRWKARSALRPGAPLLPPLLLLLLWAPPPSRAAQPAD | 11 |
| Collagen | | LLKVLDFHNLPDGITKTTGFCATRRSSKGPDVAYRVTKDAQ | |
| alpha-1(V) | | LSAPTKQLYPASAFPEDFSILTTVKAKKGSQAFLVSIYNEQ | |
| chain | | GIQQIGLELGRSPVFLYEDHTGKPGPEDYPLFRGINLSDGK | |
| | | WHRIALSVHKKNVTLILDCKKKTTKFLDRSDHPMIDINGII | |
| | | VFGTRILDEEVFEGDIQQLLFVSDHRAAYDYCEHYSPDCDT | |
| | | AVPDTPQSQDPNPDEYYTEGDGEGETYYYEYPYYEDPEDLG | |
| | | KEPTPSKKPVEAAKETTEVPEELTPTPTEAAPMPETSEGAG | |
| | | KEEDVGIGDYDYVPSEDYYTPSPYDDLTYGEGEENPDQPTD | |
| | | PGAGAEIPTSTADTSNSSNPAPPPGEGADDLEGEFTEETIR | |
| | | NLDENYYDPYYDPTSSPSEIGPGMPANQDTIYEGIGGPRGE | |
| | | KGQKGEPAIIEPGMLIEGPPGPEGPAGLPGPPGTMGPTGQV | |
| | | GDPGERGPPGRPGLPGADGLPGPPGTMLMLPFRFGGGGDAG | |
| | | SKGPMVSAQESQAQAILQQARLALRGPAGPMGLTGRPGPVG | |
| | | PPGSGGLKGEPGDVGPQGPRGVQGPPGPAGKPGRRGRAGSD | |
| | | GARGMPGQTGPKGDRGFDGLAGLPGEKGHRGDPGPSGPPGP | |
| | | PGDDGERGDDGEVGPRGLPGEPGPRGLLGPKGPPGPPGPPG | |
| | | VTGMDGQPGPKGNVGPQGEPGPPGQQGNPGAQGLPGPQGAI | |
| | | GPPGEKGPLGKPGLPGMPGADGPPGHPGKEGPPGEKGGQGP | |
| | | PGPQGPIGYPGPRGVKGADGIRGLKGTKGEKGEDGFPGFKG | |
| | | DMGIKGDRGEIGPPGPRGEDGPEGPKGRGGPNGDPGPLGPP | |
| | | GEKGKLGVPGLPGYPGRQGPKGSIGFPGFPGANGEKGGRGT | |
| | | PGKPGPRGQRGPTGPRGERGPRGITGKPGPKGNSGGDGPAG | |
| | | PPGERGPNGPQGPTGFPGPKGPPGPPGKDGLPGHPGQRGET | |
| | | GFQGKTGPPGPPGVVGPQGPTGETGPMGERGHPGPPGPPGE | |
| | | QGLPGLAGKEGTKGDPGPAGLPGKDGPPGLRGFPGDRGLPG | |
| | | PVGALGLKGNEGPPGPPGPAGSPGERGPAGAAGPIGIPGRP | |
| | | GPQGPPGPAGEKGAPGEKGPQGPAGRDGLQGPVGLPGPAGP | |
| | | VGPPGEDGDKGEIGEPGQKGSKGDKGEQGPPGPTGPQGPIG | |
| | | QPGPSGADGEPGPRGQQGLFGQKGDEGPRGFPGPPGPVGLQ | |
| | | GLPGPPGEKGETGDVGQMGPPGPPGPRGPSGAPGADGPQGP | |
| | | PGGIGNPGAVGEKGEPGEAGEPGLPGEGGPPGPKGERGEKG | |
| | | ESGPSGAAGPPGPKGPPGDDGPKGSPGPVGFPGDPGPPGEP | |
| | | GPAGQDGPPGDKGDDGEPGQTGSPGPTGEPGPSGPPGKRGP | |
| | | PGPAGPEGRQGEKGAKGEAGLEGPPGKTGPIGPQGAPGKPG | |
| | | PDGLRGIPGPVGEQGLPGSPGPDGPPGPMGPPGLPGLKGDS | |
| | | GPKGEKGHPGLIGLIGPPGEQGEKGDRGLPGPQGSSGPKGE | |
| | | QGITGPSGPIGPPGPPGLPGPPGPKGAKGSSGPTGPKGEAG | |
| | | HPGPPGPPGPPGEVIQPLPIQASRTRRNIDASQLLDDGNGE | |
| | | NYVDYADGMEEIFGSLNSLKLEIEQMKRPLGTQQNPARTCK | |
| | | DLQLCHPDFPDGEYWVDPNQGCSRDSFKVYCNFTAGGSTCV | |
| | | FPDKKSEGARITSWPKENPGSWFSEFKRGKLLSYVDAEGNP | |
| | | VGVVQMTFLRLLSASAHQNVTYHCYQSVAWQDAATGSYDKA | |
| | | LRFLGSNDEEMSYDNNPYIRALVDGCATKKGYQKTVLEIDI | |
| | | PKVEQVPIVDIMFNDFGEASQKFGFEVGPACFMG | |
| COL5A2 | P05997 | MMANWAEARPLLILIVLLGQFVSIKAQEEDEDEGYGEEIAC | 12 |
| Collagen | | TQNGQMYLNRDIWKPAPCQICVCDNGAILCDKIECQDVLDC | |
| alpha-2(V) | | ADPVTPPGECCPVCSQTPGGGNTNFGRGRKGQKGEPGLVPV | |
| chain | | VTGIRGRPGPAGPPGSQGPRGERGPKGRPGPRGPQGIDGEP | |
| | | GVPGQPGAPGPPGHPSHPGPDGLSRPFSAQMAGLDEKSGLG | |
| | | SQVGLMPGSVGPVGPRGPQGLQGQQGGAGPTGPPGEPGDPG | |
| | | PMGPIGSRGPEGPPGKPGEDGEPGRNGNPGEVGFAGSPGAR | |
| | | GFPGAPGLPGLKGHRGHKGLEGPKGEVGAPGSKGEAGPTGP | |
| | | MGAMGPLGPRGMPGERGRLGPQGAPGQRGAHGMPGKPGPMG | |
| | | PLGIPGSSGFPGNPGMKGEAGPTGARGPEGPQGQRGETGPP | |
| | | GPVGSPGLPGAIGTDGTPGAKGPTGSPGTSGPPGSAGPPGS | |
| | | PGPQGSTGPQGIRGQPGDPGVPGFKGEAGPKGEPGPHGIQG | |
| | | PIGPPGEEGKRGPRGDPGTVGPPGPVGERGAPGNRGFPGSD | |
| | | GLPGPKGAQGERGPVGSSGPKGSQGDPGRPGEPGLPGARGL | |
| | | TGNPGVQGPEGKLGPLGAPGEDGRPGPPGSIGIRGQPGSMG | |
| | | LPGPKGSSGDPGKPGEAGNAGVPGQRGAPGKDGEVGPSGPV | |
| | | GPPGLAGERGEQGPPGPTGFQGLPGPPGPPGEGGKPGDQGV | |
| | | PGDPGAVGPLGPRGERGNPGERGEPGITGLPGEKGMAGGHG | |
| | | PDGPKGSPGPSGTPGDTGPPGLQGMPGERGIAGTPGPKGDR | |
| | | GGIGEKGAEGTAGNDGARGLPGPLGPPGPAGPTGEKGEPGP | |
| | | RGLVGPPGSRGNPGSRGENGPTGAVGFAGPQGPDGQPGVKG | |
| | | EPGEPGQKGDAGSPGPQGLAGSPGPHGPNGVPGLKGGRGTQ | |
| | | GPPGATGFPGSAGRVGPPGPAGAPGPAGPLGEPGKEGPPGL | |
| | | RGDPGSHGRVGDRGPAGPPGGPGDKGDPGEDGQPGPDGPPG | |
| | | PAGTTGQRGIVGMPGQRGERGMPGLPGPAGTPGKVGPTGAT | |
| | | GDKGPPGPVGPPGSNGPVGEPGPEGPAGNDGTPGRDGAVGE | |
| | | RGDRGDPGPAGLPGSQGAPGTPGPVGAPGDAGQRGDPGSRG | |
| | | PIGPPGRAGKRGLPGPQGPRGDKGDHGDRGDRGQKGHRGFT | |
| | | GLQGLPGPPGPNGEQGSAGIPGPFGPRGPPGPVGPSGKEGN | |
| | | PGPLGPIGPPGVRGSVGEAGPEGPPGEPGPPGPPGPPGHLT | |
| | | AALGDIMGHYDESMPDPLPEFTEDQAAPDDKNKTDPGVHAT | |
| | | LKSLSSQIETMRSPDGSKKHPARTCDDLKLCHSAKQSGEYW | |
| | | IDPNQGSVEDAIKVYCNMETGETCISANPSSVPRKTWWASK | |
| | | SPDNKPVWYGLDMNRGSQFAYGDHQSPNTAITQMTFLRLLS | |
| | | KEASQNITYICKNSVGYMDDQAKNLKKAVVLKGANDLDIKA | |
| | | EGNIRFRYIVLQDTCSKRNGNVGKTVFEYRTQNVARLPIID | |
| | | LAPVDVGGTDQEFGVEIGPVCFV | |
| COL5A3 | P25940 | MGNRRDLGQPRAGLCLLLAALQLLPGTQADPVDVLKALGVQ | 13 |
| Collagen | | GGQAGVPEGPGFCPQRTPEGDRAFRIGQASTLGIPTWELFP | |
| alpha-3(V) | | EGHFPENFSLLITLRGQPANQSVLLSIYDERGARQLGLALG | |
| chain | | PALGLLGDPFRPLPQQVNLTDGRWHRVAVSIDGEMVTLVAD | |
| | | CEAQPPVLGHGPRFISIAGLTVLGTQDLGEKTFEGDIQELL | |
| | | ISPDPQAAFQACERYLPDCDNLAPAATVAPQGEPETPRPRR | |
| | | KGKGKGRKKGRGRKGKGRKKNKEIWTSSPPPDSAENQTSTD | |
| | | IPKTETPAPNLPPTPTPLVVTSTVTTGLNATILERSLDPDS | |
| | | GTELGTLETKAAREDEEGDDSTMGPDFRAAEYPSRTQFQIF | |
| | | PGAGEKGAKGEPAVIEKGQQFEGPPGAPGPQGVVGPSGPPG | |
| | | PPGFPGDPGPPGPAGLPGIPGIDGIRGPPGTVIMMPFQFAG | |
| | | GSFKGPPVSFQQAQAQAVLQQTQLSMKGPPGPVGLTGRPGP | |
| | | VGLPGHPGLKGEEGAEGPQGPRGLQGPHGPPGRVGKMGRPG | |
| | | ADGARGLPGDTGPKGDRGFDGLPGLPGEKGQRGDFGHVGQP | |
| | | GPPGEDGERGAEGPPGPTGQAGEPGPRGLLGPRGSPGPTGR | |
| | | PGVTGIDGAPGAKGNVGPPGEPGPPGQQGNHGSQGLPGPQG | |
| | | LIGTPGEKGPPGNPGIPGLPGSDGPLGHPGHEGPTGEKGAQ | |
| | | GPPGSAGPPGYPGPRGVKGTSGNRGLQGEKGEKGEDGFPGF | |
| | | KGDVGLKGDQGKPGAPGPRGEDGPEGPKGQAGQAGEEGPPG | |
| | | SAGEKGKLGVPGLPGYPGRPGPKGSIGFPGPLGPIGEKGKS | |
| | | GKTGQPGLEGERGPPGSRGERGQPGATGQPGPKGDVGQDGA | |
| | | PGIPGEKGLPGLQGPPGFPGPKGPPGHQGKDGRPGHPGQRG | |
| | | ELGFQGQTGPPGPAGVLGPQGKTGEVGPLGERGPPGPPGPP | |
| | | GEQGLPGLEGREGAKGELGPPGPLGKEGPAGLRGFPGPKGG | |
| | | PGDPGPTGLKGDKGPPGPVGANGSPGERGPLGPAGGIGLPG | |
| | | QSGSEGPVGPAGKKGSRGERGPPGPTGKDGIPGPLGPLGPP | |
| | | GAAGPSGEEGDKGDVGAPGHKGSKGDKGDAGPPGQPGIRGP | |
| | | AGHPGPPGADGAQGRRGPPGLFGQKGDDGVRGFVGVIGPPG | |
| | | LQGLPGPPGEKGEVGDVGSMGPHGAPGPRGPQGPTGSEGTP | |
| | | GLPGGVGQPGAVGEKGERGDAGDPGPPGAPGIPGPKGDIGE | |
| | | KGDSGPSGAAGPPGKKGPPGEDGAKGSVGPTGLPGDLGPPG | |
| | | DPGVSGIDGSPGEKGDPGDVGGPGPPGASGEPGAPGPPGKR | |
| | | GPSGHMGREGREGEKGAKGEPGPDGPPGRTGPMGARGPPGR | |
| | | VGPEGLRGIPGPVGEPGLLGAPGQMGPPGPLGPSGLPGLKG | |
| | | DTGPKGEKGHIGLIGLIGPPGEAGEKGDQGLPGVQGPPGPK | |
| | | GDPGPPGPIGSLGHPGPPGVAGPLGQKGSKGSPGSMGPRGD | |
| | | TGPAGPPGPPGAPAELHGLRRRRRFVPVPLPVVEGGLEEVL | |
| | | ASLTSLSLELEQLRRPPGTAERPGLVCHELHRNHPHLPDGE | |
| | | YWIDPNQGCARDSFRVFCNFTAGGETCLYPDKKFEIVKLAS | |
| | | WSKEKPGGWYSTFRRGKKFSYVDADGSPVNVVQLNFLKLLS | |
| | | ATARQNFTYSCQNAAAWLDEATGDYSHSARFLGTNGEELSF | |
| | | NQTTAATVSVPQDGCRLRKGQTKTLFEFSSSRAGFLPLWDV | |
| | | AATDFGQTNQKFGFELGPVCFSS | |
| COL6A2 | P12110 | MLQGTCSVLLLWGILGAIQAQQQEVISPDTTERNNNCPEKT | 14 |
| Collagen | | DCPIHVYFVLDTSESVTMQSPTDILLEHMKQFVPQFISQLQ | |
| alpha-2(VI) | | NEFYLDQVALSWRYGGLHFSDQVEVFSPPGSDRASFIKNLQ | |
| chain | | GISSERRGTFTDCALANMTEQIRQDRSKGTVHFAVVITDGH | |
| | | VTGSPCGGIKLQAERAREEGIRLFAVAPNQNLKEQGLRDIA | |
| | | STPHELYRNDYATMLPDSTEIDQDTINRIIKVMKHEAYGEC | |
| | | YKVSCLEIPGPSGPKGYRGQKGAKGNMGEPGEPGQKGRQGD | |
| | | PGIEGPIGFPGPKGVPGFKGEKGEFGADGRKGAPGLAGKNG | |
| | | TDGQKGKLGRIGPPGCKGDPGNRGPDGYPGEAGSPGERGDQ | |
| | | GGKGDPGRPGRRGPPGEIGAKGSKGYQGNSGAPGSPGVKGA | |
| | | KGGPGPRGPKGEPGRRGDPGTKGSPGSDGPKGEKGDPGPEG | |
| | | PRGLAGEVGNKGAKGDRGLPGPRGPQGALGEPGKQGSRGDP | |
| | | GDAGPRGDSGQPGPKGDPGRPGFSYPGPRGAPGEKGEPGPR | |
| | | GPEGGRGDFGLKGEPGRKGEKGEPADPGPPGEPGPRGPRGV | |
| | | PGPEGEPGPPGDPGLTECDVMTYVRETCGCCDCEKRCGALD | |
| | | VVFVIDSSESIGYINFTLEKNEVINVVNRLGAIAKDPKSET | |
| | | GTRVGVVQYSHEGTFEAIQLDDERIDSLSSFKEAVKNLEWI | |
| | | AGGTWTPSALKFAYDRLIKESRRQKTRVFAVVITDGRHDPR | |
| | | DDDLNLRALCDRDVTVTAIGIGDMFHEKHESENLYSIACDK | |
| | | PQQVRNMTLFSDLVAEKFIDDMEDVLCPDPQIVCPDLPCQT | |
| | | ELSVAQCTQRPVDIVELLDGSERLGEQNFHKARREVEQVAR | |
| | | RLTLARRDDDPLNARVALLQFGGPGEQQVAFPLSHNLTAIH | |
| | | EALETTQYLNSFSHVGAGVVHAINAIVRSPRGGARRHAELS | |
| | | FVFLTDGVTGNDSLHESAHSMRKQNVVPTVLALGSDVDMDV | |
| | | LTTLSLGDRAAVFHEKDYDSLAQPGEFDREIRWIC | |
| COL6A3 | P12111 | MRKHRHLPLVAVFCLFLSGFPTTHAQQQQADVKNGAAADII | 15 |
| Collagen | | FLVDSSWTIGEEHFQLVREFLYDVVKSLAVGENDFHFALVQ | |
| alpha-3(VI) | | FNGNPHTEFLLNTYRTKQEVLSHISNMSYIGGINQTGKGLE | |
| chain | | YIMQSHLTKAAGSRAGDGVPQVIVVLTDGHSKDGLALPSAE | |
| | | LKSADVNVFAIGVEDADEGALKEIASEPLNMHMENLENFTS | |
| | | LHDIVGNLVSCVHSSVSPERAGDTETLKDITAQDSADIIFL | |
| | | IDGSNNTGSVNFAVILDFLVNLLEKLPIGTQQIRVGVVQFS | |
| | | DEPRTMFSLDTYSTKAQVLGAVKALGFAGGELANIGLALDF | |
| | | VVENHFTRAGGSRVEEGVPQVLVLISAGPSSDEIRYGVVAL | |
| | | KQASVESEGLGAQAASRAELQHIATDDNLVETVPEFRSEGD | |
| | | LQEKLLPYIVGVAQRHIVLKPPTIVTQVIEVNKRDIVFLVD | |
| | | GSSALGLANFNAIRDFIAKVIQRLEIGQDLIQVAVAQYADT | |
| | | VRPEFYENTHPTKREVITAVRKMKPLDGSALYTGSALDFVR | |
| | | NNLFTSSAGYRAAEGIPKLLVLITGGKSLDEISQPAQELKR | |
| | | SSIMAFAIGNKGADQAELEEIAFDSSLVFIPAEFRAAPLQG | |
| | | MLPGLLAPLRTLSGTPEVHSNKRDIIFLLDGSANVGKINFP | |
| | | YVRDFVMNLVNSLDIGNDNIRVGLVQFSDTPVTEFSLNTYQ | |
| | | TKSDILGHLRQLQLQGGSGLNIGSALSYVYANHETEAGGSR | |
| | | IREHVPQLLLLLTAGQSEDSYLQAANALTRAGILTFCVGAS | |
| | | QANKAELEQIAFNPSLVYLMDDFSSLPALPQQLIQPLTTYV | |
| | | SGGVEEVPLAQPESKRDILFLEDGSANLVGQFPVVRDFLYK | |
| | | IIDELNVKPEGTRIAVAQYSDDVKVESRFDEHQSKPEILNL | |
| | | VKRMKIKTGKALNLGYALDYAQRYIFVKSAGSRIEDGVLQF | |
| | | LVLLVAGRSSDRVDGPASNLKQSGVVPFIFQAKNADPAELE | |
| | | QIVLSPAFILAAESLPKIGDLHPQIVNLLKSVHNGAPAPVS | |
| | | GEKDVVFLLDGSEGVRSGFPLLKEFVQRVVESLDVGQDRVR | |
| | | VAVVQYSDRTRPEFYLNSYMNKQDVVNAVRQLTLLGGPTPN | |
| | | TGAALEFVLRNILVSSAGSRITEGVPQLLIVLTADRSGDDV | |
| | | RNPSVVVKRGGAVPIGIGIGNADITEMQTISFIPDFAVAIP | |
| | | TFRQLGTVQQVISERVTQLTREELSRLQPVLQPLPSPGVGG | |
| | | KRDVVFLIDGSQSAGPEFQYVRTLIERLVDYLDVGFDTTRV | |
| | | AVIQFSDDPKVEFLLNAHSSKDEVQNAVQRLRPKGGRQINV | |
| | | GNALEYVSRNIFKRPLGSRIEEGVPQFLVLISSGKSDDEVD | |
| | | DPAVELKQFGVAPFTIARNADQEELVKISLSPEYVFSVSTF | |
| | | RELPSLEQKLLTPITTLTSEQIQKLLASTRYPPPAVESDAA | |
| | | DIVFLIDSSEGVRPDGFAHIRDFVSRIVRRLNIGPSKVRVG | |
| | | VVQFSNDVFPEFYLKTYRSQAPVLDAIRRLRLRGGSPLNTG | |
| | | KALEFVARNLFVKSAGSRIEDGVPQHLVLVLGGKSQDDVSR | |
| | | FAQVIRSSGIVSLGVGDRNIDRTELQTITNDPRLVFTVREF | |
| | | RELPNIEERIMNSFGPSAATPAPPGVDTPPPSRPEKKKADI | |
| | | VFLLDGSINFRRDSFQEVLRFVSEIVDTVYEDGDSIQVGLV | |
| | | QYNSDPTDEFFLKDFSTKRQIIDAINKVVYKGGRHANTKVG | |
| | | LEHLRVNHFVPEAGSRLDQRVPQIAFVITGGKSVEDAQDVS | |
| | | LALTQRGVKVFAVGVRNIDSEEVGKIASNSATAFRVGNVQE | |
| | | LSELSEQVLETLHDAMHETLCPGVTDAAKACNLDVILGFDG | |
| | | SRDQNVFVAQKGFESKVDAILNRISQMHRVSCSGGRSPTVR | |
| | | VSVVANTPSGPVEAFDFDEYQPEMLEKFRNMRSQHPYVLTE | |
| | | DTLKVYLNKFRQSSPDSVKVVIHFTDGADGDLADLHRASEN | |
| | | LRQEGVRALILVGLERVVNLERLMHLEFGRGFMYDRPLRLN | |
| | | LLDLDYELAEQLDNIAEKACCGVPCKCSGQRGDRGPIGSIG | |
| | | PKGIPGEDGYRGYPGDEGGPGERGPPGVNGTQGFQGCPGQR | |
| | | GVKGSRGFPGEKGEVGEIGLDGLDGEDGDKGLPGSSGEKGN | |
| | | PGRRGDKGPRGEKGERGDVGIRGDPGNPGQDSQERGPKGET | |
| | | GDLGPMGVPGRDGVPGGPGETGKNGGFGRRGPPGAKGNKGG | |
| | | PGQPGFEGEQGTRGAQGPAGPAGPPGLIGEQGISGPRGSGG | |
| | | AAGAPGERGRTGPLGRKGEPGEPGPKGGIGNRGPRGETGDD | |
| | | GRDGVGSEGRRGKKGERGFPGYPGPKGNPGEPGLNGTTGPK | |
| | | GIRGRRGNSGPPGIVGQKGDPGYPGPAGPKGNRGDSIDQCA | |
| | | LIQSIKDKCPCCYGPLECPVFPTELAFALDTSEGVNQDTFG | |
| | | RMRDVVLSIVNDLTIAESNCPRGARVAVVTYNNEVTTEIRF | |
| | | ADSKRKSVLLDKIKNLQVALTSKQQSLETAMSFVARNTFKR | |
| | | VRNGFLMRKVAVFFSNTPTRASPQLREAVLKLSDAGITPLF | |
| | | LTRQEDRQLINALQINNTAVGHALVLPAGRDLTDFLENVLT | |
| | | CHVCLDICNIDPSCGFGSWRPSFRDRRAAGSDVDIDMAFIL | |
| | | DSAETTTLFQFNEMKKYIAYLVRQLDMSPDPKASQHFARVA | |
| | | VVQHAPSESVDNASMPPVKVEFSLTDYGSKEKLVDFLSRGM | |
| | | TQLQGTRALGSAIEYTIENVFESAPNPRDLKIVVLMLTGEV | |
| | | PEQQLEEAQRVILQAKCKGYFFVVLGIGRKVNIKEVYTFAS | |
| | | EPNDVFFKLVDKSTELNEEPLMRFGRLLPSFVSSENAFYLS | |
| | | PDIRKQCDWFQGDQPTKNLVKFGHKQVNVPNNVTSSPTSNP | |
| | | VTTTKPVTTTKPVTTTTKPVTTTTKPVTIINQPSVKPAAAK | |
| | | PAPAKPVAAKPVATKMATVRPPVAVKPATAAKPVAAKPAAV | |
| | | RPPAAAAAKPVATKPEVPRPQAAKPAATKPATTKPMVKMSR | |
| | | EVQVFEITENSAKLHWERAEPPGPYFYDLTVTSAHDQSLVL | |
| | | KQNLTVTDRVIGGLLAGQTYHVAVVCYLRSQVRATYHGSFS | |
| | | TKKSQPPPPQPARSASSSTINLMVSTEPLALTETDICKLPK | |
| | | DEGTCRDFILKWYYDPNTKSCARFWYGGCGGNENKFGSQKE | |
| | | CEKVCAPVLAKPGVISVMGT | |
| COL6A5 | A8TX70 | MKILLIIFVLIIWTETLADQSPGPGPVYADVVFLVDSSDHL | 16 |
| Collagen | | GPKSFPFVKTFINKMINSLPIEANKYRVALAQYSDEFHSEF | |
| alpha-5(VI) | | HLSTFKGRSPMLNHLKKNFQFIGGSLQIGKALQEAHRTYFS | |
| chain | | APINGRDRKQFPPILVVLASAESEDEVEEASKALQKDGVKI | |
| | | ISVGVQKASEENLKAMATSHFHFNLRTIRDLSTFSQNMTQI | |
| | | IKDVTKYKEGAVDADMQVHFPISCQKDSLADLVFLVDESLG | |
| | | TGGNLRHLQTFLENIISSMDVKENCMRLGLMSYSNSAKTIS | |
| | | FLKSSTTQSEFQQQIKNLSIQVGKSNTGAAIDQMRRDGFSE | |
| | | SYGSRRAQGVPQIAVLVTHRPSDDEVHDAALNLRLEDVNVF | |
| | | ALSIQGANNTQLEEIVSYPPEQTISTLKSYADLETYSTKFL | |
| | | KKLQNEIWSQISTYAEQRNLDKTGCVDTKEADIHFLIDGSS | |
| | | SIQEKQFEQIKRFMLEVTEMFSIGPDKVRVGVVQYSDDTEV | |
| | | EFYITDYSNDIDLRKAIFNIKQLTGGTYTGKALDYILQIIK | |
| | | NGMKDRMSKVPCYLIVLTDGMSTDRVVEPAKRLRAEQITVH | |
| | | AVGIGAANKIELQEIAGKEERVSFGQNFDALKSIKNEVVRE | |
| | | ICAEKGCEDMKADIMFLVDSSWSIGNENFRKMKIFMKNLLT | |
| | | KIQIGADKTQIGVVQFSDKTKEEFQLNRYFTQQEISDAIDR | |
| | | MSLINEGTLTGKALNFVGQYFTHSKGARLGAKKFLILIIDG | |
| | | VAQDDVRDPARILRGKDVTIFSVGVYNANRSQLEEISGDSS | |
| | | LVFHVENFDHLKALERKLIFRVCALHDCKRITLLDVVFVLD | |
| | | HSGSIKKQYQDHMINLTIHLVKKADVGRDRVQFGALKYSDQ | |
| | | PNILFYLNTYSNRSAIIENLRKRRDTGGNTYTAKALKHANA | |
| | | LFTEEHGSRIKQNVKQMLIVITDGESHDHDQLNDTALELRN | |
| | | KGITIFAVGVGKANQKELEGMAGNKNNTIYVDNFDKLKDVF | |
| | | TLVQERMCTEAPEVCHLQEADVIFLCDGSDRVSNSDFVTMT | |
| | | TFLSDLIDNFDIQSQRMKIGMAQFGSNYQSIIELKNSLTKT | |
| | | QWKTQIQNVSKSGGFPRIDFALKKVSNMFNLHAGGRRNAGV | |
| | | PQTLVVITSGDPRYDVADAVKTLKDLGICVLVLGIGDVYKE | |
| | | HLLPITGNSEKIITFQDFDKLKNVDVKKRIIREICQSCGKT | |
| | | NCFMDIVVGFDISTHVQGQPLFQGHPQLESYLPGILEDISS | |
| | | IKGVSCGAGTEAQVSLAFKVNSDQGFPAKFQIYQKAVFDSL | |
| | | LQVNVSGPTHLNAQFLRSLWDTFKDKSASRGQVLLIFSDGL | |
| | | QSESNIMLENQSDRLREAGLDALLVVSLNTTAHHEFSSFEF | |
| | | GKRFDYRTHLTIGMRELGKKLSQYLGNIAERTCCCTFCKCP | |
| | | GIPGPHGTRGLQAMKGSQGLKGSRGHRGEDGNPGVRGDTGP | |
| | | QGDKGIAGCPGAWGQKGLKGFSGPKGGHGDDGIDGLDGEEG | |
| | | CHGFPGIKGEKGDPGSQGSPGSRGAPGQYGEKGFPGDPGNP | |
| | | GQNNNIKGQKGSKGEQGRQGRSGQKGVQGSPSSRGSRGREG | |
| | | QRGLRGVSGEPGNPGPTGTLGAEGLQGPQGSQGNPGRKGEK | |
| | | GSQGQKGPQGSPGLMGAKGSTGRPGLLGKKGEPGLPGDLGP | |
| | | VGQTGQRGRQGDSGIPGYGQMGRKGVKGPRGFPGDAGQKGD | |
| | | IGNPGIPGGPGPKGFRGLALTVGLKGEEGSRGLPGPPGQRG | |
| | | IKGMAGQPVYSQCDLIRFLREHSPCWKEKCPAYPTELVFAL | |
| | | DNSYDVTEESFNKTRDIITSIVNDLNIRENNCPVGARVAMV | |
| | | SYNSGTSYLIRWSDYNRKKQLLQQLSQIKYQDTTEPRDVGN | |
| | | AMRFVTRNVFKRTYAGANVRRVAVFFSNGQTASRSSIITAT | |
| | | MEFSALDISPTVFAFDERVFLEAFGFDNTGTFQVIPVPPNG | |
| | | ENQTLERLRRCALCYDKCFPNACIREAFLPEDSYMDVVFLI | |
| | | DNSRNIAKDEFKAVKALVSSVIDNFNIASDPLISDSGDRIA | |
| | | LLSYSPWESSRRKMGTVKTEFDFITYDNQLLMKNHIQTSFQ | |
| | | QLNGEATIGRALLWTTENLFPETPYLRKHKVIFVVSAGENY | |
| | | ERKEFVKMMALRAKCQGYVIFVISLGSTRKDDMEELASYPL | |
| | | DQHLIQLGRIHKPDLNYIAKFLKPFLYSVRRGFNQYPPPML | |
| | | EDACRLINLGGENIQNDGFQFVTELQEDFLGGNGFIGQELN | |
| | | SGRESPFVKTEDNGSDYLVYLPSQMFEPQKLMINYEKDQKS | |
| | | AEIASLTSGHENYGRKEEPDHTYEPGDVSLQEYYMDVAFLI | |
| | | DASQRVGSDEFKEVKAFITSVLDYFHIAPTPLTSTLGDRVA | |
| | | VLSYSPPGYMPNTEECPVYLEFDLVTYNSIHQMKHHLQDSQ | |
| | | QLNGDVFIGHALQWTIDNVFVGTPNLRKNKVIFVISAGETN | |
| | | SLDKDVLRNVSLRAKCQGYSIFVFSFGPKHNDKELEELASH | |
| | | PLDHHLVQLGRTHKPDWNYIIKFVKPFVHLIRRAINKYPTE | |
| | | DMKATCVNMTSPNPENGGTENTVLLLPGIYEIKTENGDLFD | |
| | | EFDSQAQHLLVLGNNHSSGSETATDLMQKLYLLFSTEKLAM | |
| | | KDKEKAHLEEISALVVDKQQEKEDKEMEATDI | |
| COL6A6 | A6NM27 | MMLLILFLVIICSHISVNQDSGPEYADVVFLVDSSDRLGSK | 17 |
| Collagen | | SFPFVKMFITKMISSLPIEADKYRVALAQYSDKLHSEFHLS | |
| alpha-6(VI) | | TFKGRSPMLNHLRKNFGFIGGSLQIGKALQEAHRTYFSAPA | |
| chain | | NGRDKKQFPPILVVLASSESEDNVEEASKALRKDGVKIISV | |
| | | GVQKASEENLKAMATSQFHFNLRTVRDLSMFSQNMTHIIKD | |
| | | VIKYKEGAVDDIFVEACQGPSMADVVFLLDMSINGSEENFD | |
| | | YLKGFLEESVSALDIKENCMRVGLVAYSNETKVINSLSMGI | |
| | | NKSEVLQHIQNLSPRTGKAYTGAAIKKLRKEVFSARNGSRK | |
| | | NQGVPQIAVLVTHRDSEDNVTKAAVNLRREGVTIFTLGIEG | |
| | | ASDTQLEKIASHPAEQYVSKLKTFADLAAHNQTFLKKLRNQ | |
| | | ITHTVSVFSERTETLKSGCVDTEEADIYLLIDGSGSTQATD | |
| | | FHEMKTFLSEVVGMFNIAPHKVRVGAVQYADSWDLEFEINK | |
| | | YSNKQDLGKAIENIRQMGGNTNTGAALNFTLSLLQKAKKQR | |
| | | GNKVPCHLVVLTNGMSKDSILEPANRLREEHIRVYAIGIKE | |
| | | ANQTQLREIAGEEKRVYYVHDFDALKDIRNQVVQEICTEEA | |
| | | CKEMKADIMFLVDSSGSIGPENFSKMKTFMKNLVSKSQIGP | |
| | | DRVQIGVVQFSDINKEEFQLNRFMSQSDISNAIDQMAHIGQ | |
| | | TTLTGSALSFVSQYFSPTKGARPNIRKFLILITDGEAQDIV | |
| | | KEPAVVLRQEGVIIYSVGVFGSNVTQLEEISGRPEMVFYVE | |
| | | NFDILQRIEDDLVFGICSPREECKRIEVLDVVFVIDSSGSI | |
| | | DYDEYNIMKDFMIGLVKKADVGKNQVRFGALKYADDPEVLF | |
| | | YLDDFGTKLEVISVLQNDQAMGGSTYTAEALGFSDHMFTEA | |
| | | RGSRLNKGVPQVLIVITDGESHDADKLNATAKALRDKGILV | |
| | | LAVGIDGANPVELLAMAGSSDKYFFVETFGGLKGIFSDVTA | |
| | | SVCNSSKVDCEIDKVDLVFLMDGSTSIQPNDFKKMKEFLAS | |
| | | VVQDFDVSLNRVRIGAAQFSDTYHPEFPLGTFIGEKEISFQ | |
| | | IENIKQIFGNTHIGAALREVEHYFRPDMGSRINTGTPQVLL | |
| | | VLTDGQSQDEVAQAAEALRHRGIDIYSVGIGDVDDQQLIQI | |
| | | TGTAEKKLTVHNFDELKKVNKRIVRNICTTAGESNCFVDVV | |
| | | VGFDVSTQEKGQTLLEGQPWMETYLQDILRAISSLNGVSCE | |
| | | VGTETQVSVAFQVTNAMEKYSPKFEIYSENILNSLKDITVK | |
| | | GPSLLNANLLDSLWDTFQNKSAARGKVVLLFSDGLDDDVEK | |
| | | LEQKSDELRKEGLNALITVALDGPADSSDLADLPYIEFGKG | |
| | | FEYRTQLSIGMRELGSRLSKQLVNVAERTCCCLFCKCIGGD | |
| | | GTMGDPGPPGKRGPPGFKGSEGYLGEEGIAGERGAPGPVGE | |
| | | QGTKGCYGTKGPKGNRGLNGQEGEVGENGIDGLNGEQGDNG | |
| | | LPGRKGEKGDEGSQGSPGKRGTPGDRGAKGLRGDPGAPGVD | |
| | | SSIEGPTGLKGERGRQGRRGWPGPPGTPGSRRKTAAHGRRG | |
| | | HTGPQGTAGIPGPDGLEGSLGLKGPQGPRGEAGVKGEKGGV | |
| | | GSKGPQGPPGPGGEAGNQGRLGSQGNKGEPGDLGEKGAVGF | |
| | | PGPRGLQGNDGSPGYGSVGRKGAKGQEGFPGESGPKGEIGD | |
| | | PGGPGETGLKGARGKMISAGLPGEMGSPGEPGPPGRKGVKG | |
| | | AKGLASFSTCELIQYVRDRSPGRHGKPECPVHPTELVFALD | |
| | | HSRDVTEQEFERMKEMMAFLVRDIKVRENSCPVGAHIAILS | |
| | | YNSHARHLVRFSDAYKKSQLLREIETIPYERSSASREIGRA | |
| | | MRFISRNVFKRTLPGAHTRKIATFFSSGQSADAHSITTAAM | |
| | | EFGALEIIPVVITFSNVPSVRRAFAIDDTGTFQVIVVPSGA | |
| | | DYIPALERLQRCTFCYDVCKPDASCDQARPPPVQSYMDAAF | |
| | | LLDASRNMGSAEFEDIRAFLGALLDHFEITPEPETSVTGDR | |
| | | VALLSHAPPDFLPNTQKSPVRAEFNLTTYRSKRLMKRHVHE | |
| | | SVKQLNGDAFIGHALQWTLDNVFLSTPNLRRNKVIFVISAG | |
| | | ETSHLDGEILKKESLRAKCQGYALFVFSLGPIWDDKELEDL | |
| | | ASHPLDHHLVQLGRIHKPDHSYGVKFVKSFINSIRRAINKY | |
| | | PPINLKIKCNRLNSIDPKQPPRPFRSFVPGPLKATLKEDVL | |
| | | QKAKFFQDKKYLSRVARSGRDDAIQNFMRSTSHTFKNGRMI | |
| | | ESAPKQHD | |

Fibrillar collagenous ECM collagen, a kind of mature ECM in accordance with methods, compositions, and kits of some embodiments herein, can include COL1 a major component, and can also comprise COL3, COL4, COL5, and/or COL6, for example about 90% COL1 and about 10% COL3, COL4, COL5, and/or COL6, though other percentages of (i) COL1 and (ii) COL3, COL4, COL5 and/or COL6, are suitable, for example about 97% and 3% respectively, about 95% and 5% respectively, about 93% and 7% respectively, about 85% and 15% respectively, about 80% and 20% respectively, about 75% and 25% respectively, or about 70% and 30%, respectively.

ECM, and in particular, human ECM products as produced according to methods, compositions, and kits of some embodiments herein are useful for treating tissues in patients suffering from musculoskeletal disorders, orthopedic dysfunction and associated pain, cardiovascular disorders, cutaneous diseases, age-related cosmetic skin and hair conditions requiring improvement in appearance, surgical wounds, solid-tumors requiring treatment including surgical excision, chemotherapy and immunotherapy. Uses for the human ECM products produced by methods, kits, and compositions in accordance with some embodiments herein, by way of example, include medical devices and biologics for musculoskeletal applications including osteochondral defect repair, osteoarthritis, degenerative disc-disease, surgical wounds for orthopedics; surgical wounds associated with tumor resection cavities, cardiovascular regeneration devices and biologics, cutaneous wound devices and biologics, dermal fillers for treating wrinkles, topical cosmetics, therapeutic hair growth, and cell-delivery and drug-delivery vehicles for increased persistence and reduction of unwanted immune reactions when transferred to a patient. In some embodiments, ECM (e.g., a human ECM product) produced according to methods, compositions, and kits of some embodiments herein is used for at least one of a medical product, a cosmetic product, a drug, a medical device, a treatment, or a biologic. In some embodiments the ECM can be used for at least one of a medical product, a biologic, a medical device, a drug, or any other product or composition regulated by the FDA. In some embodiments, the ECM can be used for at least one of a medical or cosmetic procedure.

The use of human cell cultures for the production of human ECM in accordance with methods, compositions, and kits of some embodiments herein can offer advantages over animal and cadaver-derived ECMs. For example, in some embodiments, the cells in culture can be from a single donor, can be readily expanded, and/or can be xeno-free. On the other hand, conventional approaches for producing ECM in cell culture presented challenges, for example challenges related to commercial scale, cost-effectiveness, and the presence of non-human components. For example, fibroblast-derived human ECMs from cell culture and methods of manufacture have been described. For example, a group of patents by Naughton, et al (U.S. Pat. Nos. 6,378,527; 5,830,708; 7,118,746; 6,372,494; 8,257,947; 8,530,415; 8,535,913; 9,034,312; 8,852,637; 8,128,924; 8,138,147; 8,361,485; 8,476,231; and 9,458,486, referred to as the “Naughton patents,” and hereby incorporated by reference in their entireties) disclose various compositions and methods of production for soluble and insoluble human ECM. For example, the Naughton patents disclose the use of animal proteins such as fetal bovine serum, calf serum, and porcine trypsin to support cell expansion and production of human ECM. These processes using animal components have also utilized porcine pepsin to collagens after the ECM is produced, for example in the manufacture of CosmoDerm™ and CosmoPlast™ dermal filler products. For example, porcine carbohydrates such as porcine heparin can cause deleterious immune reactions in humans.

The Naughton patents also disclose soybean trypsin inhibitor. Without being limited by theory, soybean trypsin inhibitor can be used when serum is not included to reduce deleterious protein degradation in cell cultures. However, some plant proteins, and soybean trypsin inhibitor in particular, can cause unwanted immune reactions in humans, even potentially allergies or anaphylactic shock or death in rare cases. As such, it is contemplated that replacement of enzyme inhibitors found in animal sera with plant components, such as soybean trypsin inhibitor can still cause unwanted immune reactions. Furthermore, it is contemplated that soybean trypsin inhibitor may possess enzymatic activity, which can affect compositions that contain soybean trypsin inhibitor, or skin tissue to which the soybean trypsin inhibitor is exposed, for example upon topical or transdermal application. Soybean trypsin inhibitor has also been shown to inhibit hair growth. While removing the soluble soybean trypsin inhibitor is formally possible, doing so is of limited commercial practicality as it would involve extensive and costly purification methods in an undefined mixture of cell-secreted products in spent culture medium, yielding products in which the exact active ingredients can be unknown. As such, some embodiments do not include animal-derived products, or soybean trypsin inhibitor. Some embodiments do not include animal-derived products or plant-derived products.

Advantageously, xeno-free methods of manufacturing ECM in accordance with some embodiments herein can obviate the need for clinical studies directed to removal of animal-and/or plant-) derived components, as the xeno-free systems and methods can avoid the use of animal- and/or plant-derived components in the first place. For example, methods and compositions in accordance with some embodiments herein can produce soluble ECM compositions for cosmetic uses, and which do not necessarily contain known plant allergens such as soy proteins. Additionally, ECM compositions manufactured in accordance with some embodiments herein that do not contain animal- or plant-derived components will not require safety warnings for potential allergies to animal products or plant products such as soy proteins.

In some embodiments, cell-based in vitro culture methods can produce xeno-free human ECM at scales larger than a single human per batch. Furthermore, the xeno-free ECM can have a reduced risk of disease transmission since the cell lines can be extensively tested, and each set of tests can support many commercial-scale batches of manufactured products using human ECM, because the tested cell lines can be expanded.

Fibroblast spent medium can comprise soluble ECM, and thus can also be used for cosmetic purposes in some embodiments. Without being limited by theory, it is observed herein that fetal and embryonic ECM contains relatively lower degrees of abundance of mature and non-reducible cross-links. However, soluble human ECM produced in accordance with some embodiments herein can comprise a greater amount of mature collagen type 1, for example a greater amount of mature triple-helical collagen type 1, as well as some less abundant fibrillar collagens, for example collagen types 3, 5, and 6.

A number of approaches can be used to assess the presence and/or levels of mature collagens in ECM in accordance with embodiments herein. For example, amounts of cross-linked mature collagens can be measured on reduced. SDS-PAGE gels, using adult-tissue derived type I collagen for comparison.

### Pluripotent Cells

“Pluripotent cell” is used herein in accordance with its ordinary meaning in the field, and includes a class of cells that are capable of differentiating according to multiple different fates. Examples of pluripotent cells include, but are not limited to, Induced Pluripotent Stem Cells (iPSCs) and embryonic stem (ES) cells. In some embodiments, pluripotent cells suitable for methods, compositions, and/or kits herein comprise, consist of, or consist essentially of iPSCs. In some embodiments, pluripotent cells suitable for methods, compositions, and kits herein comprise, consist of, or consist essentially of iPSCs or ES cells. In some embodiments, pluripotent cells suitable for methods, compositions, and kits herein comprise, consist of, or consist essentially of iPSCs, but not ES cells. In some embodiments, pluripotent cells suitable for methods, compositions, and/or kits herein are human cells. In some embodiments, pluripotent cells suitable for methods, compositions, and/or kits herein are from a single donor.

Without being limited by theory, pluripotent cells (induced or embryonic in origin) conventionally did not produce enough fibrillar collagenous ECM in cell cultures. However, in methods, compositions, and kits of some embodiments herein, pluripotent cells can provide an abundant supply (which can be thought of as nearly limitless) of cells from a single donor, and can be used when they are differentiated into fibroblastic cells that secrete and deposit ECM. Thus, de-differentiating fibroblasts into iPSCs, expanding the iPSCs, and then re-differentiating the iPSCs into fibroblasts in accordance with compositions, methods, and kits of some embodiments herein can produce commercial-scale quantities of single-donor fibroblasts for the production of ECM. As the cells are single-donor, there can be greater uniformity in the ECM product produced, and lower risk for contamination and/or transmission of diseases. The skilled artisan would appreciate that generically producing iPSC's from fibroblasts, and then differentiating the iPSC's back into fibroblasts would involve a considerable amount of effort, but that the practical advantages discussed herein for the practical uses discussed herein would make the effort worthwhile.

“Induced Pluripotent Stem Cell” (iPSC) is used herein in accordance with its ordinary meaning in the field, and includes a class of cells produced by de-differentiation of somatic cells into cells having similar characteristics as ES cells. A number of art-recognized approaches for making iPSCs are suitable for methods, kits, and compositions of embodiments herein. In some embodiments, an iPSC is made by contacting a somatic cell with de-differentiation factors. As noted in detail herein, de-differentiation factors can comprise nucleic acids, polypeptides, and/or small molecules that induce a somatic cell to de-differentiate into an iPSC.

As used herein “de-differentiation factors” (including variants of this root term) refers to a set of gene products, and/or nucleic acids encoding gene products, and/or small molecules, which are sufficient to de-differentiate a somatic cell (e.g., a fibroblast) into an iPSC. De-differentiation factors can be used to de-differentiate a somatic cell (e.g. a fibroblast) into an iPSC in accordance with methods of some embodiments herein. De-differentiation factors can also be included with compositions and kits in accordance with some embodiments herein, as they can be useful for de-differentiating a somatic cell (e.g., a fibroblast) into an iPSC. In some embodiments, the de-differentiation factors comprise two or more transcription factors. In some embodiments, the de-differentiation factors comprise, consist essentially of, or consist of an Oct family member (e.g., Oct3/4 or POU5F1), a Sox family member (e.g., Sox1, Sox2, Sox3, Sox4, Sox11, or Sox15), a Klf family member (e.g., Klf1, Klf2, Klf4, or Klf5), and least one of (i) a Myc family member (e.g., c-Myc, L-Myc, or N-Myc), (ii) Nanog, (iii) Lin28 or Lin28B or (iv) Glis1, and/or (in the case of de-differentiated fibroblasts, an Oct family member (e.g., Oct 4) and Bmi1. In some embodiments, the de-differentiation factors comprise, consist essentially of, or consist of chemical de-differentiation factors (e.g., small molecules).

In some embodiments, the de-differentiation factors are provided as one or more nucleic acids that encode gene products sufficient to de-differentiate a somatic cell (e.g., a fibroblast) into an iPSC. Such de-differentiation factors can be provided on a single vector, or on a set of more than one vectors. Examples of suitable vectors in accordance with methods, compositions, and kits of some embodiments include, but are not limited to, retroviral vectors, adenoviral vectors, adeno-associated vectors, lentiviral vectors, and the like. In some embodiments, each of the nucleic acids encoding the de-differentiation factors is operably linked to a promoter (it is also contemplated that two or more nucleic acids can be under the control of the same promoter, for example, separated by an IRES or 2A element). In some embodiments, the de-differentiation factors are provided as one or more gene products (e.g. proteins) sufficient to de-differentiate a somatic cell (e.g., a fibroblast into an iPSC). In some embodiments, the de-differentiation factors are provided as a collection of proteins. In some embodiments, the de-differentiation factors are provided in a single polypeptide, which can be cleaved to yield individual de-differentiation factors. The polypeptide(s) can further include a tag, for example, a nuclear localization sequence, to facilitate entry and localization, into a suitable portion of a target cell. In some embodiments, the de-differentiation factors comprise chemical de-differentiation factors, as described herein. There can be additional advantages associated with “footprint free” de-differentiation factors (for example delivered by non-integrating vectors, removal of vectors, direct administration of mRNA or polypeptides, or chemical induction of pluripotency), since they do not use potentially harmful viruses to deliver factors that induce pluripotency, and do not insert foreign material into the host genome, which can raise a risk of insertional mutagenesis. Accordingly, in some embodiments, the de-differentiation factors are footprint-free. Examples of footprint-tree generation of iPSCs from somatic cells by delivery of mRNAs encoding Klf4, c-Myc, Oct4, and Sox2 to the somatic cells can be found, for example, in Warren et al. (2010), Cell Stem Cell 7: 618-30, which is hereby incorporated by reference in its entirety such, in some embodiments, mRNAs encoding de-differentiation factors are used to make iPSCs. In some embodiments, chemical induction of pluripotency is as described herein.

It has been reported that a combination of an Oct family member (e.g., Oct3, Oct4, /or POU5F1) and a Sox family member (e.g., Sox1, Sox2, Sox3, Sox4, Sox11, or Sox15) are sufficient to de-differentiate a somatic cell into an iPSC. See U.S. Pat. No. 9,683,232, which is hereby incorporated by reference in its entirety. As such, in some embodiments, the de-differentiation factors comprise, consist essentially of, or consist of an Oct family member, and a Sox family member. See U.S. Pat. No. 9,683,232. Furthermore, without being limited by theory, it is contemplated that the inclusion of additional factors can increase the efficiency of de-differentiation. For example, in some embodiments, the de-differentiation factors comprise, consist essentially of, or consist of an Oct family member, a Klf family member (e.g., Klf1, Klf2, Klf4, or Klf5) and a Sox family member. For example, the combination of Oct 3/4, Klf4, c-Myc, and Sox2 is sufficient to de-differentiate somatic cells (and fibroblasts in particular) into iPSCs. See U.S. Pat. No. 8,058,065, which is hereby incorporated by reference in its entirety. Accordingly, in some embodiments, the de-differentiation factors comprise, consist essentially of, or consist of Oct 3/4 Klf4, c-Myc, and Sox2.

In some embodiments, the de-differentiation factors comprise, consist essentially of, or consist of an Oct family member (e.g., Oct3, Oct4, or POU5F1), a Klf family member (e.g., Klf1, Klf2, Klf4, or Klf5), a Myc family member (e.g., c-Myc, L-Myc, or N-Myc), and a Sox family member (e.g. Sox1, Sox2, Sox3, Sox4, Sox11, or Sox15). Furthermore, it has been reported that a combination of Oct3/4, Klf4, Sox2, and at least one of (i) a Myc family member, (ii) Nanog, (iii) Lin28 or Lin28B, or (iv) Glis1 is also sufficient to de-differentiate somatic cells, and fibroblasts in particular, into iPSCs. See U.S. Pat. No. 9,447,408, which is hereby incorporated by reference in its entirety. Accordingly, in some embodiments, the de-differentiation factors comprise, consist essentially of, or consist of Oct3/4, Klf4, Sox2, and at least one of (i) a Myc family member, (ii) Nanog, (iii) Lin28 or Lin28B, or (iv) Glis1.

It has also been reported that fibroblasts in particular can be reprogrammed into iPSCs using a combination of the factors Oct4 and Bmi1 (e.g., so that Bmi1 can be substituted for the combination of Sox2, Klf4, and/or c-Myc). Moon et al. (2011), Cell. Res. 21: 1305-15, which is hereby incorporated by reference in its entirety. Accordingly, in some embodiments, for example if a precursor fibroblast is de-differentiated into an iPSC, the de-differentiation factors comprise, consist essentially of, or consist of Oct4 and Bmi1.

It has been shown that a cocktail of small molecules, “VC6TF” (V, VPA; C, CHIR99021 or CHIR; 6, 616452; T, tranylcypromine; F, forskolin) can induce pluripotent stem cells from somatic cells. Hou et al. (2013), Science 341: 651-654, which is hereby incorporated by reference in its entirety. For example, the addition of EPZ 004777 (EPZ, E), an inhibitor of H3K79 histone methyltransferase DOT1L, and Ch 55, a retinoid acid receptor (RAR) agonist to VC6TF has been shown to boost de-differentiation of somatic cells. Ye et al. (2016), Cell Research 26: 34-35, which is hereby incorporated by reference in its entirety. Accordingly, in some embodiments, the de-differentiation factors comprise, consist of, or consist essentially of chemical de-differentiation factors, for example, VC6TF, or VC6TF along with EPZ 004777, DOT1L, and Ch 55.

In some embodiments, the iPSCs are derived from somatic cells of a single donor. Without being limited by theory, iPSCs (or other pluripotent, cells) from a single donor can offer safety advantages, for example limiting the exposure of the cells to only a single donor's complement of viruses, microbial organisms, or other potential pathogens, and thus minimizing the risk for transmission of disease compared to collections of cells from multiple donors. It is also formally possible for the iPSCs, in some embodiments, to be derived from somatic cells of two or more donors. In some embodiments, the iPSCs are derived from adult dermal fibroblast biopsies.

Embryonic stem cells are another type of pluripotent stem cell. It, is possible to use embryonic stem cells as pluripotent cells in the methods, compositions, and kits of some embodiments herein. Methods of isolating and preparing embryonic stem cells, including human embryonic stem cells are described, for example, in U.S. Pat. No. 6,200,806, which is hereby incorporated by reference in its entirety. However, it is also recognized that iPSC's in some embodiments can offer a number of advantages over embryonic stem cells. For example, ES cells may have limitations involving ethical and legal considerations around the destruction of a human embryo, which is one way to generate a pluripotent cell. Chemically-induced pluripotent cells are the preferred source.

### Fibroblasts

“Fibroblast” is used herein in accordance with its ordinary meaning in the field, and includes a class of cells that provide structural framework (stroma) for a variety of animal tissues. Fibroblasts can also migrate at the site of a wound to mediate wound healing, and can be a component in a variety of connective tissues. Fibroblasts can be identified, for example, using fibroblast-specific antibodies, for example antibody TE-7 described in Goodpaster et al. (2008), J. Histochem Cyotchem. 56: 347-58, which is hereby incorporated by reference in its entirety.

Without being limited by theory, it is contemplated that any mesenchymal cell that can adhere to a substrate (e.g., a plastic), grow in the presence of serum, and synthesize and deposit fibrillar collagenous ECM collagen in cell culture can be used for making human ECM in culture. In some embodiments, fibroblasts, a type of mesenchymal cell, are used to produce ECM. Example types of cells that can produce ECM (which may be referred to as “ECM-producing cells”) include, but are not limited to, a bone marrow mesenchymal stem cell (MSC), an iPSC-derived MSC, an ES-derived MSC, and a fibroblast, for example from a neonatal foreskin fibroblast cell line (which can be multipotent or non-multipotent), or from a skin or blood biopsy. Without being limited by theory, it is further contemplated that differences between mesenchymal cell types may be no greater than those between any cells of a single donor strain or cell line (from a common cell source). If the cells come from a human donor sample that contains connective tissues, these cells can be suitable for making ECM in cell culture in accordance with some embodiments herein.

Conventionally, fibroblasts have been obtained from neonatal foreskins, since these are easily obtained from discarded tissues. However, this approach generally requires cells from multiple donors in order to generate and screen production-scale cell banks. The use of more than one donor can also raise an increased risk for transmission of adventitious agents. The costs of developing and screening donor cell banks also can be large and disadvantageous. Adventitious agent testing requirements can represent another hurdle for banking and using cells from multiple donors. Furthermore, while primary cell lines can be limited theoretically by the Hayflick limit for non-transformed somatic cells, practically speaking, primary cell lines may be amenable to no more than about 8-20 passages after isolation from the tissue source. These limits on passages can limit the suitability of conventional fibroblasts for expansions in line with a cell banking process, for example that of a Master Cell Bank, Working Cell Bank, or Production Cell Bank schema for manufacturing commercial quantities. However, methods, kits, and compositions in accordance with some embodiments herein can offer advantages over conventional sources of fibroblast. For example, fibroblasts differentiated from iPSCs in accordance with some embodiments herein can be from a single donor (e.g., if iPSCs are expanded before they are differentiated into fibroblasts), reducing the risk for transmission of disease, and reducing testing requirements. Furthermore, in some embodiments, iPSCs can undergo many more cycles of expansion than primary cell lines, facilitating the commercial scaleability of fibroblasts or other ECM-producing cells derived from iPSCs. In some embodiments, the iPSCs are expanded in the absence of serum.

In some embodiments, fibroblasts can be obtained by differentiating iPSCs into fibroblasts. Without being limited by theory, it is contemplated that iPSCs can be differentiated into fibroblasts by contacting the iPSCs with one or more fibroblast differentiation factor, which can include, for example, one or more growth factors. As used herein “fibroblast differentiation factors” (including variants of this root term) refer to a set of gene products and/or nucleic acids encoding gene products, in which the gene products are sufficient to differentiate a pluripotent cell (e.g. an iPSC) into a fibroblast. For example, it has been reported that iPSCs can be differentiated into fibroblasts by contacting the iPSCs with connective tissue growth factor (CTGF). The iPSC's can be grown on a 3-D scaffold. Xu et al., Scientific Reports 5: 8480 DOI: 10.1038/srep08480, which is hereby incorporated by reference in its entirety. As such, in some embodiments, the fibroblast differentiation factors comprise, consist essentially of, or consist of CTGF. In some embodiments, the fibroblast differentiation factors comprise, consist essentially of, or consist of CTGF and fibrinogen. In some embodiments, the iPSCs are cultured on a 3-dimensional substrate (for example, a dextrin microcarriers; see, e.g., U.S. Pat. No. 6,378,527, which is hereby incorporated by reference in its entirety), and then contacted with the fibroblast differentiation factors.

As used herein “production fibroblasts” refer to fibroblasts that are being used, or can be used for the production of ECM in accordance with methods, compositions, and kits of some embodiments herein.

As used herein, “precursor fibroblasts” refer to fibroblasts that are being used, or can be used, a precursors to Pluripotent cells. For example, in accordance with methods and kits of some embodiments, a precursor fibroblast can be contacted with de-differentiation factors, so as to de-differentiate the precursor fibroblast into an iPSC.

### Cell Culture and Substrates

A variety of approaches for cell culture can be used in accordance with methods, kits, and compositions of some embodiments herein. Detailed guidance on protocols and reagents for cell culture can be found, for example, in Sambrook et al., “Molecular Cloning: A Laboratory Manual (Third ed.), Cold Spring Harbor Laboratory Press, Cold Spring Harbor, N.Y., 2000, which is hereby incorporated by reference in its entirety. Generally, culturing fibroblasts for the manufacture of ECMs according to methods, compositions, and kits of some embodiments herein can be performed in culture medium.

In some embodiments, the cell culture medium comprises serum. The serum can be part of the cell culture medium initially, or can be added later in the culture process. Suitable types of serum for use with methods, compositions, and kits of some embodiments herein include tested clinical-grade bovine calf serum, pooled human serum from expired blood units, or combinations of these two substances. In some embodiments, the amount of serum in the culture medium (v/v) is about 0.1% to about 20%, for example about 0.1% to about 15%, about 0.1% to about 10%, about 0.1% to about 5%, about 0.1% to about 3%, about 0.1% to about 1%, about 1% to about 20%, about 1% to about 15%, about 1% to about 10%, about 1% to about 5%, about 1% to about 3%, about 3% to about 20%, about 3% to about 35%, about 3% to about 30%, about 3% to about 5%, about 5% to about 20%, about 5% to about 15%, about 5% to about 10%, about 10% to about 20%, or about 15% to about 20%, for example about 0.1%, 0.5% , 2%, 3%, 4%, 5%, 6%, 7%, 8% , 9%, 10%, 11%, 12%, 13%, 14%, 15%, 16%, 17%, 18%, 19%, or 20%, including ranges between any two of the listed values. In some embodiments, the amount of serum in the culture medium (v/v) is about 0.1% to about 10%. In some embodiments, the amount of serum in the culture medium is an amount sufficient to increase bioproduction of ECM by cells in the culture. In some embodiments, the amount of serum in the culture medium is an amount sufficient to induce maturation and crosslinking of ECM produced by cells in the culture. As used here: “crosslinked” ECM is used herein in accordance with its ordinary meaning in the field, and includes ECM for which polypeptides (e.g. collagen) are directly or indirectly bound one or more other polypeptides of the ECM by an ionic bond and/or covalent bond other than a peptide linkage. In some embodiments, each polypeptide of crosslinked ECM is covalently and/or ionically bound to at least one other polypeptide of the crosslinked ECM by a bond that is not a peptide bond. In some embodiments, a crosslinked peptide of the ECM is directly bound to another peptide of the ECM. In some embodiments, a crosslinked polypeptide of the ECM is indirectly bound to another polypeptide of the ECM, for example via an intervening small molecule, an ion, an amino acid, and/or a different polypeptide. In some embodiments, crosslinked ECM includes ECM in which a majority, substantially all, or all, of the polypeptides are bound by a non-peptide bond at least one other polypeptide of the ECM, for example about or at least about 50%, 60%, 65%, 70%, 75%, 80%, 85%, 90%, 95%, 97%, or 99% of the polypeptides, including ranges between any two of the listed values.

In some embodiments, pluripotent cells (e.g., iPSCs) or fibroblasts are cultured on or near a substrate. In some embodiments, the substrate comprises, consists of, or consists essentially of dextran, for example dextran microcarriers, Advantageously, dextran substrates can subsequently be digested using dextranase as described herein, which can facilitate isolation and purification of ECM produced by cell culture in accordance with some embodiments herein. In some embodiments, the substrate comprises, consists of, or consists essentially of a carbohydrate. In some embodiments, the substrate comprises, consists of, or consists essentially of a polymer. In some embodiments, the substrate comprises, consists of, or consists essentially of a plastic.

Dextran is a large, branched carbohydrate made out of many glucose molecules. Dextran chains can be of varying lengths, for example having molecular weights from as few as 3 kDa to more than 2000 kDa. It is contemplated that dextran structures such as dextran microcarriers can be used as a substrate for the culture of cells, such as pluripotent cells, or fibroblasts in methods, kits, and compositions of some embodiments herein. Advantageously, dextran can be non-toxic, and can readily be hydrolyzed by dextranase as described in more detail herein.

Dextranase is a bacterial enzyme widely used in industrial applications (EC 3.2.1.11, dextran hydrolase, endodextranase, dextranase DL2, DL 2, endo-dextranase, alpha-D-1,6-glucan-6-glucanohydrolase, 1,6-alpha-D-glucan 6-glucanohydrolase), and has the systematic name 6-alpha-D-glucan 6-glucanohydrolase. This enzyme catalyses the following chemical reaction: endohydrolysis of (1->6)-alpha-D-glucosidic linkages in dextran. Dextranase in cell culture can be used for a variety of applications, for example isolation of expanded mammalian cells, such as human chondrocytes, and mammalian cell lines for manufacturing cells and cell-derived virus or other biological products on dextran microcarriers (See, e.g., U.S. Pat. No. 6,378,527), or for waste-disposal of the massive volumes of beads used in commercial-scale cell cultures for biologics production. In methods, kits, and compositions of some embodiments, dextran can be used as a substrate or scaffold for pluripotent cells (for example iPSCs), or for fibroblasts derived from pluripotent cells (e.g., fibroblasts produced by de-differentiating a fibroblast into an iPSC, and then re-differentiating the iPSC into a fibroblast). In some embodiments, the dextran is not used for culture and/or expansion of chondrocytes. In some embodiments, the dextran is not used for culture of non--human cells, for example VERO (simian) and/or CHO (rodent) cells.

The use of dextranase enzyme on a variety of research-grade, small scale cultures, generally not applicable for commercial use, human ECM is disclosed by Pinney et al (See Pinney E, (2011) International Journal of Stem Cells 4: 70-75; and Menen et al., (2012) Anticancer Research 32: 1573-1578, each of which is hereby incorporated by reference in its entirety). It is noted that conventional approaches have involved the use of dextran for cell isolation after expansion, primarily in the context of waste disposal of dextran microcarriers. In some embodiments, dextran is used for the culture of pluripotent cells or fibroblasts that produce ECM. In some embodiments, the dextran is used in a culture of pluripotent cells or fibroblasts, but is not used for waste disposal. In some embodiments, the culture comprises human ECM produced by the pluripotent cells or fibroblasts.

In the methods, compositions, and/or kits of some embodiments, ECM is manufactured by cells in culture (e.g., fibroblasts, and/or iPSCs), and then the ECM is isolated from the cells in culture. In some embodiments, isolating the ECM comprises isolating a soluble fraction of ECM. Spent medium comprising soluble ECM can be isolated from cell cultures, and optionally, can be replaced by fresh medium so that the cell cultures can continue to produce. In some embodiments the ECM is isolated by an acidic wash, followed by dextranase digestion at a pH that facilitates the enzymatic activity of dextranase (e.g., pH 6.0 to 6.5). The dextranase can be at a relatively low concentration (less than 1000 U/ml, for example less than 1000 U/ml, 900 U/ml, 800 U/ml, 700 U/ml, 600 U/ml, 500 U/ml, 400 U/ml, 300 U/ml, 200 U/ml, 100 U/ml, 50 U/ml, 10 U/ml, 5 U/ml, 2 U/ml, or 1 U/ml, including ranges between any 2 of these values, for example 1-1000 U/ml; 500-1000 U/ml, 1-500 U/ml, 100-300 U/ml, or 600-800 U/ml). It is contemplated that the lower concentrations of dextranase are less likely to non-specifically degrade other non-dextran carbohydrates, a number of which can be components of the ECM. The ECM can further be contacted by DNase to remove cellular nucleic acids. The manufacture of the ECM in some embodiments can thus be without animal or plant proteins using xeno-free culture medium, and without Pinney's use for modulating cell expansion ex vivo.

### Manufacturing ECM Using Fibroblasts Generated from Pluripotent Cells

In some embodiments, a method of manufacturing extracellular matrix is provided. The method can comprise differentiating a pluripotent cell (such as an induced Pluripotent Stem Cell (iPSC)) into a production fibroblast. The method can comprise culturing the production fibroblast, so that the production fibroblast produces ECM. The method can comprise isolating the ECM from the production fibroblast, thus manufacturing the ECM. In some embodiments, the method comprises de-differentiating a precursor fibroblast to form the pluripotent cell prior to differentiating the pluripotent cell into the production fibroblast. In some embodiments, the pluripotent cell is expanded before it is differentiated into fibroblasts. It is noted that by expanding the pluripotent cell first, a large population of pluripotent cells can be generated from a small number of initial pluripotent cells before re-differentiating the pluripotent cells into fibroblasts. In some embodiments, the pluripotent cell is expanded for at least about 10 doublings, for example about or at least about 10, 15, 20, 25, or 30 doublings, included ranges between any two of the listed values, for example about 10-30 doubles, about 10-25 doublings, about 10-20 doublings, about 15-30 doublings, about 15-25 doublings, about 15-20 doublings, or about 20-30 doublings. In some embodiments, the pluripotent cell is expanded for at least about 15 doublings. It is noted that the ability of pluripotent cells to undergo numerous doublings can offer an advantage over conventional sources of fibroblasts such as neonatal foreskin cells, which typically only undergo about 10-12 doublings before they exhaust and senesce.

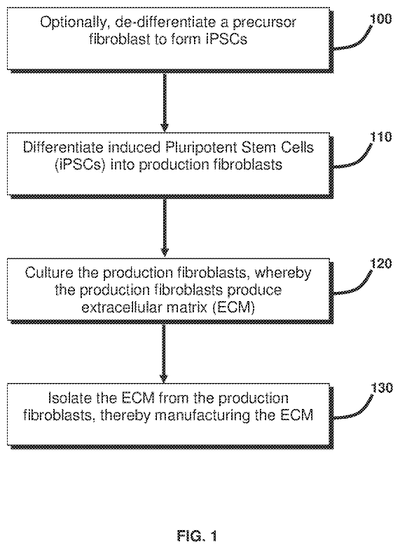

FIG. 1 is a flow diagram illustrating methods of manufacturing ECM using fibroblasts differentiated from iPSCs in accordance with some embodiments herein. In the method, optionally, a precursor fibroblast can be de-differentiated to form iPSCs 100. In the method, induced Pluripotent Stem Cells (iPSCs) can be differentiated into production fibroblasts. 110. In the method, the production fibroblasts can be cultured, whereby the production fibroblasts produce extracellular matrix (ECM). 120. In the method, the production fibroblasts can be cultured, whereby the production fibroblasts produce extracellular matrix (ECM). 130.

In methods, kits, and compositions of some embodiments, precursor fibroblasts (or iPSCs) from a single donor are used to make the ECM. Advantageously, manufacturing ECM from a single donor can minimize the number of potential contaminants (such as viral contaminants). As such, in some embodiments, the only iPSCs that are differentiated into production fibroblasts are from a single donor. It is also noted that virus-free (for example retrovirus-fee) pluripotent cells can further minimize risk of contamination or immunogenicity. Accordingly, in some embodiments, the iPSCs are free of viral insertions encoding de-differentiation factors such as an Oct family member, a Sox family member, and a Klf family member as described herein. In some embodiments, the method does not comprise using any of: an embryonic stem (ES) cell, a bone marrow Pluripotent stem cell (MSC), an ES-derived MSC, or a non-multipotent neonatal foreskin fibroblast cell line. In some embodiments, the method does not comprise using any of: an embryonic stem (ES) cell, a bone marrow Pluripotent stem cell (MSC), an ES-derived MSC, or any neonatal foreskin fibroblast cell line. In some embodiments, iPSCs are footprint free, for example iPSCs that were de-differentiated using chemical de-differentiation factors or mRNA de-differentiation factors.

It is noted that the pluripotent cell can be differentiated into a production fibroblast using differentiation factors as described herein. In some embodiments, the pluripotent cells are expanded prior to differentiating than into production fibroblasts. In some embodiments, expanding the pluripotent cells prior to differentiation can provide a large quantity of cells from a single donor, which can be differentiated to yield a large quantity of production fibroblasts from a single donor. In some embodiments, following the expansion, but prior to differentiation into production fibroblasts, the pluripotent cells are banked. For example, the pluripotent cells can be banked by freezing in liquid nitrogen.

In some embodiments, the production fibroblasts are cultured in a medium comprising, consisting essentially of, or consisting of terminally-differentiated cells (for example, the production fibroblasts themselves). In some embodiments, the production fibroblasts are cultured in a medium that is substantially free or free of mesenchymal stem cells (MSCs). Without being limited by theory, it is contemplated that fibroblasts differentiated from fibroblast-derived iPSCs in accordance with methods, compositions, and kits of some embodiments herein can be effective for efficient, large-scale production of mature ECM. For example, de-differentiating, expanding, and re-differentiating single donor iPSC's into fibroblasts in accordance with some embodiments can yield commercial scales of ECM-producing fibroblast with minimum risk of contamination or disease transmission.

In some embodiments, the method is performed without any of: an embryonic stem (ES) cell, a bone marrow multipotent stem cell (MSC), an ES-derived MSC, a non-multipotent neonatal foreskin fibroblast cell line, or any neonatal foreskin fibroblast line, or two or more of these. In some embodiments, the method is performed without any of: an embryonic stem (ES) cell, a bone marrow multipotent stem cell (MSC), an ES-derived MSC, or a non-multipotent neonatal foreskin fibroblast cell line, or two or more of these. In some embodiments, the method is performed without any of these. In some embodiments, the method is performed at a commercial scale, thus achieving commercial-scale production of ECM. As noted above, commercial scaling of primary cells, for example primary bone marrow MSC's, or neonatal foreskin-derived cells can require cells from multiple donors, raising a risk of contamination or disease transmission. Also as noted above, ES cells can raise risks of disease transmission, as well as be subject to restricted availability and use, for example due to ethical considerations. Furthermore, MSCs such as bone marrow MSCs can remain multipotent, and thus be subject to further differentiation. On the other hand, methods, compositions, and kits in some embodiments can be performed with differentiated fibroblasts (but not MSCs), so that, advantageously, the fibroblasts are not subject to further differentiation.

In some embodiments, the method comprises de-differentiating a precursor fibroblast to form the iPSC. The precursor fibroblast can be contacted with de-differentiation factors, thereby de-differentiating the precursor fibroblast into an iPSC. In some embodiments, the precursor fibroblast comprises an adult dermal (biopsy) fibroblast. In some embodiments, the precursor fibroblast is from a single donor. In some embodiments, the iPSCs are free of viral insertions encoding an Oct family member, a Sox family member, or a Klf family member. In some embodiments, the iPSCs are footprint-free. The iPSCs can then be expanded and differentiated into the production fibroblasts.

The culturing of the production fibroblasts can be performed in the presence of oxygen. Without being limited by theory, it is contemplated that culturing in normoxia is advantageous because oxygen facilitates efficient and maximal conversion of carbon sources, which are involved in all or essentially all metabolic processes. At commercial scales, oxygen is a reactant in the reaction that converts glucose and glutamine into ATP, which supports synthesis of collagens, among other proteins. In low oxygen, cells are forced to rely much more on glycolysis, rather than aerobic respiration, leading to less efficient synthesis of proteins such as collagens. As such, in some embodiments, the culturing of the production fibroblasts is performed in normoxia. In some embodiments, the culturing of the production fibroblasts is not performed in hypoxia. Hypoxia generally refers to a lower oxygen concentration as compared to the oxygen concentration of ambient air (normoxia; approximately 15%-20% oxygen). In some embodiments, hypoxic conditions include an oxygen concentration less than about 10%. In some embodiments, hypoxic conditions are characterized by an oxygen concentration of about 1% to 10%, 1% to 9%, 1% to 8%, 1% to 7%, 1% to 6%, 1% to 5%, 1% to 4%, 1% to 3%, or 1% to 2%. Hypoxic conditions can be created and maintained by using a culture apparatus that allows one to control ambient gas concentrations, for example, an anaerobic chamber.

In some embodiments, the production fibroblasts are cultured in the presence of a substrate. In some embodiments, the substrate comprises a polymer, for example a plastic surface or dextran. In some embodiments, the substrate comprises a dextran microcarrier.

In some embodiments, for example in which the production fibroblasts are cultured in the presence of dextran microcarriers, isolating the ECM from the production fibroblast comprises washing the ECM in an acidic buffer, and contacting a solution comprising the production fibroblast and ECM with dextranase. The solution comprising the production fibroblast and ECM can also be contacted by a DNase. By way of example, the dextranase can comprise a bacterial dextranase. In some embodiments, the dextranase is provided at a concentration of between 1 to 1000 U/ml. It is contemplated that the dextranase and DNase can thus facilitate the purification of the ECM by removing other substances. In some embodiments, purifying the human ECM comprises washing with an acidic buffer at a pH of 6.0 to 6.5 to remove residual culture medium components. The acid-washed ECM can be contacted with a solution comprising between 1 and 1000 U/ml bacterial dextranase and between 1 and 1000 U/ml recombinant human DNse in an acidic solution at pH between 2.0 and 7.0, or preferably between pH 6.0 to 6.5, wherein enzymatic activity is sufficient for removing the dextran beads and cellular nucleic acids. Optionally, the purification can be performed after in-process testing of the ECM.

In some embodiments, upon isolation from production fibroblasts, the ECM is no longer in fluid communication with the production fibroblast. For example, the ECM can be in a separate container from the production fibroblast. In some embodiments, isolating the ECM from the production fibroblast comprises purifying the ECM, so as to manufacture a composition that is at least 20% (w/w) ECM, for example about or at least about 20%, 25%, 30%, 35%, 40%, 45%, 50%, 55%, 60%, 65%, 70%, 75%, 80%, 85%, 90%, 95%, 97%, or 99% ECM, including ranges between any two of the listed values. In some embodiments, the composition comprises at least about 80% ECM. In some embodiments, the composition comprises at least about 80%-95% ECM.

The ECM can comprise mature ECM components as described herein. “Mature ECM” is used herein in accordance with its ordinary meaning in the field, and include cross-linked ECM, ECM that comprises a c-terminal propeptide of COL1, a triple-helical or non-reducible gamma-form fibrillary collagen, or a combination of two or more of these features. In some embodiments, the mature ECM comprises collagen. In some embodiments, about 90% (w/w) of the ECM comprises COL1, and about 10% is selected from the group consisting of: COL3, COL4, COL5, COL6, or a combination of any of these or all of these. Example sequences of Homo sapiens COL1, COL3, COL4, COL5, and COL6 include, but are not limited to the sequences shown in Table 1 herein. In some embodiments, at least about 80% of the mature ECM comprises COL1, and at least about 10% is selected from the group consisting of: COL3, COL4, COL5, COL6, and a combination of any of these. In some embodiments, at least about 85% of the mature ECM comprises COL1 (for example, at least about 85%, 87%, 90%, or 95%), and at least about 5% (for example, at least about 5%, 10%, 13%, or 15%) is selected from the group consisting of: COL3, COL4, COL5, COL6, and a combination of any of these. In some embodiments, the mature ECM comprises a c-terminal propeptide of COL1, or a triple-helical or non-reducible gamma-form fibrillar collagen, or both. It is noted that different molecules of mature ECM, for example collagen molecules as described herein, can be readily detected using an ELISA, among other assays. In some embodiments, an antibody specific for a collagen protein (or C-terminal propeptide of COL1) is used in a quantitative ELISA to ascertain amounts of components of mature ECM.

In some embodiments, the method further comprises contacting the production fibroblasts with serum until the production fibroblasts produce mature collagens. Without being limited by theory, it is contemplated that contacting the production fibroblasts with serum can support ECM deposition, increase bioproduction, and induce the production of mature ECM, for example cross-linked ECM. Then the amount of serum can be gradually reduced until there is a at least 95% reduction in the concentration of serum. In some embodiments, the gradual reducing is over a period of at least about 5 days, for example about or at least about 5, 7, 10, 12, 15, 20, 25, 30, 35, 40, 45, or 50 days, including ranges between any two of the listed values.

In some embodiments, to initiate a production lot of fibroblasts, a vial of the pluripotent cells (e.g., iPSCs generated by contacting fibroblasts with de-differentiation factors) is expanded to suitable numbers, and pluripotent cells are then induced to de-differentiate into fibrillar collagenous ECM-producing cells such as MSCs or fibroblasts, and these cells are utilized to produce the mature ECM. As such, in some embodiments, a method for manufacturing ECM comprises (a) differentiating fibroblast skin biopsy or blood samples, (b) subsequently inducing pluripotency via a chemical, polypeptide, or nucleic acid-based vector-free/footprint-free induced pluripotency, (c) isolating clones and expand the cells in the pluripotent state, (d) generating cell banks (optionally including characterization of the cells and adventitious agent testing), (e) expanding pluripotent cells for initiating a production lot for ECM, and/or (f) de-differentiating the pluripotent cells back to a differentiated state that produces sufficient mature fibrillar collagenous ECM which is insoluble in culture.

In some embodiments, a kit for manufacturing ECM is provided. The kit can comprise a composition comprising human fibroblasts, de-differentiation factors; and fibroblast differentiation factors. In some embodiments, the composition comprising human fibroblasts comprises frozen human fibroblasts. In some embodiments, all of the fibroblasts of the composition are from a single donor. In some embodiments, the kit further comprises a substrate, for example dextran microcarriers. In some embodiments, the kit further comprises dextranase and DNAase, which can be useful, for example, in isolating ECM from cells and cell culture. In some embodiments, the kit comprises pluripotent cells such as iPSC's instead of the human fibroblasts and de-differentiation factors but still comprise differentiation factors to differentiate the pluripotent cells into fibroblasts).

Some embodiments include a composition comprising at least about 80% (w/w) extracellular matrix, in which the extracellular matrix is manufactured according to any one of the methods described above. In some embodiments, the composition comprises about or at least about 60% (w/w) ECM, for example at least about 60%, 65%, 70%, 75%, 80%, 85%, 90%, 95%, 97%, or 99% ECM's, including ranges between any two of the listed values. In some embodiments, the ECM of the composition comprises, consists essentially of, or consists of mature ECM as described herein.

In some embodiments, a cell culture comprises a plurality of iPSC-derived fibroblasts in which the iPSC-derived fibroblasts are producing extracellular matrix. The cell culture can further comprise de-differentiation factors as described herein. In some embodiments, at least about 5% of the composition (w/w) is ECM, for example at least about 5%, 10%, 15%, 20%, 25%, 30%, 35%, 40%, 45%, 50%, 55%, 60%, 65%, 70%, or 75%, including ranges between any two of the listed values. In some embodiments, about 90% (w/w) of the ECM comprises COL1, and at least about 10% is selected from the group consisting of: COL3, COL4, COL5, COL6, and a combination of any of these. In some embodiments, at least about 80% of the ECM comprises COL1, and at least about 10% is selected from the group consisting of: COL3, COL4, COL5, COL6, and a combination of any of these. In some embodiments, at least about 85% of the ECM comprises COL1 (for example, at least about 85%, 87%, 90%, or 95%), and at least about 5% (for example, at least about 5%, 10%, 13%, or 15%) is selected from the group consisting of: COL3, COL4, COL5, COL6, and a combination of any of these. In some embodiments, the ECM comprises a c-terminal propeptide of COL1, or a triple-helical or non-reducible gamma-farm fibrillar collagen, or both.

### Production of Mature, Xeno-Free ECM

Some embodiments include a method of manufacturing extracellular matrix (ECM). The method can comprise culturing fibroblasts and/or mesenchymal stem cells (MSCs) on a substrate. The substrate can comprise at least two surfaces. The culturing can be performed serum-free and xeno-free, until the fibroblasts and/or MSCs define a three-dimensional shape over the at least two-surfaces and at least 80% of fibroblasts and/or MSCs arrest their cell cycle (for example, at least 80%, 85%, 90%, 93%, 95%, 97%, or 99%). The fibroblasts and/or MSCs can then be contacted with serum for about or at least about two weeks (for example, at least about two, three, four, five, six, seven, or eight weeks). As a result of contact with the serum, the fibroblasts and/or MSCs can produce soluble mature ECM, thus producing a solution comprising soluble mature ECM and the fibroblasts and/or MSCs. The solution can be xeno-free. The method can further include isolating the soluble mature ECM from the fibroblasts and/or MSCs, thus manufacturing the ECM, in which the ECM is mature xeno-free ECM. For example, isolating the soluble mature ECM can comprise collecting, a soluble fraction of the solution, which comprises spent medium. It is also contemplated that in some embodiments, the method can be performed so that the fibroblasts and/or MSCs are in media that contains serum at the start of the culturing (rather than adding serum later on). It is noted that while fibroblasts and ECMs are mentioned above, it is expressly contemplated the method can also be performed with an ECM-producing cells described herein.

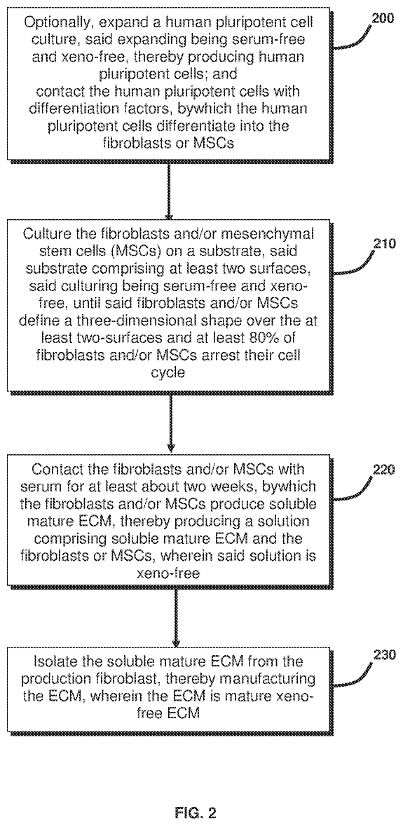

FIG. 2 is a flow diagram illustrating methods of manufacturing ECM comprising culturing fibroblasts or MSCs for at least two weeks in accordance with some embodiments herein. In the method, optionally, a human pluripotent cell culture can be expanded. Said expanding can be serum-free and xeno-free, thereby producing human pluripotent cells. An the human pluripotent cells can be contacted with differentiation factors, by which the human pluripotent cells differentiate into the fibroblasts or MSCs. 200. In the method, the fibroblasts and/or mesenchymal stem cells (MSCs) can be cultured on a substrate. The substrate can comprise at least two surfaces. The culturing can be serum-free and xeno-free, and can be performed until the fibroblasts and/or MSCs define a three-dimensional shape over the at least two-surfaces and at least 80% of fibroblasts and/or MSCs arrest their cell cycle. 210. In the method, the fibroblasts and/or MSCs can be contacted with serum for at least about two weeks, by which the fibroblasts and/or MSCs produce soluble mature ECM, thereby producing a solution comprising soluble mature ECM and the fibroblasts or MSCs, wherein said solution is xeno-free. 220. In the method, the soluble mature ECM can be isolated from the production fibroblast, thereby manufacturing the ECM, wherein the ECM is mature xeno-free ECM. 230.

In some embodiments, the method further comprises expanding a human pluripotent cell culture (e.g., iPSCs, such as footprint-free iPSCs as described herein). The expanding can be performed in serum-free and xeno-free conditions, and can produce an expanded quantity of human pluripotent cells. The method can further comprise contacting the expanded human pluripotent cells with differentiation factors as described herein. The differentiation factors can be suitable for differentiating the human pluripotent cells into the fibroblasts or MSCs.

It has been observed herein that culturing fibroblasts and/or MSCs with serum for at least 2 weeks or more unexpectedly yielded production of mature ECM comprising collagen cross-linking, and with superior solubility. Accordingly, in some embodiments, contacting the fibroblasts and/or MSCs with serum is performed for at least about 2 weeks, for example, at least about 2 weeks, 3 weeks, 4 weeks, 5 weeks, 6 weeks, 7 weeks, 8 weeks, 9 weeks, 10 weeks, 11 weeks, or 12 weeks, including ranges between any two of the listed values. In some embodiments, contacting the fibroblasts and/or MSCs with serum is performed for at least about 8 weeks. In some embodiments, contacting fibroblasts and/or MSCs with serum is performed for about 2 weeks to about 12 weeks, for example about 2 weeks, 3 weeks, 4 weeks, 5 weeks, 6 weeks, 7 weeks, 8 weeks, 9 weeks, 10 weeks, 11 weeks, or 12 weeks, including ranges between any two of the listed values, for example about 2 weeks to about 11 weeks, about 2 weeks to about 10 weeks, about 2 weeks to about 8 weeks, about 2 weeks to about 6 weeks, about 2 weeks to about 4 weeks, about 3 weeks to about 12 weeks, about 3 weeks to about 10 weeks, about 3 weeks to about 8 weeks about 3 weeks to about 6 weeks, about 4 weeks to about 12 weeks, about 4 weeks to about 10 weeks, about 4 weeks to about 8 weeks, about 4 weeks to about 6 weeks, about 6 weeks to about 12 weeks, about 6 weeks to about 10 weeks, or about 6 weeks to about 8 weeks. In some embodiments the contacting is performed for about 2 weeks to about 8 weeks. In some embodiments the contacting is performed longer than 2 weeks. In some embodiments, the fibroblasts and/or MSCs are cultured with serum under normoxic conditions. In some embodiments, the fibroblasts and/or MSCs are not cultured with serum under hypoxic conditions.

In some embodiments, the human pluripotent cells comprise induced pluripotent stem cells (iPSCs). In some embodiments, the iPSCs are footprint-free. In some embodiments, the iPSCs are from a single donor. As discussed herein, the use of iPSCs can allow pluripotent cells to be expanded, and optionally banked, prior to being differentiated into ECM-producing cells such as fibroblasts in accordance with methods, compositions, and kits of some embodiments. Accordingly, as the iPSCs can come from a single donor and then be expanded, such uses of iPSCs can permit commercial-scale quantities of ECM-producing cells from a single donor.

In some embodiments, the method further comprises manufacturing a cosmetic composition comprising the mature xeno-free ECM. In some embodiments, spent medium can also be useful for the manufacture of some cosmetic compositions.

When serum is used in or added to a culture of ECM-producing cells, such as fibroblasts in accordance with methods, compositions, and kits described herein, ascorbic acid can also be useful, for example for maintenance of the health of cells in culture. Without being limited by theory, is noted that ascorbic acid can be required for certain post-translational modifications of collagen that stabilize the mature triple-helical collagens. By way of example, ascorbic acid can be a cofactor for some enzymes such as prolyl hydroxylase. Accordingly, in some embodiments, the method further comprises contacting the fibroblasts or MSCs with ascorbic acid during the time that the cells are contacted with the serum (e.g., at least two weeks).

Without being limited by theory, it is contemplated that cells such as fibroblasts and/or MSCs can support ECM deposition and maturation once they reach a sufficient density on a substrate (such as a scaffold or support, for example dextran microcarriers as described herein). Furthermore, as the cells start to reach a sufficient density, their cell cycles can arrest (i.e. the cells can enter the G₀ phase of the cell cycle). Accordingly, in some embodiments, the fibroblasts or MSCs are not contacted with serum until they have reached a suitable density to support ECM deposition and maturation. In some embodiments, the fibroblasts or MSCs are not contacted with serum until these fibroblasts or MSCs are disposed on at least two-surfaces of a substrate defining a three-dimensional shape, and at least about 70% of the fibroblasts or MSCs arrest their cell cycles. In some embodiments, the fibroblasts or MSC's are not contacted with serum until these fibroblasts or MSCs are disposed on at least two-surfaces of a substrate defining a three-dimensional shape and at least about 70%, 75%, 80%, 85%, 90%, 95%, 97%, or 99% of the fibroblasts or MSCs arrest their cell cycles, including ranges between any two of the listed values.

A number of suitable forms of serum can be contacted with the fibroblasts and/or MSCs, for example tested clinical-grade bovine calf serum or pooled human serum from expired blood units, or a combination of these. In some embodiments, contacting the serum with the fibroblasts comprises adding the serum to the solution that comprises the fibroblasts and/or MSCs. “Adding,” is used broadly herein, and includes the addition of serum to solution containing the fibroblasts and/or MSCs, as well as the addition of solution containing fibroblasts and/or MSCs to serum. In some embodiments, serum is added to solution containing the fibroblasts and/or MSCs until the amount of serum (v/v) is about 0.1% to 10%, for example about 1-2%, 1-5%, 2-10%, 2-5%, 3-10%, 3-5%, or 5-10%.

Advantageously, producing xeno-free ECM in accordance with some embodiments herein can reduce the risk of adverse immune reactions in users of products that contain the ECM. Such products can further enjoy advantages such as streamlined regulatory review. In some embodiments, the fibroblasts and/or MSC's are derived from human pluripotent cells (e.g. IBC's) of a cell line that was previously grown using animal components. In some embodiments, the pluripotent cells are from a single donor. The pluripotent cells can be expanded without the use of xenogenic components. Thus, a large quantity of xeno-free cells can be obtained. Xeno-free pluripotent cells, in accordance with some embodiments, can then be differentiated into ECM-producing cells, for example fibroblasts and/or MSC's. The differentiation can comprise contacting the xeno-free pluripotent cells with differentiation factors as described herein.

Moreover, the xeno-free ECM manufactured in accordance with methods, compositions, and kits of some embodiments can comprise mature ECM, as described herein. As such, the mature xeno-free ECM can be well-suited for a number of cosmetic and medical products. In some embodiments, the mature xeno-free ECM comprises fibrillar collagen. In some embodiments, the mature xeno-free ECM comprises a c-terminal propetide of COL1, or a triple-helical or non-reducible gamma-form fibrillar collagen, or both.

In some embodiments, the solution comprises at least about 100 μg of collagen per cm² of the substrate, for example at least about 100 μg of collagen per cm², 150 μg of collagen per cm², 200 μg of collagen per cm², 250 μg of collagen per cm², 300 μg of collagen per cm², 350 μg of collagen per cm², 400 of collagen per cm², 450 μg of collagen per cm², 500 μg of collagen per cm², 600 μg of collagen per cm², 700 μg of collagen per cm², 800 μg of collagen per cm², 900 of collagen per cm², or 1000 μg of collagen per cm², including ranges between any two of the listed values. In some embodiments, the solution comprises at least 250 μg of collagen per cm² of the substrate.

In some embodiments, the manufactured mature xeno-free ECM comprises at least about 100 μg of collagen per cm² of the substrate, for example at least about 100 μg of collagen per cm², 150 μg of collagen per cm², 200 μg of collagen per cm², 250 μg of collagen per cm², 300 μg of collagen per cm², 350 μg of collagen per cm², 400 μg of collagen per cm², 450 μg of collagen per cm², 500 μg of collagen per cm², 600 μg of collagen per cm², 700 μg of collagen per cm², 800 μg of collagen per cm², 900 μg of collagen per cm², or 1000 μg of collagen per cm², including ranges between any two of the listed values. In some embodiments, the manufactured mature xeno-free ECM comprises comprises at least 250 μg of collagen per cm² of the substrate.

In some embodiments, the method comprises detecting an amount of mature ECM in the solution. In some embodiments, the detecting is performed using an ELISA to detect the presence, absence, and or levels of one or more components of mature ECM as described herein.

Some embodiments include a solution comprising fibroblasts or MSCs and the soluble mature ECM produced according to any one the above methods. The solution can be xeno-free, and the soluble mature ECM can comprises cross-linked collagen. In some embodiments, the solution comprises fibroblasts, but not MSCs. In some embodiments, the fibroblasts or MSCs are over a substrate in the solution, and the solution comprises at least about 100 μg of collagen per cm² of the substrate, for example at least about 100 μg of collagen per cm², 150 μg of collagen per cm², 200 μg of collagen per cm², 250 μg of collagen per cm², 300 μg of collagen per cm², 350 μg of collagen per cm², 400 μg of collagen per cm², 450 μg of collagen per cm², 500 μg of collagen per cm², 600 μg of collagen per cm², 700 μg of collagen per cm², 800 μg of collagen per cm², 900 μg of collagen per cm², or 1000 μg of collagen per cm², including ranges between any two of the listed values. In some embodiments, the solution comprises at least 250 μg of collagen per cm² of the substrate.

Producing ECM for periods of time as described in accordance with some embodiments herein, can produce higher abundances of mature ECM than conventional methods. For example, culturing the cells for about 8-12 weeks before collecting spent medium with soluble ECM, in accordance with some embodiments herein, can produce more ECM than conventional methods. On the other hand, production of embryonic-like ECM under hypoxic conditions according to some conventional methods can yield less ECM overall, and without necessarily producing any mature ECM. The amounts of ECM produced by a particular method can be measured, for example, by SDS-PAGE gel comparing adult-tissue derived type I collagen to ECM produced by the subject cell cultures).

### Manufacture of ECM Comprising Gradual Removal of Serum

Some embodiments include a method of manufacturing extracellular matrix (ECM). The method can include providing fibroblasts in a medium that comprises a non-zero concentration of serum. The method can include gradually reducing the amount of serum in the medium comprising fibroblasts until the medium contains no more than 5% of the concentration of serum, for example, no more than 5%, 4%, 3%, 2%, or 1%. This gradual reduction in serum may also be referred to herein as “serum weaning.” After the gradual reduction in serum, the method can include culturing the fibroblasts for at least about 1 week (for example at least about 1, 2, 3, 4, 5, 6, 7, 8, 9, 10, 11, or 12, weeks, including ranges between any two of the listed values), by which the fibroblasts produce soluble ECM. Thus, the method can produce a solution comprising the fibroblasts and soluble ECM. The method can further include isolating the soluble ECM from the fibroblasts, thus manufacturing the ECM. In some embodiments, the method comprises little or no cell expansion once the serum weaning has begun. In some embodiments, a quantity of fibroblasts in the medium at the start of gradually reducing the amount of serum is at least about 0.7× of a quantity of fibroblasts in the medium when the medium contains no more than 5% of the concentration in serum. In some embodiments, a quantity of fibroblasts in the medium at the start of gradually reducing the amount of serum is at least about 0.6×, 0.7×, 0.8×, 0.9×, 1×, 1.1×, or 1.2× (including ranges between any two of the listed values) of a quantity of fibroblasts in the medium when the medium contains no more than 5% of the concentration in serum. In some embodiments, a the quantity of fibroblasts in the medium at the start of gradually reducing the amount of serum is at least 0.9× of the quantity of fibroblasts in the medium when the medium contains no more than 5% of the concentration in serum. In some embodiments, a the quantity of fibroblasts in the medium at the start of gradually reducing the amount of serum is about 0.8×-1.2× of the quantity of fibroblasts in the medium when the medium contains no more than 5% of the concentration in serum. In some embodiments, gradually reducing the amount of serum (i.e., the serum weaning) is done without cell expansion or cell subculture. In some embodiments, gradually reducing the amount of serum (i.e., the serum weaning) is done without cell subculture. In some embodiments, the serum is completely removed by the gradual reduction of serum (except for trace amounts that have no appreciable effect on the culture or ECM). It is noted that while fibroblasts are mentioned above, it is expressly contemplated the method can also be performed with an ECM-producing cells described herein.

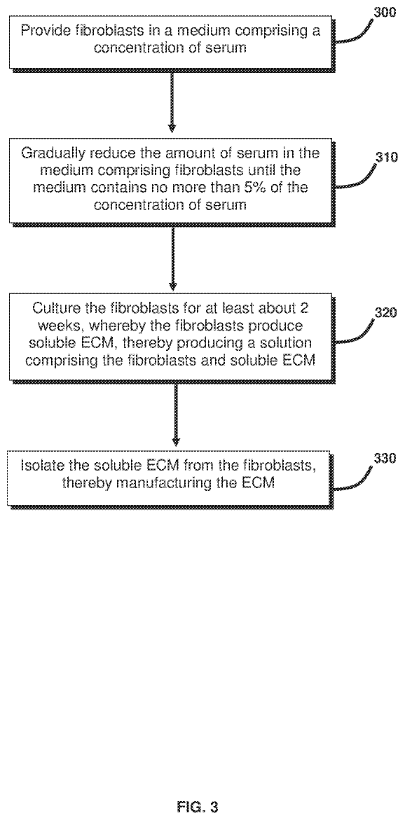

FIG. 3 is a flow diagram illustrating methods of manufacturing ECM comprising serum weaning in accordance with some embodiments herein. The ECM can be xeno-free. In the method, fibroblasts in a medium comprising a concentration of serum can be provided. 300. In the method, the amount of serum in the medium can comprising fibroblasts can be gradually reduced until the medium contains no more than 5% of the concentration of serum. 310. In the method, the fibroblasts can be cultured for at least about 2 weeks (e.g., at least about 2, 3, 4, 5, 6, 7, or 8 weeks), whereby the fibroblasts produce soluble ECM, thereby producing a solution comprising the fibroblasts and soluble ECM. 320. In the method, the soluble ECM can be isolated from the fibroblasts, thereby manufacturing the ECM. 330.

As used herein, gradually reducing the amount of serum in the medium can take place over a period of days, for example at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, including ranges between any two of the listed valued, for example about 1-30 days, 1-20 days, 1-14 days, 1-10 days, 1-7 days, 1-5 days, 1-30 days, 2-20 days, 2-14 days, 2-10 days, 2-7 days, 2-5 days, 3-20 days, 3-14 days, 3-10 days, 3-7 days, 3-5 days, 5-20 days, 5-14 days, 5-10 days, 5-7 days, 7-20 days, 7-14 days, 7-10 days, 10-20 days, or 10-14 days. In some embodiments, the serum is gradually reduced for at least about 5 days. The gradual reduction of the serum can involve removal of serum-containing medium, and replacing in with serum-free medium. By performing multiple rounds of replacing a portion of serum-containing medium with serum-free medium, the serum can be gradually reduced, until it is effectively eliminated (i.e., so that no more than trace amounts of serum remain, which have no appreciable effect on the cell culture or ECM).

Surprisingly, gradually removing the serum, until the serum is completely, (or nearly completely) removed as described in accordance with some embodiments herein can induce a majority of the ECM-producing cells to arrest the cell cycle (i.e., enter a G₀ phase), and produce soluble mature ECM without substantially degrading the ECM or inducing apoptosis. Accordingly, in some embodiments, following the gradual reducing of the serum, soluble, intact, and mature ECM is produced.

In some embodiments, following the gradual reducing of the serum, at least about 70% of the fibroblasts in the solution are in a G₀ cell cycle phase, for example at least about 70%, 75%, 80%, 85%, 90%, 95%, 97%, or 99%, including ranges between any two of the listed values, for example 70-99%, 70-95%, 70-90%, 80-99%, 80-95%, 80-90%, 85-99%, 85-95%, or 85-90%. In some embodiments, at least about 90% of the fibroblasts in the solution are in a G₀ cell cycle phase.

In some embodiments, following the gradual reducing of the serum, than about 5% of the fibroblasts in the solution are undergoing apoptosis. As used herein, a percentage of cells “undergoing” apoptosis refers to cells that exhibit detectable markers indicative of apoptosis, and thus may include cells that are in the process of apoptosis, as well as cells that have recently completed a program of apoptosis. A percentage of cells undergoing apoptosis can be measured, for example by detection of caspase cleavage, or TUNEL staining. In some embodiments, fewer than 5%, for example fewer than about 4%, 3%, 2%, 1%, 0.5%, or 0.1% of the fibroblasts in the solution are undergoing apoptosis, including ranges between any two of the listed values, for example, 0.1% to 5%, 0.1% to 3%, 0.1% to 1%, 0.5% to 5%, 0.5% to 3%, 0.5% to 1%, 1% to 5%, 1% to 3%, or 3% to 5%. In some embodiments, fewer than about 1% of the fibroblasts in the solution are undergoing apoptosis.

It, is noted that methods, compositions, and kits in accordance with some embodiments herein can produce large quantities of mature ECM. In some embodiments, large structures comprising ECM are manufactured, to the extent that some of the structures are visible under a microscope. In some embodiments, the solution comprises nanostructures comprising the soluble ECM, in which the nanostructures have a greatest diameter of at least 200 nm, and up to 10,000 nm, for example at least 200 nm, 300 nm, 400 nm 500 nm, 1000 nm, 2000 nm, 5000 nm, or more. In some embodiments, the nanostructures have a greatest diameter of 200 nm to 10,000 nm. It is noted that in view of these large diameters, filtration thorough certain filters may not be feasible, as the structures comprising ECM may readily clog the filter, causing substantial loss of recovered ECM. As such, in some embodiments, manufacturing, purifying, and/or isolating the ECM comprises purification that does not comprise filtering. In some embodiments, manufacturing the ECM comprises purification that does not comprise sterile filtering. In some embodiments, manufacturing the ECM comprises purification that does not comprise 0.1 μM filtering. In some embodiments, avoiding filtering (or sterile filtering) such as 0.1 μM filtering can substantially increase the yield of mature ECM that is recovered. In some embodiments, isolating the ECM from the fibroblasts is performed without sterile filtering solution comprising the ECM. In some embodiments, manufacturing the ECM is performed without sterile filtering solution.

Some embodiments include a solution comprising fibroblasts and soluble ECM, in which at least about 90% of the fibroblasts in the solution are in a G₀ cell cycle phase, and fewer than 1% of the fibroblasts in the solution are undergoing apoptosis. The solution can comprises nanostructures comprising the soluble ECM. The nanostructures can have a greatest diameter of 200 nm to 10,000 nm. In some embodiments, the content of serum in the solution (v/v) is less than 0.1%, for example, less than 0.1%, 0.05%, 0.01%, 0.005%, or 0.001%, including ranges between any two of the listed values, for example, a serum content of 0.1% to 0.001%. In some embodiments the solution is free of serum (as used herein, a solution that is “free” of serum may optionally contain insignificant amounts of serum that have no appreciable effects on the cell culture of production of ECM). In some embodiments the solution is manufactured according to any of the above methods.

### Additional Embodiments

Some embodiments also include systems and methods for manufacture and distribution of human ECM and products that contain ECM for cosmetic and therapeutic uses. In some embodiments, the methods and systems provide efficiencies in the manufacture and distribution of: 1) low-cost and efficient removal of majority of dextran microcarriers, sufficient for collecting and further processing of the human ECM without interference from the microcarriers, 2) desirable features for marketing and commercial implementation of xeno-free products, including methods and systems for communicating such desirable features to manufacturers and users, and 3) efficiencies in making products from both soluble and non-soluble fractions from a single manufacturing lot, which can be more resource-efficient than only using one of these fractions to manufacture a product. As such, methods and systems in accordance with some embodiments herein can provide efficiencies in the manufacture of ECM and ECM-containing products.

Additional options are set forth below:
1. A method of manufacturing extracellular matrix, the method comprising:
   differentiating induced Pluripotent Stem Cells (iPSCs) into a production fibroblast;
   culturing the production fibroblasts, whereby the production fibroblasts produce extracellular matrix (ECM); and
   isolating the ECM from the production fibroblasts, thereby manufacturing the ECM.
2. The method of option 1, further comprising de-differentiating a precursor fibroblast to form the iPSCs prior to differentiating the iPSCs into the production fibroblast.
3. The method of any one of options 1-2 further comprising expanding the iPSC's prior to said differentiating.
4. The method of any one of options 2-3, further comprising constructing a bank of the iPSC's prior to said differentiating.
5. The method of option 1, wherein the only iPSCS that are differentiated are from a single donor.
6. The method of any one of options 1-5, wherein the culturing of the production fibroblasts is in normoxia.
7. The method of any one of options 1-6, wherein culturing the production fibroblasts does not comprise culturing mesenchymal stem cells (MSCs).
8. The method of any one of options 1-7, wherein the precursor fibroblast comprises an adult dermal (biopsy) fibroblast.
9. The method of any of options 1-8, wherein isolating the ECM comprises purifying the ECM, thereby manufacturing a composition that is at least about 80% w/w ECM
10. The method of option 9, wherein purifying the ECM comprises:
   washing the ECM in an acidic buffer; and
   contacting a solution comprising the production fibroblast and ECM with dextranase.
11. The method of any one of options 1-10, wherein the ECM comprises collagen.
12. The method of option 11, wherein about 90% (w/w/) of the ECM is COL1, and about 10% is selected from the group consisting of: COL3, COL4, COL5, COL6, and a combination of any of these.
13. The method of any one of options 2-12, further contacting the precursor fibroblast with de-differentiation factors, thereby de-differentiating the precursor fibroblast into the iPSC.
14. The method of any one of options 1-13, wherein the iPSCs are free of viral insertions encoding an Oct family member, a Sox family member, a Klf family member.
15. The method of any one of options 1-13, wherein the iPSCs are footprint-free,
16. The method of any one of options 1-15, wherein the method does not comprise any of: an embryonic stem (ES) cell, a bone marrow multipotent stem cell, an ES-derived MSC, or a non-multipotent neonatal foreskin fibroblast cell line.
17. The method of any one of options 1-16, wherein the ECM comprises a c-terminal propeptide of COL1 or a triple-helical or non-reducible gamma-form fibrillar collagen, or both.
18. The method of any one of options 1-17. further comprising contacting the production fibroblasts with serum, until the production fibroblasts produce mature collagens, and then gradually reducing the amount of scrum until there is a at least 95% reduction in the concentration of serum.
19. The method of option 18, wherein the gradual reducing is over a period of at least about 5 days.
20. A kit for manufacturing ECM, the kit comprising:
   a composition comprising human fibroblasts;
   de-differentiation factors; and
   fibroblast differentiation factors.
21. The kit of option 20, wherein all of the fibroblasts of the composition are from a single donor.
22. The kit of any one of options 20-21, further comprising a substrate, such as dextran microcarriers.
23. The kit of option 22, further comprising dextranase and DNAase.
24. A composition comprising at least 80% (w/w) extracellular matrix, wherein the extracellular matrix is manufactured according to the method of any one of options 1-14.
25. A cell culture comprising:
   iPSC-derived fibroblasts, wherein the iPSC-derived fibroblasts are producing mature extracellular matrix; and
   de-differentiation factors,
   wherein at least 50% of the composition (w/w) comprises ECM.
26. A method of manufacturing extracellular matrix (ECM), the method comprising: culturing fibroblasts and/or mesenchymal stem cells (MSCs) on a substrate, said substrate comprising at least two surfaces, said culturing being serum-free and xeno-free, until said fibroblasts and/or MSCs define a three-dimensional shape over the at least two-surfaces and at least 80% of fibroblasts and/or MSCs arrest their cell cycle;
thereafter contacting the fibroblasts and/or MSCs with serum for at least about two weeks, by which the fibroblasts and/or MSCs produce soluble mature ECM, thereby producing a solution comprising soluble mature ECM and the fibroblasts or MSCs, wherein said solution is xeno-free; and
isolating the soluble mature ECM from the production fibroblast, thereby manufacturing the ECM, wherein the ECM is mature xeno--free ECM.
27. The method of option 26, further comprising:
   expanding a human pluripotent cell culture, said expanding being serum-free and xeno-free, thereby producing human pluripotent cells; and
   contacting the human pluripotent cells with differentiation factors, by which the human pluripotent cells differentiate into the fibroblasts or MSCs.
28. The method of any one of options 26-27, wherein said contacting of the fibroblasts and/or MSCs with serum is for about 2 weeks to about 8 weeks
29. The method of any one of options 26-27, wherein said contacting of the fibroblasts and/or MSCs with serum is for at least about 8 weeks
30. The method of any one of options 27-29, wherein said human pluripotent cells comprise induced pluripotent stem cells (iPSCSs).
31. The method of option 30, wherein said iPSCSs are footprint-free.
32. The method of any one of options 30-31, wherein said iPSCSs are from a single donor.
33. The method of any one of options 26-32, further comprising manufacturing a cosmetic composition comprising the mature xeno-free ECM.
34. The method of any one of options 26-33, further comprises contacting the fibroblasts or MSCs with ascorbic acid during the at least two weeks of the contacting with the serum.
35. The method of any one of options 26-34, wherein the fibroblasts or MSCs are not contacted with serum prior to said fibroblasts or MSCs over the at least two-surfaces defining a three-dimensional shape and at least 70% of the fibroblasts or MSCs arresting their cell cycle
36. The method of any one of options 26-35, wherein the amount of serum is about 0.1% to 10% (v/v)
37. The method of any one of options 26-36, wherein the amount of serum is about 1-2% (v/v).
38. The method of any one of options 26-37, wherein the serum comprises clinical-grade bovine calf serum, pooled human serum, or a combination thereof.
39. The method of any one of options 26-38, wherein human pluripotent cells are of a cell line that was previously grown using animal components.
40. The method of any one of options 26-39, wherein the mature xeno-free ECM comprises fibrillar collagen.
41. The method of option 40, wherein the mature xeno-free ECM comprises a c-terminal propeptide of COL1, or a triple-helical or non-reducible gamma-form fibrillar collagen, or both.
42. The method of any one of options 26-41, wherein the solution comprises at least 250 μg of collagen per cm² of the substrate.
43. The method of any one of option 26-42, wherein the manufactured mature xeno-free ECM comprises at least 250 μg of collagen per cm² of the substrate
44. The method of any one of options 27-43, wherein the pluripotent cells are from a single donor.
45. The method of any one of option 26-44, further comprising detecting an amount of mature ECM in the solution.
46. The method of any one of option 26-45, further comprising collecting a quantity of spent culture medium from the solution and isolating soluble mature ECM from the spent culture medium.
47. A solution comprising fibroblasts or MSCs and the soluble mature ECM produced according to any one of options 26-45, wherein said solution is xeno-free, and wherein the soluble mature ECM comprises cross-linked collagen.
48. A method of manufacturing extracellular matrix (ECM), the method comprising:
   providing fibroblasts in a medium comprising a concentration of serum;
   gradually reducing the amount of serum in the medium comprising fibroblasts until the medium contains no more than 5% of the concentration of serum;
   following said gradually reducing, culturing the fibroblasts for at least about 2 weeks, whereby the fibroblasts produce soluble ECM, thereby producing a solution comprising the fibroblasts and soluble ECM; and
   isolating the soluble ECM from the fibroblasts, thereby manufacturing the ECM.
49. The method of option 48, wherein a quantity of fibroblasts in the medium at the start of gradually reducing the amount of serum is at least 0.7× of a quantity of fibroblasts in the medium when the medium contains no more than 5% of the concentration serum.
50. The method of option 49, wherein the quantity of fibroblasts in the medium at the start of gradually reducing the amount of serum is at least 0.9× of the quantity of fibroblasts in the medium when the medium contains no more than 5% of the concentration in serum.
51. The method of any one of options 48-50, wherein gradually reducing the amount of serum is done without cell expansion or cell subculture.
52. The method of any one of options 48-51, wherein the serum is gradually reduced for at least about 5 days.
53. The method of any one of options 48-52, wherein at least about 90% of the fibroblasts in the solution are in a G₀ cell cycle phase.
54. The method of any one of options 48-53, wherein fewer than 1% of the fibroblasts in the solution are undergoing apoptosis.
55. The method of any one of options 48-54, wherein the solution comprises nanostructures comprising the soluble ECM, said nanostructures having a greatest diameter of 200 nm to 10,000 nm.
56. The method of any one of options 48-55, wherein manufacturing the ECM is performed without sterile-filtering. (so as not to exclude the nanostructures).
57. The method of any one of options 48-56 wherein isolating the soluble ECM from the fibroblasts is performed without sterile-filtering.
58. A solution comprising fibroblasts and soluble ECM, wherein at least about 90% of the fibroblasts in the solution are in a G₀ cell cycle phase, wherein fewer than 1% of the fibroblasts in the solution are undergoing apoptosis, and wherein the solution comprises nanostructures comprising the soluble ECM, said nanostructures having a greatest diameter of 200 nm to 10,000 nm.
59. The solution of option 58, wherein the soluble ECM is manufactured according to a method of any one of options 48-57.

### EXAMPLES

### Example 1

Human fibroblasts are derived from a skin biopsy of a single donor. The fibroblasts are contacted with Oct4, Sox2, and c-Myc mRNA, so as to induced them into footprint-free iPSC's. The footprint-free iPSC's are expanded from 30 doublings, and the iPSC's are banked. A commercial-scale quantity of the iPSC's are contacted with connective tissue growth factor (CTGF) so as to induce them to differentiate into fibroblasts. The fibroblasts are cultured in medium comprising dextran microcarrier substrates, and proceed to produce mature ECM. The ECM is insoluble in culture. The dextran microcarriers are digested using dextranase. A remaining insoluble fraction, comprising mature ECM, is recovered. As such, an insoluble fraction of the culture comprising the mature ECM is isolated from the fibroblasts and soluble components. Thus, mature ECM is manufactured, and is suitable for use in medical and cosmetic products.

### Example 2

Human fibroblasts are chemically de-differentiated into footprint-free iPSC's using the small molecule cocktail “VC6TF” (V, VPA; C, CHIR99021 or CHIR; 6, 616452; T, tranylcypromine; F, forskolin). The iPSC's are expanded to a commercial production scale, and banked in a liquid-nitrogen deep-freeze. A commercial quantity of iPSC's is recovered, and differentiated into fibroblasts using CTGF. The fibroblasts are cultured in serum-free, xeno-free media comprising dextran microcarrier substrates. When the fibroblasts reach a sufficient density over two or more surfaces of the substrates, about 90% the fibroblasts enter the G₀ phase of the cell cycle. At this, point pooled human serum from expired blood is added to the fibroblast culture up to a concentration (v/v) of 2% serum. Ascorbic acid is added to the culture along with the serum. The cells are cultured for an additional 8 weeks in serum, and produce mature ECM. The mature ECM is isolated from the cell culture, and subsequently used to manufacture cosmetic products.

### Example 3

Human fibroblasts are derived from single donor human iPSC's as described in Example 1. The fibroblasts are cultured in 2% fetal bovine serum, and produce mature ECM. Every day for 10 days, one-half of the serum-containing medium is replaced with serum-free medium. As such, it is estimated that after 10 days of replacement, the 2% serum has been reduced by a factor of 2¹⁰, so that the culture contains less than 0.002% serum. After the gradual serum replacement is completed, the fibroblasts are cultured in the medium (containing an estimate of less than 0.002% serum) for six weeks. Soluble ECM is recovered from spent media. Additionally, large structures (200 nm to 10,000 nm in diameter) comprising ECM are present in the media, so the isolation of ECM is performed without sterile filtering.

In some embodiments, the method use, or composition comprises various steps or features that are present as single steps or features (as opposed to multiple steps or features). For example, in one embodiment, the method includes a single administration of a flow modulator, or the composition comprises or consists essentially of a flow modulator for single use. The flow modulator may be present in a single dosage unit effective for increasing flow (or decreasing immune cell migration). A composition or use may comprise a single dosage unit of a flow modulator effective for increasing flow (or inhibiting migration of immune cells) as described herein. Multiple features or components are provided in alternate embodiments. In some embodiments, the method, composition, or use comprises one or more means for flow modulation. In some embodiments, the means comprises a flow modulator.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims. For each method of described herein, relevant compositions for use in the method are expressly contemplated, uses of compositions in the method, and, as applicable, methods of making a medicament for use in the method are also expressly contemplated. For example, for methods of increasing flow that comprise a flow modulator, flow modulators for use in the corresponding method are also contemplated, as are uses of a flow modulator in increasing flow according to the method, as are methods of making a medicament comprising the flow modulator for use in increasing flow.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. For example, “about 5”, shall include the number 5. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A method of manufacturing extracellular matrix, the method comprising:
differentiating induced Pluripotent Stem Cells (iPSCs) into a production fibroblast;
culturing the production fibroblasts, whereby the production fibroblasts produce extracellular matrix (ECM); and
isolating the ECM from the production fibroblasts, thereby manufacturing the ECM.

2. The method of claim 1, further comprising de-differentiating a precursor fibroblast to form the iPSCs prior to differentiating the iPSCs into the production fibroblast.

3. The method of claim 1, further comprising expanding the iPSC's prior to said differentiating.

4. The method of claim 1, further comprising constructing a bank of the iPSC's prior to said differentiating.

5. The method of claim 1, wherein the only iPSCs that are differentiated are from a single donor.

6. The method of claim 1, wherein the culturing of the production fibroblasts is in normoxra.

7. The method of claim 1, wherein culturing the production fibroblasts does not comprise culturing mesenchymal stem cells (MSCs).

8. The method of claim 1, wherein the iPSCs are footprint-free.

9. The method of claim 1, wherein the method does not comprise any of: an embryonic stem (ES) cell, a bone marrow multipotent stem cell, an ES-derived MSC, or a non-multipotent neonatal foreskin fibroblast cell line.

10. The method of claim 1, wherein the ECM comprises a c-terminal propeptide of COL1, or a triple-helical or non-reducible gamma-form fibrillar collagen, or both.

11. A method of manufacturing extracellular matrix (ECM), the method comprising:
culturing fibroblasts and/or mesenchymal stem cells (MSCs) on a substrate, said substrate comprising at least two surfaces, said culturing being serum-free and xeno-free, until said fibroblasts and/or MSCs define a three-dimensional shape over the at least two surfaces and at least 80% of fibroblasts and/or MSCs arrest their cell cycle;
thereafter contacting the fibroblasts and/or MSCs with serum for at least about two weeks, by which the fibroblasts and/or MSCs produce soluble mature ECM, thereby producing a solution comprising soluble mature ECM and the fibroblasts or MSCs, wherein said solution is xeno-free; and
isolating the soluble mature ECM from the production fibroblast, thereby manufacturing the ECM, wherein the ECM is mature xeno-free ECM.

12. The method of claim 11, further comprising:
expanding a human pluripotent cell culture, said expanding being serum-free and xeno-free, thereby producing human pluripotent cells; and
contacting the human pluripotent cells with differentiation factors, by which the human pluripotent cells differentiate into the fibroblasts or MSCs.

13. The method of claim 11, wherein said contacting of the fibroblasts and/or MSCs with serum is for at least about 8 weeks

14. The method of claim 11, wherein said human pluripotent cells comprise induced pluripotent stem cells (iPSCSs).

15. The method of claim 11, further comprising manufacturing a cosmetic composition comprising the mature xeno-free ECM.

16. The method of claim 11, wherein the mature xeno-free ECM comprises a cterminal propeptide of COL1, or a triple-helical or non-reducible gamma-form fibrillar collagen, or both.

17. The method of claim 11, wherein the pluripotent cells are from a single donor.

18. A method of manufacturing extracellular matrix (ECM), the method comprising:
providing fibroblasts in a medium comprising a concentration of serum;
gradually reducing the amount of serum in the medium comprising fibroblasts until the medium contains no more than 5% of the concentration of serum;
following said gradually reducing, culturing the fibroblasts for at least about 2 weeks, whereby the fibroblasts produce soluble ECM, thereby producing a solution comprising the fibroblasts and soluble ECM; and
isolating the soluble ECM from the fibroblasts, thereby manufacturing the ECM.

19. The method of claim 18, wherein a quantity of fibroblasts in the medium at the start of gradually reducing the amount of serum is at least 0.7× of a quantity of fibroblasts in the medium when the medium contains no more than 5% of the concentration in serum.

20. The method of claim 18, wherein gradually reducing the amount of serum is done without cell expansion or cell subculture.

21.-23. (canceled)
